(19) United States
(12) Patent Application Publication
(10) Pub. No.: US 2022/0187298 A1
(43) Pub. Date: Jun. 16, 2022

(54) A METHOD OF IDENTIFYING A FLAVIVIRUS INFECTION, AND RELATED PEPTIDES, KITS AND COMPOSITIONS

(71) Applicant: Agency for Science, Technology and Research, Singapore (SG)

(72) Inventors: Fong Poh Lisa Ng, Singapore (SG); Siti Naqiah Amrun, Singapore (SG); Wearn Xin Yee, Singapore (SG)

(21) Appl. No.: 17/594,349

(22) PCT Filed: Apr. 10, 2020
(86) PCT No.: PCT/SG2020/050221

§ 371 Date: Oct. 12, 2021

(30) Foreign Application Priority Data
Apr. 12, 2019 (SG) 10201903321Y

(51) Int. Cl. G01N 33/569

(52) U.S. Cl. CPC G01N 33/56983; G01N 2469/20; G01N 2333/185; C12N 2770/36134

(57) ABSTRACT

There is provided a method of identifying a flavivirus infection selected from Zika virus (ZIKV), Dengue virus (DENV) and combination thereof in a subject, the method comprising determining whether a sample of the subject reacts with a peptide associated with a certain relative binding capacity. Also claimed are kits, isolated peptides, immune system stimulating compositions comprising specific peptides and a method of distinguishing ZIKV infection from DENV infection.

### TECHNICAL FIELD

The present disclosure relates broadly to a method of identifying a flavivirus infection, and related peptides, kits and compositions.

### BACKGROUND

Flaviviridae are a family of positive, single-stranded, enveloped RNA viruses. The genus Flavivirus have been reported to cause widespread morbidity and mortality throughout the world. Some flaviviruses are transmitted through mosquitoes and they include yellow fever virus (YFV), dengue virus (DENV), Japanese encephalitis virus (JEV), West Nile virus (WNV), and Zika virus (ZIKV).

Dengue fever, caused by DENV, is found in tropical and sub-tropical climates worldwide. In recent decades, the global incidence of dengue has grown dramatically with about half of the world's population is now at risk. There are four DENV serotypes: DENV1, DENV2, DENV3, and DENV4. As the four serotypes are different, a person can be infected with DENV as many as four times in his or her lifetime.

ZIKV was first identified in Uganda and isolated from infected monkeys in 1947. ZIKV has re-emerged as an important flavivirus that has caused several Zika fever (ZIKF) epidemics worldwide. Zika virus (ZIKV) outbreaks in French Polynesia and Brazil in 2013 and 2015 resulted in unexpected severe neurological and congenital complications.

Current diagnosis of flaviviruses such as DENV and ZIKV, which relies heavily on molecular methods, poses several limitations because the patients display a short viremic phase with low viremia levels, and thus may escape detection, even in symptomatic patients. Serology, as an alternative diagnostic approach, has been hampered due to the cross-reactive nature of the antibodies between flaviviruses, such as between DENV and ZIKV which share high amino acid identity (55%) and structural homology. Moreover, as both viruses are transmitted by the same mosquito vectors, they are often found in overlapping geographical areas.

Some antigens may possibly distinguish between ZIKV infections and DENV infections, as well as other flavivirus infections. Although computational studies have predicted multiple differential epitopes, validation on patient samples however remains a challenge.

Thus, there is a need to provide an alternative method of identifying a flavivirus infection, and related peptides, kits and compositions.

### SUMMARY

In one aspect, there is provided a method of identifying a flavivirus infection selected from Zika virus (ZIKV), dengue virus (DENV) and combination thereof in a subject, the method comprising:

determining whether a sample of the subject reacts with a peptide P that is capable of giving a relative binding capacity BCᵣₑₗₐₜᵢᵥₑ≤0.05 or ≥0.1, wherein

$${BC}_{relative} = \frac{{R}_{P 1 z} - {R}_{P 2 z}}{{R}_{P 2 z}} - \frac{{R}_{P 1 d} - {R}_{P 2 d}}{{R}_{P 2 d}}$$

(i) wherein where the peptide P comprises a ZIKV-derived peptide,

R_{P1z}=response of a ZIKV-induced antigen binding protein with peptide P,

R_{P2z}=response of the ZIKV-induced antigen binding protein with a corresponding peptide C comprising a DENV-derived peptide having a sequence homologous to the peptide P,

R_{P1d}=response of a DENV-induced antigen binding protein with the peptide P,

R_{P2d}=response of the DENV-induced antigen binding protein with the corresponding peptide C comprising a DENV-derived peptide having a sequence homologous to the peptide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein ,$$

or

(ii) wherein where the peptide P comprises a DENV-derived peptide,

R_{P1z}=response of a ZIKV-induced antigen binding protein with a corresponding peptide C comprising a ZIKV-derived peptide having a sequence homologous to the peptide P,

R_{P2z}=response of the ZIKV-induced antigen binding protein with the peptide P,

R_{P1d}=response of a DENV-induced antigen binding protein with the corresponding peptide C comprising a ZIKV-derived peptide having a sequence homologous to the peptide P,

R_{P2d}=response of the DENV-induced antigen binding protein with the peptide P.

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein .$$

In one embodiment, the peptide P comprises an epitope on a prM protein, an E glycoprotein or a NS1 protein of ZIKV or DENV.

In one embodiment, the epitope is located in a solvent-exposed region of the prM protein, the E glycoprotein or the NS1 protein.

In one embodiment, the peptide P is from 5 to 25 amino acids long.

In one embodiment, the peptide P shares no more than 50% sequence similarity with the corresponding peptide C.

In one embodiment, the peptide P comprises one or more ZIKV-derived peptide selected from the group consisting of:

SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);

a peptide sharing at least 75% sequence identity thereto; or

a peptide differing by one, two, three or four amino acids thereto; or

portions thereof; and/or

one or more DENV-derived peptide selected from the group consisting of:

SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);

a peptide sharing at least 75% sequence identity thereto; or

a peptide differing by one, two, three or four amino acids thereto; or portions thereof.

In one embodiment, the method comprises determining whether the sample reacts with one or more peptide selected from the group consisting of:

SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);

a peptide sharing at least 75% sequence identity thereto; or

a peptide differing by one, two, three or four amino acids thereto; or

portions thereof; and/or

wherein where the sample reacts with the one or more peptide or the portions thereof, the subject is indicated for flavivirus infection.

In one embodiment, the method is a method of identifying a flavivirus infection in an acute phase, and the method comprises determining whether the sample reacts with one or more peptide selected from the group consisting of:

SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);

wherein where the sample reacts with the one or more peptide or the portions thereof, the subject is indicated for flavivirus infection in an acute phase.

In one embodiment, the method comprises determining whether the sample reacts with one or more peptide selected from the group consisting of:

SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);

a peptide sharing at least 75% sequence identity thereto; or

a peptide differing by one, two, three or four amino acids thereto; or

portions thereof;

wherein where the sample reacts with the one or more peptide or the portions thereof, the subject is indicated for ZIKV infection.

In one embodiment, the method is a method of identifying a ZIKV infection in an acute phase, and the method comprises determining whether the sample reacts with:

SEQ ID NO: 32
(FKSLFGGMSWFSQILIGT);

a peptide sharing at least 75% sequence identity thereto; or

a peptide differing by one, two, three or four amino acids thereto; or

portions thereof;

wherein where the sample reacts with the peptide or the portions thereof, the subject is indicated for ZIKV infection in an acute phase.

In one embodiment, the method comprises determining whether the sample reacts with one or more peptide selected from the group consisting of:

SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);

and/or

a peptide sharing at least 75% sequence identity thereto; or

a peptide differing by one, two, three or four amino acids thereto; or

portions thereof;

wherein where the sample reacts with the one or more peptide or the portions thereof, the subject is indicated for DENV infection.

In one embodiment, determining whether the sample reacts with the peptide P comprises performing an immunoassay to assess whether antigen-binding proteins that are capable of binding to the peptide are present in the sample.

In one embodiment, the method further comprising administering to the subject a ZIKV and/or DENV treatment regimen if the subject is indicated for ZIKV and/or DENV infection.

In one aspect, there is provided a kit for identifying a flavivirus infection selected from Zika virus, dengue virus and combination thereof in a subject, the kit comprising a peptide P that is capable of giving a relative binding capacity BCᵣₑₗₐₜᵢᵥₑ≤0.05 or ≥0.1, wherein

$${BC}_{relative} = \frac{{R}_{P 1 z} - {R}_{P 2 z}}{{R}_{P 2 z}} - \frac{{R}_{P 1 d} - {R}_{P 2 d}}{{R}_{P 2 d}}$$

wherein where the peptide P comprises a ZIKV-derived peptide,

R_{P1z}=response of a ZIKV-induced antigen binding protein with peptide P,

R_{P2z}=response of the ZIKV-induced antigen binding protein with a corresponding peptide C comprising a DENV-derived peptide having a sequence homologous to the peptide P,

R_{P1d}=response of a DENV-induced antigen binding protein with the peptide P,

R_{P2d}=response of the DENV-induced antigen binding protein with the corresponding peptide C comprising a DENV-derived peptide having a sequence homologous to the peptide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein ,$$

wherein where the peptide P comprises a DENV-derived peptide,

R_{P1z}=response of a ZIKV-induced antigen binding protein with a corresponding peptide C comprising a ZIKV-derived peptide having a sequence homologous to the peptide P,

R_{P2z}=response of the ZIKV-induced antigen binding protein with the peptide P,

R_{P1d}=response of a DENV-induced antigen binding protein with the corresponding peptide C comprising a ZIKV-derived peptide having a sequence homologous to the peptide P,

R_{P2d}=response of the DENV-induced antigen binding protein with the peptide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein .$$

In one embodiment of the kit, wherein the peptide P comprises

one or more ZIKV-derived peptide selected from the group consisting of:

SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);

a peptide sharing at least 75% sequence identity thereto; or

a peptide differing by one, two, three or four amino acids thereto; or

portions thereof; and/or

one or more DENV-derived peptide selected from the group consisting of:

SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);

or

a peptide sharing at least 75% sequence identity thereto; or

a peptide differing by one, two, three or four amino acids thereto; or

portions thereof.

In one embodiment, the kit further comprises one or more of the following:

a plate coated with a capture agent for anti-ZIKV and/or anti-DENV, and

a detection agent for detecting the presence of captured anti-ZIKV and/or anti-DENV,

wherein the capture agent and the detection agent comprise a ZIKV and/or DENV antigen and/or an anti-immunoglobulin.

In one embodiment, the subject comprises an Asian subject.

In one aspect, there is provided an isolated peptide selected from the group consisting of:

SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);

or

a peptide sharing at least 75% sequence identity thereto; or

a peptide differing by one, two, three or four amino acids thereto; or

portions thereof.

In one aspect, there is provided an immune system stimulating composition comprising a peptide selected from the group consisting of:

SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);

or

a peptide sharing at least 75% sequence identity thereto; or

a peptide differing by one, two, three or four amino acids thereto; or

portions thereof.

In one aspect, there is provided a method of distinguishing ZIKV infection from DENV infection in a subject, the method comprising:

determining whether a sample of the subject reacts with a peptide of SEQ ID NO: 32 (FKSLFGGMSWFSQILIGT); a peptide sharing at least 75% sequence identity thereto; or a peptide differing by one, two, three or four amino acids thereto; or portions thereof, wherein where the sample reacts with the peptide or the portions thereof, the subject is indicated for ZIKV infection; and/or

determining whether a sample of the subject reacts with a peptide of SEQ ID NO: 9 (YGTCHHKKGEARRSR); a peptide sharing at least 75% sequence identity thereto; or a peptide differing by one, two, three or four amino acids thereto; or portions thereof, wherein where the sample reacts with the peptide or the portions thereof, the subject is indicated for DENV infection.

### Definitions

The term “identifying” as used herein in relation to an infection is to be interpreted broadly to encompass determining a presence, an absence, an amount, or a level of disease burden of the infection. The infection may be an infection in an acute phase, an early convalescent phase, a late convalescent phase, an early recovery phase, a late recovery phase or a full recovery phase.

The term “peptide” as used herein broadly refers to any chain of amino acid residues connected via peptide bonds. The peptide may be naturally occurring or synthetic (e.g., generated by chemical synthesis or recombinant DNA technology). No particular size is implied by the term “peptide”. In some examples, peptide may not include a whole virus or a full-length antigen.

The term “derived” as used herein in relation to a peptide is intended to indicate that the amino acid sequence of the peptide originated from the source specified, but has not necessarily been obtained directly from the specified source. For example, a “DENV-derived peptide” refers to a peptide which amino acid sequence is similar or identical to a part or a fragment of a naturally occurring DENV, or similar or identical to a part or a fragment of a consensus sequence of multiple naturally occurring DENV strains. The “DENV-derived peptide” may be directly isolated from a naturally occurring DENV, for example by enzymatic cleavage of the DENV, or more typically, it may be synthesized using the amino acid sequence of a naturally occurring DENV or a consensus sequence of multiple naturally occurring DENV strains as a prototype/reference. The synthesis may be performed according to standard procedures in the art such as recombinant production techniques, genetic engineering techniques or chemical synthesis. A “DENV-derived peptide” can comprise artificial amino acids and non-natural amino acids (e.g. D-amino acids, amino acid analogues etc.). In some embodiments, a “DENV-derived peptide” may further comprise one or more conservative substitutions to the amino acid sequence originating from naturally occurring DENV strain(s). The term “ZIKV-derived peptide” is to be construed accordingly.

The term “isolated” as used herein in relation to a peptide refers to a peptide that is removed from its natural environment (e.g. a cell, tissue, culture medium, body fluid, etc.), or otherwise increased in purity to any degree (e.g. isolated from a synthesis medium). Thus, an “isolated” peptide can be naturally produced or synthetic. A peptide may be “isolated” by separating it from some or all of the substances with which it is associated in nature or with which it is associated as a result of synthetic production. Thus, an “isolated” peptide is typically at least partially purified.

The term “antigen binding protein” as used herein broadly refers to any peptide-based molecule that recognizes and binds to a target such as a virus, for example, a flavivirus such as Zika virus (ZIKV) or dengue virus (DENV).

Examples of antigen binding proteins include antibodies, including an antibody of any of the five major classes of immunoglobulins: IgA, IgD, IgE, IgG, and IgM, or subclasses (isotypes) thereof (e.g. IgG1, IgG2, IgG3, IgG4, IgA1 and IgA2), and antigen-binding fragments thereof. In some examples, the antigen binding protein comprises anti-ZIKV IgG. In some examples, the antigen binding protein comprises anti-DENV IgG.

The term “reacts with” as used herein broadly refers to the binding between an antigen-binding protein (such as an antibody or fragment thereof) and an antigen/epitope with a level higher than the binding between a non-specific antigen-binding protein (such as a non-specific antibody or fragment thereof from e.g. a healthy sample and/or a non-flavivirus infected sample and/or a non ZIKV-infected sample and/or a non DENV-infected sample) and the same antigen/epitope. For example, a sample “reacts with” a peptide if the sample comprises antigen-binding protein that shows higher binding activity for the peptide as compared to any antigen-binding protein from a control sample (e.g. a non-flavivirus-infected control sample). The term “reaction” is to be construed accordingly.

The term “response” as used herein may be understood as a measure of a binding activity between an antigen-binding protein and a peptide. The binding activity between an antigen-binding protein and a peptide may be measured by an immunoassay.

The term “recognizes more strongly” as used herein to compare a response of an antigen-binding protein for two or more peptides derived from different flaviviruses (for example, a ZIKV-derived peptide and a DENV-derived peptide) encompasses the meaning that a binding activity between the antigen-binding protein and a peptide (e.g. a ZIKV-derived peptide) is higher than a binding activity, if any, between the antigen-binding protein and another peptide (e.g. DENV-derived peptide). In some examples, an antigen-binding protein recognizes a ZIKV-derived peptide more strongly than a DENV-derived peptide if a binding activity between the antigen-binding protein and the ZIKV-derived peptide, as measured by an immunoassay, is higher than that between the antigen-binding protein and the DENV-derived peptide.

The term “immune response” as used herein encompasses both cellular and humoral immune responses. In some embodiments, the immune response is sufficient to provide immunoprotection against a flavivirus such as ZIKV or DENV.

The term “stimulate” as used herein in relation to an immune response broadly refers to an increase, an amplification or a boosting of an immune response. The term “stimulate” encompasses an initial stimulation of a new immune response or an enhancement of a pre-existing immune response.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases (such as flavivirus infections), symptoms and disorders. A medical condition also includes a body's response to a disease or disorder, e.g. inflammation. Those in need of such treatment include those already with a medical condition as well as those prone to getting the medical condition or those in whom a medical condition is to be prevented.

The term “subject” as used herein includes patients and non-patients. The term “patient” refers to individuals suffering or are likely to suffer from a medical condition such as a flavivirus infection, while “non-patients” refer to individuals not suffering and are likely to not suffer from the medical condition. “Non-patients” include healthy individuals, non-diseased individuals and/or an individual free from the medical condition. The term “subject” includes humans and animals.

Animals include murine and the like. “Murine” refers to any mammal from the family Muridae, such as mouse, rat, and the like.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a method of identifying a flavivirus infection are disclosed hereinafter.

In various embodiments, there is provided a method of identifying a flavivirus infection, the method comprising: determining whether a sample of the subject reacts with a peptide.

In various embodiments, the peptide is associated with a relative binding capacity of or capable of giving a relative binding capacity of no more than about 0.10, no more than about 0.09, no more than about 0.08, no more than about 0.07, no more than about 0.06, no more than about 0.05, no more than about 0.04, no more than about 0.03, no more than about 0.02, no more than about 0.01, less than about or equal to about 0.10, less than about or equal to about 0.09, less than about or equal to about 0.08, less than about or equal to about 0.07, less than about or equal to about 0.06, less than about or equal to about 0.05, less than about or equal to about 0.04, less than about or equal to about 0.03, less than about or equal to about 0.02 or less than about or equal to about 0.01.

In various embodiments, the peptide is associated with a relative binding capacity of or capable of giving a relative binding capacity of no less than about 0.50, no less than about 0.45, no less than about 0.40, no less than about 0.35, no less than about 0.30, no less than about 0.25, no less than about 0.20, no less than about 0.15, no less than about 0.10, no less than about 0.05, more than about or equal to about 0.50, more than about or equal to about 0.45, more than about or equal to about 0.40, more than about or equal to about 0.35, more than about or equal to about 0.30, more than about or equal to about 0.25, more than about or equal to about 0.20, more than about or equal to about 0.15, more than about or equal to about 0.10 or more than about or equal to about 0.05.

In various embodiments, the relative binding capacity is a function one or more of the following: a binding capacity of a Zika virus (ZIKV)-induced antigen binding protein, a binding capacity of a dengue virus (DENV)-induced antigen binding protein, a response of the ZIKV-induced antigen binding protein with the peptide, a response of the ZIKV-induced antigen binding protein with a corresponding peptide, a response of the DENV-induced antigen binding protein with the peptide and a response of the DENV-induced antigen binding protein with a corresponding peptide. In various embodiments, the relative binding capacity is the difference between the binding capacity of a ZIKV-induced antigen binding protein and the binding capacity of a DENV-induced antigen binding protein. In various embodiments, the relative binding capacity can be calculated by subtracting the binding capacity of a DENV-induced antigen binding protein from the binding capacity of a ZIKV-induced antigen binding protein. In various embodiments, the binding capacity of a DENV-induced antigen binding protein is the binding capacity of the DENV-induced antigen binding protein for ZIKV in relation to DENV. In some embodiments, the binding capacity of a DENV-induced antigen binding protein is calculated by: [(a response of the DENV-induced antigen binding protein with a corresponding peptide−a response of the DENV-induced antigen binding protein with the peptide)/the response of the DENV-induced antigen binding protein with the peptide]. In some embodiments, the binding capacity of a DENV-induced antigen binding protein is calculated by: [(a response of the DENV-induced antigen binding protein with the peptide−a response of the DENV-induced antigen binding protein with a corresponding peptide)/a response of the DENV-induced antigen binding protein with the corresponding peptide]. In various embodiments, the binding capacity of a ZIKV-induced antigen binding protein is the binding capacity of the ZIKV-induced antigen binding protein for ZIKV in relation to DENV. In some embodiments, the binding capacity of a ZIKV-induced antigen binding protein is calculated by: [(a response of the ZIKV-induced antigen binding protein with a corresponding peptide−a response of the ZIKV-induced antigen binding protein with the peptide)/the response of the ZIKV-induced antigen binding protein with the peptide]. In some embodiments, the binding capacity of a ZIKV-induced antigen binding protein is calculated by: [(a response of the ZIKV-induced antigen binding protein with the peptide−a response of the ZIKV-induced antigen binding protein with a corresponding peptide)/the response of the ZIKV-induced antigen binding protein with the corresponding peptide].

In various embodiments, the peptide comprises a ZIKV-derived peptide or a DENV-derived peptide. In various embodiments, where the peptide comprises a ZIKV-derived peptide, the corresponding peptide comprises a DENV-derived peptide having a sequence homologous to the peptide/ZIKV-derived peptide. In various embodiments, where the peptide comprises a DENV-derived peptide, the corresponding peptide comprises a ZIKV-derived peptide having a sequence homologous to the peptide/DENV-derived peptide.

In some examples, where the peptide comprises a ZIKV-derived peptide, the corresponding peptide comprising a DENV-derived peptide having a sequence homologous to the peptide/ZIKV-derived may be identified by aligning the sequences of ZIKV and DENV. In some examples, the homologous regions of ZIKV and DENV are at about the same amino acid positions. Thus, in some examples, the corresponding peptide comprising a DENV-derived peptide having a sequence homologous to the peptide/ZIKV-derived may be identified by aligning the sequences of ZIKV and DENV, determining the region on ZIKV that the ZIKV-derived peptide maps to, and identifying the corresponding amino acid residues at the same region or the same amino acid positions on DENV. For example, where the peptide/ZIKV-derived peptide has a sequence corresponding to the amino acids at positions 56-72 of a ZIKV prM protein (the first amino acid in the prM protein being annotated as 1), the corresponding peptide may have a sequence corresponding to the amino acids at about the same positions of a DENV prM protein i.e. positions 56-72 of the prM protein of a DENV. A corresponding peptide comprising a ZIKV-derived peptide having a sequence homologous to a DENV-derived peptide may be identified in a similar manner. The homologous sequences of ZIKV and DENV may or may not share high sequence similarity or a common evolutionary origin. In various embodiments, for a flavivirus having multiple strains e.g. DENV, the amino acid sequence may be obtained from a consensus sequence of the flavivirus e.g. DENV. As may be appreciated, a consensus sequence represents an “average” sequence in which each position represents the amino acid most often found when multiple sequences (e.g. sequences of difference strains of a flavivirus) are compared/aligned.

In various embodiments, the flavivirus infection comprises Zika virus (ZIKV) and/or dengue virus (DENV).

In various embodiments, there is provided a method of identifying a flavivirus infection selected from Zika virus (ZIKV), dengue virus (DENV) and combination thereof in a subject, the method comprising: determining whether a sample of the subject reacts with a peptide e.g. a peptide P that is capable of giving a relative binding capacity BCᵣₑₗₐₜᵢᵥₑ≤0.05 or ≥0.1, wherein

$${BC}_{relative} = \frac{{R}_{P 1 z} - {R}_{P 2 z}}{{R}_{P 2 z}} - \frac{{R}_{P 1 d} - {R}_{P 2 d}}{{R}_{P 2 d}}$$

wherein where the peptide P comprises a ZIKV-derived peptide,

R_{P1z}=response of a ZIKV-induced antigen binding protein with peptide P,

R_{P2z}=response of the ZIKV-induced antigen binding protein with a corresponding peptide C comprising a DENV-derived peptide having a sequence homologous to the peptide P,

R_{P1d}=response of a DENV-induced antigen binding protein with the peptide P,

R_{P2d}=response of the DENV-induced antigen binding protein with the corresponding peptide C comprising a DENV-derived peptide having a sequence homologous to the peptide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein ,$$

wherein where the peptide P comprises a DENV-derived peptide,

R_{P1z}=response of a ZIKV-induced antigen binding protein with a corresponding peptide C comprising a ZIKV-derived peptide having a sequence homologous to the peptide P,

R_{P2z}=response of the ZIKV-induced antigen binding protein with the peptide P,

R_{P1d}=response of a DENV-induced antigen binding protein with the corresponding peptide C comprising a ZIKV-derived peptide having a sequence homologous to the peptide P,

R_{P2d}=response of the DENV-induced antigen binding protein with the pea tide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein .$$

Without wishing to be bound by theory, it is believed that ZIKV and/or DENV antigen binding protein is generated by a subject's body in response to ZIKV and/or DENV infection and thus are present in sample from a subject having a disease state in various phases. In some embodiments, a ZIKV- and/or DENV-induced antigen binding protein comprises an antibody, optionally a serum antibody, that is generated in a subject in response to ZIKV and/or DENV infection. Examples of ZIKV-induced antigen binding protein include anti-ZIKV IgM (e.g. human anti-ZIKV IgM), anti-ZIKV IgG (e.g. human anti-ZIKV IgG1, IgG2, IgG3 and IgG4) and anti-ZIKV IgA (e.g. human anti-ZIKV IgA). Examples of DENV-induced antigen binding protein include anti-DENV IgM (e.g. human anti-DENV IgM), anti-DENV IgG (e.g. human anti-DENV IgG1, IgG2, IgG3 and IgG4) and anti-DENV IgA (e.g. human anti-DENV IgA).

The ZIKV- and/or DENV-induced antigen binding protein may be present or detectable in the subject's body at various phases of a ZIKV- and/or DENV-infection including an acute phase, an early convalescent phase, a late convalescent phase, an early recovery phase, a late recovery phase or a full recovery phase, although the levels of the ZIKV- and/or DENV-induced antigen binding protein may vary in the different phases. For example, an anti-ZIKV IgM may be detectable during an acute phase of ZIKV infection, peak at an early convalescent phase and decrease during a recovery phase. For example, an anti-ZIKV IgG may peak at an early convalescent phase, persist at high levels during late recovery and remain detectable a year after infection. Variations may also occur within the IgG isotypes. For example, an anti-ZIKV IgG1 may peak at an early convalescent phase while an anti-ZIKV IgG3 may peak at a late convalescent phase. Thus, detection of a ZIKV- and/or DENV-induced antigen binding protein, e.g. by use of a peptide as disclosed herein, may identify a ZIKV and DENV infection in a subject, and may also further identify a phase of the infection, or a prior infection.

In various embodiments, the ZIKV- and/or DENV-induced antigen binding protein is protective against ZIKV and/or DENV. In some examples, a ZIKV-induced antigen binding protein is capable of substantially neutralizing ZIKV in a neutralization assay.

In various embodiments, the binding capacity of the ZIKV-induced antigen binding protein is from about −0.50 to about 0.50, from about −0.40 to about 0.40, from about −0.30 to about 0.40, from about −0.25 to about 0.35, from about −0.21 to about 0.34, from about −0.20 to about −0.01, from about −0.10 to about −0.01, from about 0.01 to about 0.35, from about 0.01 to about 0.30, from about 0.01 to about 0.20 or from about 0.01 to about 0.10.

In various embodiments, the binding capacity of the DENV-induced antigen binding protein is from about −0.60 to about 0.30, from about −0.50 to about 0.20, from about −0.455 to about 0.173, from about −0.50 to about −0.01, from about −0.40 to about −0.01, from about −0.30 to about −0.01, from about −0.20 to about −0.01, from about −0.10 to about −0.01, from about 0.01 to about 0.30, from about 0.01 to about 0.20 or from about 0.01 to about 0.10.

In various embodiments, the ZIKV-induced antigen binding protein or the DENV-derived antigen binding protein having a binding capacity of more than 0 is indicative that the ZIKV- or DENV-induced antigen binding protein recognizes ZIKV/ZIKV-derived peptide more strongly than DENV/DENV-derived peptide and/or has higher binding activity for ZIKV/ZIKV-derived peptide than DENV/DENV-derived peptide. For example, the ZIKV- or DENV-induced antigen binding protein may give a signal/readout of stronger intensity/magnitude, e.g. a chemiluminescence signal/readout of stronger intensity with ZIKV/ZIKV-derived peptide as compared to DENV/DENV-derived peptide in an immunoassay for measuring binding activity to peptide(s).

In various embodiments, the ZIKV-induced antigen binding protein or the DENV-derived antigen binding protein having a binding capacity of less than 0 is indicative that the ZIKV- or DENV-induced antigen binding protein recognizes DENV/DENV-derived peptide more strongly than ZIKV/ZIKV-derived peptide and/or has higher binding activity for DENV/DENV-derived peptide than ZIKV/ZIKV-derived peptide. For example, the ZIKV- or DENV-induced antigen binding protein may give a signal/readout of stronger intensity/magnitude, e.g. a chemiluminescence signal/readout of stronger intensity with DENV/DENV-derived peptide as compared to ZIKV/ZIKV-derived peptide in an immunoassay for measuring binding activity to peptide(s).

In various embodiments, the peptide is capable of reacting with, or capable of being recognized by a ZIKV- and/or DENV-induced antigen binding protein (e.g. in a subject's sample) at various phases of an infection, optionally at similar intensity/strength at the various phases. In some embodiments, the peptide is capable of reacting with, or capable of being recognized by a ZIKV- and/or DENV-induced antigen binding protein only at certain phase(s) of an infection (optionally at similar intensity/strength), but not at other phase(s). In some embodiments, the peptide is capable of reacting with, or capable of being recognized by a ZIKV- and/or DENV-induced antigen binding protein at an acute phase of an infection. In some embodiments, the peptide is capable of reacting with, or capable of being recognized by a ZIKV- and/or DENV-induced antigen binding protein at a late convalescent phase of an infection and beyond (optionally at similar intensity/strength). In some embodiments, the peptide is capable of reacting with, or capable of being recognized by a ZIKV- and/or DENV-induced antigen binding protein at an acute phase and a late convalescent phase of an infection and beyond (optionally at similar intensity/strength). In some embodiments, the peptide is capable of reacting with, or capable of being recognized by a ZIKV- and/or DENV-induced antigen binding protein from an acute phase to a full recovery phase of an infection, or all phases of an infection (optionally at similar intensity/strength).

In various embodiments, the peptide comprises an epitope of ZIKV and/or DENV. In various embodiments, the peptide comprises an immunodominant epitope of ZIKV and/or DENV.

In various embodiments, the peptide comprises an antigen/epitope on a prM protein, an E glycoprotein or a NS1 protein of ZIKV or DENV. In some embodiments, the peptide comprises an epitope of a ZIKV structural protein selected from prM protein and E glycoprotein. In some embodiments, the peptide comprises an epitope of a ZIKV non-structural protein selected from NS1 protein. In some embodiments, the peptide comprises a linear antigen/epitope on the prM protein, the E glycoprotein or the NS1 protein of ZIKV or DENV.

In some embodiments, the antigen/epitope comprises exposed residues on the prM protein, the E glycoprotein or the NS1 protein. In some embodiments, the antigen/epitope is located in a solvent-exposed region of the prM protein, the E glycoprotein or the NS1 protein. Thus, the antigen/epitope may have high solvent accessibility. The antigen/epitope may be located in an accessible/exposed region of ZIKV or DENV.

In some embodiments, the antigen/epitope comprises non-exposed residues or buried residues or semi-buried residues on the prM protein, the E glycoprotein or the NS1 protein. In some embodiments, the antigen/epitope is located in a solvent-inaccessible region of the prM protein, the E glycoprotein or the NS1 protein. Thus, in some embodiments, the antigen/epitope may have low solvent accessibility. The antigen/epitope may be located in an inaccessible/buried region of ZIKV or DENV. In one example, the antigen/epitope is on the relatively inaccessible stem region of E glycoprotein. In one example. the antigen/epitope on the relatively inaccessible stem region of E glycoprotein comprises SEQ ID NO. 32 (FKSLFGGMSWFSQILIGT) or SEQ ID NO. 83 (YTALFSGVSWVMKIGIGV), or a peptide sharing at least about 70%, at least about 75%, at least about 80%, at least about 85%, at least about 90%, at least about 95%, or at least about 99% sequence identity with SEQ ID NO. 32 or 83 or portions thereof or a peptide differing by about one, about two, about three, about four, about five, about six or more amino acids with the SEQ ID NO.32 or 83 or portions thereof, or portions of SEQ ID NO.32 or 83.

Various structural data and software programs are available for evaluating an accessibility of an epitope or sequence on a protein. For example, accessibility of an epitope on E glycoprotein or NS1 protein may be evaluated based on structural data available at the Protein Data Bank. For example, accessibility of an epitope on prM protein may be predicted using T-TASSER queries together with visualization using UCSF Chimera software. As used herein, the phrase “solvent-exposed region” refers to solvent accessibility, which is in turn defined as the extent of burial or exposure of amino acid residues in the 3-dimensional protein structure. In some examples, solvent-exposed regions can be visualized using software known in the art, such as, but is not limited to, PyMOL (Schröndinger).

In various embodiments, the length/size of the peptide is no more than about 30, no more than about 29, no more than about 28, no more than about 27, no more than about 26, no more than about 25, no more than about 24, no more than about 23, no more than about 22, no more than about 21, no more than about 20, no more than about 19, no more than about 18, no more than about 17, no more than about 16, no more than about 15, no more than about 14, no more than about 13, no more than about 12, no more than about 11, no more than about 10, no more than about 9, no more than about 8, no more than about 7, no more than about 6 or no more than about 5 amino acids long. In various embodiments, the length/size of the peptide is from about 5 to about 30, from about 10 to about 25 or from about 15 to about 20 amino acids long. In one embodiment, the peptide is from about 5 to about 25 amino acids long. In some embodiments, the length/size of the peptide is about 10, about 11, about 12, about 13, about 14, about 15, about 16, about 17, about 18, about 19, about 20 or about 21 amino acids long. In various embodiments, the peptide comprises an oligopeptide. In various embodiments, the peptide comprises a short oligopeptide. In various embodiments, the peptide comprises a linear peptide.

In some embodiments, the sequence similarity/identity between the peptide and the corresponding peptide is about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or about 100%.

In some embodiments, the peptide shares a high sequence similarity/identity with the corresponding peptide. In various embodiments, the sequence similarity/identity between the peptide and the corresponding peptide is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or at least about 100%. In various embodiments, the sequence similarity/identity between the peptide and the corresponding peptide flavivirus is between about 50% to about 100%, between about 60% to about 100%, between about 70% to about 100%, between about 80% to about 100% or between about 90% to about 100%. In some embodiments, high sequence similarity/identity between the peptide and the corresponding peptide may contribute to or may be associated to each of their ability to be recognized and/or bound by both ZIKV-induced antigen binding protein and DENV-induced antigen binding protein. In some embodiments, the ZIKV-induced antigen binding protein and the DENV-induced antigen binding protein may recognize and/or bind to the peptide and/or the corresponding peptide with substantially equal intensity/strength.

In some embodiments, the peptide and/or the corresponding peptide is capable of being recognized and/or bound by both ZIKV-induced antigen binding protein and DENV-induced antigen binding protein. In some embodiments, the peptide and/or the corresponding peptide is capable of reacting with both ZIKV-induced antigen binding protein and DENV-induced antigen binding protein. In some embodiments, the ZIKV-induced antigen binding protein and the DENV-induced antigen binding protein may recognize and/or bind to and/or react with the peptide and/or the corresponding peptide with substantially equal intensity/strength.

Thus, advantageously, embodiments of the peptide may serve as a marker, e.g. a serology marker, for identifying a flavivirus infection from other infections, or for identifying either of a ZIKV or a DENV infection or for distinguishing a ZIKV or a DENV infection from other flavivirus infection (e.g. yellow fever virus (YFV), Japanese encephalitis virus, West Nile encephalitis virus, St. Louis encephalitis virus, tick-borne encephalitis virus, Kyasanur forest disease virus and Alkhurma hemorrhagic fever virus etc.).

In some embodiments, the peptide shares a low sequence similarity/identity with the corresponding peptide. In various embodiments, the sequence similarity/identity between the peptide and the corresponding peptide is no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70% or no more than about 75%. In various embodiments, the sequence similarity/identity between the peptide and the corresponding peptide is between about 5% to about 75%, between about 5% to about 50%, between about 15% to about 75% or between about 15% to about 50%. In some embodiments, low sequence similarity/identity between the peptide and the corresponding peptide may contribute to or may be associated to each of their ability to be differentially recognized and/or bound by ZIKV-induced antigen binding protein and DENV-induced antigen binding protein. Hence, there may be less cross-reactivity between a ZIKV-induced antigen binding protein and a DENV-induced antigen binding protein for the same peptide or corresponding peptide and accordingly, this improve the differentiation performance in flavivirus detection e.g. between ZIKV and DENV.

In some embodiments, the peptide shares no more than about 50% sequence similarity with the corresponding peptide.

In some embodiments, the peptide and/or the corresponding peptide is capable of being specifically recognized and/or specifically bound by a ZIKV-induced antigen binding protein but not a DENV-induced antigen binding protein. In some embodiments, the peptide and/or the corresponding peptide is capable of reacting with a ZIKV-induced antigen binding protein but not a DENV-induced antigen binding protein. Thus, advantageously, embodiments of the peptide may serve as a marker, e.g. a serology marker, for identifying ZIKV infection and/or for distinguishing a ZIKV infection from a DENV infection or from other flavivirus infection.

In some embodiments, the peptide and/or the corresponding peptide is capable of being specifically recognized and/or specifically bound by a DENV-induced antigen binding protein but not a ZIKV-induced antigen binding protein. In some embodiments, the peptide and/or the corresponding peptide is capable of reacting with a DENV-induced antigen binding protein but not a ZIKV-induced antigen binding protein. Thus, advantageously, embodiments of the peptide may serve as a marker, e.g. a serology marker, for identifying DENV infection and/or for distinguishing a DENV infection from a ZIKV infection or from other flavivirus infection.

In various embodiments, the peptide is selected from a peptide in Table 8, a peptide sharing at least about 70%, at least about 75%, at least about 80%, at least about 85%, at least about 90%, at least about 95%, or at least about 99% sequence identity with the peptide or portions thereof, a peptide differing by about one, about two, about three, about four, about five, about six or more amino acids with the peptide or portions thereof, or portions of the peptide.

In various embodiments, the peptide comprises: a sequence corresponding to the amino acid sequence at the following positions on a ZIKV and/or DENV prM protein (the first amino acid in the prM protein being annotated as 1): from about 3 to about 26, from about 6 to about 23, from about 12 to about 35, from about 15 to about 32, from about 21 to about 44, from about 24 to about 41, from about 30 to about 53, from about 33 to about 50, from about 39 to about 62, from about 42 to about 59, from about 48 to about 71, from about 51 to about 68, from about 53 to about 75, from about 56 to about 72, from about 66 to about 89, from about 69 to about 86, from about 75 to about 95, from about 78 to about 92, from about 97 to about 121 or from about 100 to about 118. In various embodiments, the peptide comprises a peptide sharing at least about 70%, at least about 75%, at least about 80%, at least about 85%, at least about 90%, at least about 95%, or at least about 99% sequence identity with the amino acid sequences or portions thereof, or a peptide differing by about one, about two, about three, about four, about five, about six or more amino acids with the amino acid sequences or portions thereof, or portions of the amino acid sequences.

In various embodiments, the peptide is selected from the group consisting of: SEQ ID NO. 1 (RGSAYYMYLDRNDAGEAI), SEQ ID NO. 52 (RDGEPRMIVGKNERGKSL), SEQ ID NO. 2 (DRNDAGEAISFPTTLGMN), SEQ ID NO. 53 (GKNERGKSLLFKTASGIN), SEQ ID NO. 3 SFPTTLGMNKCYIQIMDL), SEQ ID NO. 54 (LFKTASGINMCTLIAMDL), SEQ ID NO. 4 (KCYIQIMDLGHMCDATMS), SEQ ID NO. 55 (MCTLIAMDLGEMCDDTVT), SEQ ID NO. 5 (GHMCDATMSYECPMLDEG), SEQ ID NO. 56 (GEMCDDTVTYKCPHITE), SEQ ID NO. 6 (YECPMLDEGVEPDDVDCW), SEQ ID NO. 57 (YKCPHITEVEPEDIDCW), SEQ ID NO. 7 (LDEGVEPDDVDCWCNTT), SEQ ID NO. 58 (HITEVEPEDIDCWCNLT), SEQ ID NO. 8 (CNTTSTWVVYGTCHHKKG), SEQ ID NO. 59 (CNLTSTWVTYGTCNQAG), SEQ ID NO. 9 (YGTCHHKKGEARRSR), SEQ ID NO. 60 (TSTWVTYGTCNQAG), SEQ ID NO. 10 (HSTRKLQTRSQTWLESREY), SEQ ID NO. 61 (VGMGLDTRTQTWMSAEGAW), a peptide sharing at least about 70%, at least about 75%, at least about 80%, at least about 85%, at least about 90%, at least about 95%, or at least about 99% sequence identity with the sequences or portions thereof, a peptide differing by about one, about two, about three, about four, about five, about six or more amino acids with the sequences or portions thereof, or portions of the sequences.

In various embodiments, the peptide comprises: a sequence corresponding to the amino acid sequence at the following positions on a ZIKV and/or DENV E glycoprotein (the first amino acid in the E glycoprotein being annotated as 1): from about 34 to about 57, from about 37 to about 54, from about 58 to about 75, from about 61 to about 72, from about 70 to about 93, from about 73 to about 90, from about 88 to about 111, from about 91 to about 108, from about 110 to about 133, from about 113 to about 130, from about 120 to about 143, from about 123 to about 140, from about 128 to about 152, from about 131 to about 149, from about 146 to about 169, from about 149 to about 166, from about 154 to about 177, from about 157 to about 174, from about 163 to about 186, from about 166 to about 183, from about 188 to about 206, from about 191 to about 203, from about 196 to about 219, from about 199 to about 216, from about 214 to about 237, from about 217 to about 234, from about 232 to about 248, from about 235 to about 245, from about 241 to about 264, from about 244 to about 261, from about 268 to about 291, from about 271 to about 288, from about 303 to about 322, from about 306 to about 319, from about 322 to about 345, from about 325 to about 342, from about 340 to about 358, from about 343 to about 355, from about 358 to about 381, from about 361 to about 378, from about 399 to about 422, from about 402 to about 419, from about 450 to about 473 or from about 453 to about 470. In various embodiments, the peptide comprises a peptide sharing at least about 70%, at least about 75%, at least about 80%, at least about 85%, at least about 90%, at least about 95%, or at least about 99% sequence identity with the amino acid sequences or portions thereof, or a peptide differing by about one, about two, about three, about four, about five, about six or more amino acids with the amino acid sequences or portions thereof, or portions of the amino acid sequences.

In various embodiments, the peptide is selected from the group consisting of: SEQ ID NO. 11 (DKPTVDIELVTTTVSNMA), SEQ ID NO. 62 (NKPTLDIELQKTEATQLA), SEQ ID NO. 12 (YEASISDMASDS), SEQ ID NO. 63 (IEGKITNITTDS), SEQ ID NO. 13 (RCPTQGEAYLDKQSDTQY), SEQ ID NO. 64 (RCPTQGEAVLPEEQDQNY), SEQ ID NO. 14 (VCKRTLVDRGWGNGCGLF), SEQ ID NO. 65 (VCKHTYVDRGWGNGCGLF), SEQ ID NO. 15 (LVTCAKFACSKKMTGKSI), SEQ ID NO. 66 (LVTCAKFQCLEPIEGKVV), SEQ ID NO. 16 (KKMTGKSIQPENLEYRIM), SEQ ID NO. 67 (EPIEGKVVQYENLKYTVI), SEQ ID NO. 17 (PENLEYRIMLSVHGSQHS), SEQ ID NO. 68 (YENLKYTVIITVHTGDQH), SEQ ID NO. 18 (SGMIVNDTGHETDENRAK), SEQ ID NO. 69 (GDQHQVGNETQGVTAEIT), SEQ ID NO. 19 (GHETDENRAKVEITPNSP), SEQ ID NO. 70 (GNETQGVTAEITPQASTT), SEQ ID NO. 20 (KVEITPNSPRAEATLGGF), SEQ ID NO. 71 (TAEITPQASTTEAILPEY), SEQ ID NO. 21 (EPRTGLDFSDLYY), SEQ ID NO. 72 (SPRTGLDFNEMIL), SEQ ID NO. 22 (SDLYYLTMNNKHWLVHKE), SEQ ID NO. 73 (NEMILLTMKNKAWMVHRQ), SEQ ID NO. 23 (WFHDIPLPWHAGADTGTP), SEQ ID NO. 74 (WFFDLPLPWTSGATTETP), SEQ ID NO. 24 (HWNNKEALVEF), SEQ ID NO. 75 (TWNRKELLVTF), SEQ ID NO. 25 (EFKDAHAKRQTVVVLGSQ), SEQ ID NO. 76 (TFKNAHAKKQEVVVLGSQ), SEQ ID NO. 26 (GALEAEMDGAKGRLSSGH), SEQ ID NO. 77 (GATEIQNSGGTSIFAGH), SEQ ID NO. 27 (SLCTAAFTFTKIPA), SEQ ID NO. 78 (AMCTNTFVLKKEVS), SEQ ID NO. 28 (TVTVEVQYAGTDGPCKVP), SEQ ID NO. 79 (TILIKVEYKGEDAPCKIP), SEQ ID NO. 29 (AQMAVDMQTLTPV), SEQ ID NO. 80 (FSTEDGQGKAHN), SEQ ID NO. 30 (ANPVITESTENSKMMLEL), SEQ ID NO. 81 (ANPVVTKKEEPVNIEA), SEQ ID NO. 31 (RSGSTIGKAFEATVRGAK), SEQ ID NO. 82 (KKGSSIGKMFEATARGAR), SEQ ID NO. 32 (FKSLFGGMSWFSQILIGT), SEQ ID NO. 83 (YTALFSGVSWVMKIGIGV), a peptide sharing at least about 70%, at least about 75%, at least about 80%, at least about 85%, at least about 90%, at least about 95%, or at least about 99% sequence identity with the sequences or portions thereof, a peptide differing by about one, about two, about three, about four, about five, about six or more amino acids with the sequences or portions thereof, or portions of the sequences.

In various embodiments, the peptide comprises: a sequence corresponding to the amino acid sequence at the following positions on a ZIKV and/or DENV NS1 protein (the first amino acid in the NS1 protein being annotated as 1): from about 1 to about 21, from about 1 to about 18, from about 16 to about 39, from about 19 to about 36, from about 52 to about 75, from about 55 to about 72, from about 67 to about 88, from about 70 to about 85, from about 88 to about 115, from about 91 to about 112, from about 116 to about 139, from about 119 to about 136, from about 134 to about 157, from about 137 to about 154, from about 152 to about 179, from about 155 to about 176, from about 236 to about 259, from about 239 to about 256, from about 245 to about 268, from about 248 to about 265, from about 254 to about 277, from about 257 to about 274, from about 267 to about 284, from about 270 to about 281, from about 272 to about 295, from about 275 to about 292, from about 281 to about 304, from about 284 to about 301, from about 290 to about 313, from about 293 to about 310, from about 299 to about 322, from about 302 to about 319, from about 312 to about 329, from about 315 to about 326, from about 317 to about 340, from about 320 to about 337, from about 335 to about 356, or from about 338 to about 353. In various embodiments, the peptide comprises a peptide sharing at least about 70%, at least about 75%, at least about 80%, at least about 85%, at least about 90%, at least about 95%, or at least about 99% sequence identity with the amino acid sequences or portions thereof, or a peptide differing by about one, about two, about three, about four, about five, about six or more amino acids with the amino acid sequences or portions thereof, or portions of the amino acid sequences.

In various embodiments, the peptide is selected from the group consisting of: SEQ ID NO. 33 (DVGCSVDFSKKETRCGTG), SEQ ID NO. 84 (DMGCVINWKGKELKCGSG), SEQ ID NO. 34 (VFVYNDVEAWRDRYKYHP), SEQ ID NO. 85 (IFVTNEVHTWTEQYKFQA), SEQ ID NO. 35 (CGISSVSRMENIMWRSVE), SEQ ID NO. 86 (CGIRSTTRMENLLWKQIA), SEQ ID NO. 36 (SVEGELNAILEENGVQ), SEQ ID NO. 87 (QIANELNYILWENNIK), SEQ ID NO. 37 (GSVKNPMWRGPQRLPVPVNELP), SEQ ID NO. 88 (GDIIGVLEQGKRTLTPQPMELK), SEQ ID NO. 38 (GKSYFVRAAKTNNSFVVD), SEQ ID NO. 89 (GKAKIVTAETQNSSFIID), SEQ ID NO. 39 (GDTLKECPLKHRAWNSFL), SEQ ID NO. 90 (GPNTPECPSASRAWNVWE), SEQ ID NO. 40 (VEDHGFGVFHTSVWLKVREDYS), SEQ ID NO. 91 (VEDYGFGVFTTNIWLKLREVYT), SEQ ID NO. 41 (SDLIIPKSLAGPLSHHNT), SEQ ID NO. 92 (SDMIIPKSLAGPISQHNH), SEQ ID NO. 42 (AGPLSHHNTREGYRTQMK), SEQ ID NO. 93 (AGPISQHNHRPGYHTQTA), SEQ ID NO. 43 (REGYRTQMKGPWHSEELE), SEQ ID NO. 94 (RPGYHTQTAGPWHLGKLE), SEQ ID NO. 44 (SEELEIRFEECP), SEQ ID NO. 95 (LGKLELDFNYCE), SEQ ID NO. 45 (IRFEECPGTKVHVEETCG), SEQ ID NO. 96 (LDFNYCEGTTWITENCG), SEQ ID NO. 46 (KVHVEETCGTRGPSLRST), SEQ ID NO. 97 (TVVITENCGTRGPSLRTT), SEQ ID NO. 47 (TRGPSLRSTTASGRVIEE), SEQ ID NO. 98 (TRGPSLRTTTVSGKLIHE), SEQ ID NO. 48 (TASGRVIEEWCCRECTMP), SEQ ID NO. 99 (TVSGKLIHEWCCRSCTLP), SEQ ID NO. 49 (ECTMPPLSFRAK), SEQ ID NO. 100 (SCTLPPLRYMGE), SEQ ID NO. 50 (PLSFRAKDGCWYGMEIRP), SEQ ID NO. 101 (PLRYMGEDGCWYGMEIRP), SEQ ID NO. 51 (RKEPESNLVRSMVTAG), SEQ ID NO. 102 (ISEKEENMVKSLVSAG), a peptide sharing at least about 70%, at least about 75%, at least about 80%, at least about 85%, at least about 90%, at least about 95%, or at least about 99% sequence identity with the sequences or portions thereof, a peptide differing by about one, about two, about three, about four, about five, about six or more amino acids with the sequences or portions thereof, or portions of the sequences.

In various embodiments, the peptide comprises one or more ZIKV-derived peptide selected from the group consisting of: SEQ ID NO: 7 (LDEGVEPDDVDCWCNTT); SEQ ID NO: 36 (SVEGELNAILEENGVQ); SEQ ID NO: 38 (GKSYFVRAAKTNNSFWD); SEQ ID NO: 39 (GDTLKECPLKHRAWNSFL); SEQ ID NO: 46 (KVHVEETCGTRGPSLRST); SEQ ID NO: 49 (ECTMPPLSFRAK); SEQ ID NO: 3 (SFPTTLGMNKCYIQIMDL); SEQ ID NO: 26 (GALEAEMDGAKGRLSSGH); SEQ ID NO: 32 (FKSLFGGMSWFSQILIGT); SEQ ID NO: 9 (YGTCHHKKGEARRSR); SEQ ID NO: 17 (PENLEYRIMLSVHGSQHS); SEQ ID NO: 43 (REGYRTQMKGPWHSEELE); SEQ ID NO: 45 (IRFEECPGTKVHVEETCG); a peptide sharing at least 75% sequence identity thereto; or a peptide differing by one, two, three or four amino acids thereto; or portions thereof; and/or one or more DENV-derived peptide selected from the group consisting of SEQ ID NO: 58 (HITEVEPEDIDCVWCNLT); SEQ ID NO: 87 (QIANELNYILWENNIK); SEQ ID NO: 89 (GKAKIVTAETQNSSFIID); SEQ ID NO: 90 (GPNTPECPSASRAWNVWE); SEQ ID NO: 97 (TWITENCGTRGPSLRTT); SEQ ID NO: 100 (SCTLPPLRYMGE); SEQ ID NO: 54 (LFKTASGINMCTLIAMDL); SEQ ID NO: 77 (GATEIQNSGGTSIFAGH); SEQ ID NO: 83 (YTALFSGVSWVMKIGIGV); SEQ ID NO: 60 (TSTVWTYGTCNQAG); SEQ ID NO: 68 (YENLKYTVIITVHTGDQH); SEQ ID NO: 94 (RPGYHTQTAGPWHLGKLE); SEQ ID NO: 96 (LDFNYCEGTTWITENCG); or a peptide sharing at least 75% sequence identity thereto; or a peptide differing by one, two, three or four amino acids thereto; or portions thereof.

Embodiments of the method may identify a flavivirus infection, one or both of ZIKV and DENV infections, a ZIKV infection or a DENV infection.

In various embodiments, the method comprises determining whether the sample reacts with one or more peptide (e.g. a common flavivirus peptide) selected from the group consisting of SEQ ID NO: 7 (LDEGVEPDDVDCVWCNTT); SEQ ID NO: 36 (SVEGELNAILEENGVQ); SEQ ID NO: 38 (GKSYFVRAAKTNNSFWD); SEQ ID NO: 39 (GDTLKECPLKHRAWNSFL); SEQ ID NO: 46 (KVHVEETCGTRGPSLRST); SEQ ID NO: 49 (ECTMPPLSFRAK); SEQ ID NO: 58 (HITEVEPEDIDCVWCNLT); SEQ ID NO: 87 (QIANELNYILWENNIK); SEQ ID NO: 89 (GKAKIVTAETQNSSFIID); SEQ ID NO: 90 (GPNTPECPSASRAWNVWE); SEQ ID NO: 97 (TVVITENCGTRGPSLRTT); SEQ ID NO: 100 (SCTLPPLRYMGE); a peptide sharing at least 75% sequence identity thereto; or a peptide differing by one, two, three or four amino acids thereto; or portions thereof; and/or wherein where the sample reacts with the one or more peptide or the portions thereof, the subject is indicated for flavivirus infection or indicated for one or both of ZIKV and DENV infections.

In various embodiments, the method is a method of identifying a flavivirus infection in an acute phase, and the method comprises determining whether the sample reacts with one or more peptide selected from the group consisting of: SEQ ID NO: 7 (LDEGVEPDDVDCWCNTT); SEQ ID NO: 36 (SVEGELNAILEENGVQ); SEQ ID NO: 38 (GKSYFVRAAKTNNSFWD); SEQ ID NO: 58 (HITEVEPEDIDCWCNLT); SEQ ID NO: 87 (QIANELNYILWENNIK); SEQ ID NO: 89 (GKAKIVTAETQNSSFIID), wherein where the sample reacts with the one or more peptide or the portions thereof, the subject is indicated for flavivirus infection or indicated for one or both of ZIKV and DENV infections in an acute phase.

In various embodiments, the method comprises determining whether the sample reacts with one or more peptide (e.g. a ZIKV-specific peptide) selected from the group consisting of: SEQ ID NO: 3 (SFPTTLGMNKCYIQIMDL); SEQ ID NO: 26 (GALEAEMDGAKGRLSSGH); SEQ ID NO: 32 (FKSLFGGMSWFSQILIGT); a peptide sharing at least 75% sequence identity thereto; or a peptide differing by one, two, three or four amino acids thereto; or portions thereof; wherein where the sample reacts with the one or more peptide or the portions thereof, the subject is indicated for ZIKV infection.

In various embodiments, the method is a method of identifying a ZIKV infection in an acute phase, and the method comprises determining whether the sample reacts with: SEQ ID NO: 32 (FKSLFGGMSWFSQILIGT); a peptide sharing at least 75% sequence identity thereto; or a peptide differing by one, two, three or four amino acids thereto; or portions thereof; wherein where the sample reacts with the peptide or the portions thereof, the subject is indicated for ZIKV infection in an acute phase.

In various embodiments, the method comprises determining whether the sample reacts with one or more peptide (e.g. a DENV-specific peptide) selected from the group consisting of SEQ ID NO: 60 (TSTWVTYGTCNQAG); SEQ ID NO: 68 (YENLKYTVIITVHTGDQH); SEQ ID NO: 94 (RPGYHTQTAGPWHLGKLE); SEQ ID NO: 96 (LDFNYCEGTTVVITENCG); and/or a peptide sharing at least 75% sequence identity thereto; or a peptide differing by one, two, three or four amino acids thereto; or portions thereof; wherein where the sample reacts with the one or more peptide or the portions thereof, the subject is indicated for DENV infection.

Embodiments of the method may comprise use of multiple or a plurality of the peptides as described herein. In some embodiments, at least about two, at least about three, at least about four or at least about five distinct peptides may be used for detecting ZIKV- and/or DENV-induced antigen binding protein in a sample. In some embodiments, the plurality of the peptides comprises at least one peptide from one or more of the following groups: a common flavivirus peptide, a ZIKV-specific peptide and a DENV-specific peptide.

In various embodiments, determining whether a sample of the subject reacts to a peptide comprises performing an immunoassay to assess whether antigen binding proteins (e.g. antibodies) that are capable of recognizing or binding to the peptide are present in the sample. Examples of immunoassay include radioimmunoassay (RIA), chemiluminescence- and fluorescence-immunoassays, enzyme-linked immunosorbent assay (ELISA), enzyme-linked immunosorbent spot (ELISPOT), Luminex-based bead arrays, protein microarray assays, immunochromatography (ICG)-based assay and rapid test formats such as immunochromatographic strip tests. The assay can be competitive and non-competitive sandwich assays.

In some embodiments, the immunoassay comprises ELISA, such as, but is not limited to direct ELISA, indirect ELISA, competitive ELISA, sandwich ELISA, and the like. In some embodiments, the immunoassay comprises a sandwich ELISA. The suitability of the specific ELISA to be used would be within the purview of the skill of the person skilled in the art. However, as an illustration of the present invention, in some embodiments, the assay is in the form of a non-competitive sandwich assay, wherein a ZIKV and/or DENV antibody or a fragment thereof to be detected and/or quantified is bound to an antibody of the ZIKV and/or DENV antibody or fragment thereof and/or a ZIKV and/or DENV antigen. In some examples, a ZIKV and/or DENV antigen may be labelled, for example with biotin, and may be bound to a solid phase e.g. a bead, a surface of a well or other containment body, a chip or a strip, for capturing any ZIKV and/or DENV antibody or a fragment thereof, and an antibody of the ZIKV and/or DENV antibody is used for detecting any captured ZIKV and/or DENV antibody. The detection antibody may be labelled, for example, with a dye, radioisotope or a reactive or catalytically active moiety. In one example, the detection antibody is labelled with horseradish peroxidase (HRP) and tetramethylbenzidine (TMB) substrate may be added for visualization. It will be appreciated that other suitable labels and substrates may also be used. In some examples, a plate (e.g. microplate, microtiter plate etc.) coated with streptavidin is used for capturing the biotin-labelled ZIKV and/or DENV antigen, which is then used for capturing any ZIKV and/or DENV antibody or a fragment thereof, and a labelled anti-immunoglobulin is used for detection. The anti-immunoglobulin may be anti-IgG, anti-IgM or anti-IgA. In some examples, the anti-immunoglobulin comprises anti-human IgM, anti-human IgG, anti-human IgG1, anti-human IgG2, anti-human IgG3 and/or anti-human IgG4.

In various embodiments, the sample comprises a biological sample. In various embodiments, the biological sample comprises a fluid biological sample or a liquid biological sample. The fluid biological sample or liquid biological sample may be blood, serum, plasma, sputum, lavage fluid, cerebrospinal fluid, urine, semen, sweat, tears, saliva, and the like. In some embodiments, the fluid biological sample or liquid biological sample comprises whole blood, blood serum, blood plasma or processed fractions thereof. In some embodiments, the fluid biological sample comprises blood serum or blood plasma. In some embodiments, the fluid biological sample comprises antigen binding proteins such as antibodies.

In various embodiments, the sample comprises a sample that is collected from a subject during an acute phase (about 2 days to about 7 days post illness onset (pio)), an early convalescent phase (about 10 days to about 14 days pio), a late convalescent phase (about 1 month pio), an early recovery phase (about 3 months pio), a late recovery phase (about 5 months to about 6 months pio) or a full recovery phase (about 1 year pio). In some embodiments, the sample is collected from a subject when subject shows symptoms associated with ZIKV infection, such as fever, arthritis/arthralgia, skin rash, conjunctivitis, joint pain, headache, Guillain-Barré syndrome (GBS) and congenital central nervous system (CNS) abnormalities. In some embodiments, the sample is collected from a subject when subject is or has become asymptomatic for ZIKV infection. In some embodiments, the sample is collected from a subject when subject shows symptoms associated with DENV infection, such as fever (typically high fever), headache, muscle, bone, and joint pain, nausea, vomiting, pain behind the eyes, swollen glands, rash, severe abdominal pain, persistent vomiting, bleeding from gums or nose, blood in urine, stools or vomit, bleeding under the skin, difficult or rapid breathing, cold or clammy skin (shock), fatigue, and irritability or restlessness. In some embodiments, the sample is collected from a subject when subject is or has become asymptomatic for DENV infection.

In various embodiments, the method comprises a diagnostic method or a prognostic method. For example, a reaction of a subject's sample to the peptide may be indicative of ZIKV and/or DENV infection in the subject. For example, a reduced level of reaction of the subject's sample relative to an earlier sample (e.g. a sample collected from the same subject at an earlier time point) may be indicative of prognosis of ZIKV and/or DENV infection in the subject.

In various embodiments, the method has high sensitivity and/or specificity. In various embodiments, the method has a sensitivity of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100%.

In various embodiments, the method has a specificity of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100%.

In various embodiments, the method comprises an in vitro or an ex vivo method.

In some embodiments, the method may identify/distinguish a flavivirus infection from other infections, e.g. bacterial infections, other viral infections or other non-flavivirus infections. In some embodiments, the method may identify/distinguish a flavivirus infection from other infections sharing similar symptom(s) with a flavivirus infection. For example, the method may identify/distinguish a flavivirus infection from influenza, Chikungunya infection, malaria, typhoid fever and so on. In some embodiments, the method may distinguish ZIKV infection and/or DENV infection from other flavivirus infection. In some embodiments, the method may distinguish ZIKV infection from DENV infection, and/or DENV infection from ZIKV infection.

In some embodiments, there is provided a method of distinguishing a flavivirus infection from other infections in a subject, the method comprising determining whether a sample of the subject reacts with a peptide that is capable of giving a relative binding capacity BCᵣₑₗₐₜᵢᵥₑ≤0.05. In some embodiments, there is provided a method of distinguishing ZIKV and/or DENV infection from other flavivirus infection in a subject, the method comprising determining whether a sample of the subject reacts with a peptide that is capable of giving a relative binding capacity BCᵣₑₗₐₜᵢᵥₑ≤0.05. In some embodiments, there is provided a method of distinguishing ZIKV infection from DENV infection, and/or DENV infection from ZIKV infection, in a subject, the method comprising determining whether a sample of the subject reacts with a peptide that is capable of giving a relative binding capacity BCᵣₑₗₐₜᵢᵥₑ≥0.1.

In some embodiments, there is provided a method of distinguishing ZIKV infection from DENV infection in a subject, the method comprising: determining whether a sample of the subject reacts with a peptide of SEQ ID NO: 32 (FKSLFGGMSWFSQILIGT); a peptide sharing at least 75% sequence identity thereto; or a peptide differing by one, two, three or four amino acids thereto; or portions thereof, wherein where the sample reacts with the peptide or the portions thereof, the subject is indicated for ZIKV infection; and/or determining whether a sample of the subject reacts with a peptide of SEQ ID NO: 9 (YGTCHHKKGEARRSR); a peptide sharing at least 75% sequence identity thereto; or a peptide differing by one, two, three or four amino acids thereto; or portions thereof, wherein where the sample reacts with the peptide or the portions thereof, the subject is indicated for DENV infection.

In some embodiments, the method further comprises administering to the subject a ZIKV and/or DENV treatment regimen if the subject is indicated for ZIKV and/or DENV infection.

In some embodiments, the method further comprises administering to the subject a ZIKV and/or DENV treatment regimen if the subject is indicated for flavivirus infection, ZIKV infection and/or DENV infection. In some embodiments therefore, there is provided a method of treating a flavivirus infection, a ZIKV infection and/or DENV infection, the method comprising determining whether a sample of the subject reacts with the peptide, wherein if the sample reacts to the peptide, administering to subject a ZIKV and/or DENV treatment regimen.

In some embodiments, there is provided a method of identifying a common flavivirus antigen/epitope, a ZIKV-specific antigen/epitope and/or DENV specific antigen/epitope, the method comprising contacting a test peptide and its corresponding peptide with a ZIKV-induced antigen binding protein and a DENV-induced antigen binding protein under conditions suitable for binding, and determining a relative binding capacity, wherein if the relative binding capacity is ≤0.05, the test peptide is identified as a common flavivirus antigen/epitope, wherein if the relative binding capacity is ≥0.1 the test peptide is identified as a ZIKV-specific antigen/epitope or a DENV specific antigen/epitope.

In some embodiments, the method further comprises generating a library of short linear peptides, optionally short overlapping linear peptides, from the flavivirus e.g. ZIKV and/or DENV for use as the test peptides and/or the corresponding peptides. In some embodiments, the method comprises an ELISA method.

In various embodiments, there is provided a kit for identifying a flavivirus infection selected from Zika virus, dengue virus and combination thereof in a subject, the kit comprising one or more peptides as disclosed herein. In some embodiments, there is provided a kit for identifying a flavivirus infection selected from Zika virus, dengue virus and combination thereof in a subject, the kit comprising a peptide that is capable of giving a relative binding capacity BCᵣₑₗₐₜᵢᵥₑ≤0.05 or ≥0.1, wherein

$${BC}_{relative} = \frac{{R}_{P 1 z} - {R}_{P 2 z}}{{R}_{P 2 z}} - \frac{{R}_{P 1 d} - {R}_{P 2 d}}{{R}_{P 2 d}}$$

wherein where the peptide P comprises a ZIKV-derived peptide,

R_{P1z}=response of a ZIKV-induced antigen binding protein with peptide P,

R_{P2z}=response of the ZIKV-induced antigen binding protein with a corresponding peptide C comprising a DENV-derived peptide having a sequence homologous to the peptide P,

R_{P1d}=response of a DENV-induced antigen binding protein with the peptide P,

R_{P2d}=response of the DENV-induced antigen binding protein with the corresponding peptide C comprising a DENV-derived peptide having a sequence homologous to the peptide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein ,$$

wherein where the peptide P comprises a DENV-derived peptide,

R_{P1z}=response of a ZIKV-induced antigen binding protein with a corresponding peptide C comprising a ZIKV-derived peptide having a sequence homologous to the peptide P,

R_{P2z}=response of the ZIKV-induced antigen binding protein with the peptide P,

R_{P1d}=response of a DENV-induced antigen binding protein with the corresponding peptide C comprising a ZIKV-derived peptide having a sequence homologous to the peptide P,

R_{P2d}=response of the DENV-induced antigen binding protein with the peptide P,

$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein .$$

In various embodiments of the kit, the peptide comprises one or more ZIKV-derived peptide selected from the group consisting of: SEQ ID NO: 7 (LDEGVEPDDVDCVWCNTT); SEQ ID NO: 36 (SVEGELNAILEENGVQ); SEQ ID NO: 38 (GKSYFVRAAKTNNSFWD); SEQ ID NO: 39 (GDTLKECPLKHRAWNSFL); SEQ ID NO: 46 (KVHVEETCGTRGPSLRST); SEQ ID NO: 49 (ECTMPPLSFRAK); SEQ ID NO: 3 (SFPTTLGMNKCYIQIMDL); SEQ ID NO: 26 (GALEAEMDGAKGRLSSGH); SEQ ID NO: 32 (FKSLFGGMSWFSQILIGT); SEQ ID NO: 9 (YGTCHHKKGEARRSR); SEQ ID NO: 17 (PENLEYRIMLSVHGSQHS); SEQ ID NO: 43 (REGYRTQMKGPWHSEELE); SEQ ID NO: 45 (IRFEECPGTKVHVEETCG); a peptide sharing at least 75% sequence identity thereto; or a peptide differing by one, two, three or four amino acids thereto; or portions thereof; and/or one or more DENV-derived peptide selected from the group consisting of SEQ ID NO: 58 (HITEVEPEDIDCVWCNLT); SEQ ID NO: 87 (QIANELNYILWENNIK); SEQ ID NO: 89 (GKAKIVTAETQNSSFIID); SEQ ID NO: 90 (GPNTPECPSASRAWNVWE); SEQ ID NO: 97 (TWITENCGTRGPSLRTT); SEQ ID NO: 100 (SCTLPPLRYMGE); SEQ ID NO: 54 (LFKTASGINMCTLIAMDL); SEQ ID NO: 77 (GATEIQNSGGTSIFAGH); SEQ ID NO: 83 (YTALFSGVSVWMKIGIGV); SEQ ID NO: 60 (TSTVWTYGTCNQAG); SEQ ID NO: 68 (YENLKYTVIITVHTGDQH); SEQ ID NO: 94 (RPGYHTQTAGPWHLGKLE); SEQ ID NO: 96 (LDFNYCEGTTWITENCG); or a peptide sharing at least 75% sequence identity thereto; or a peptide differing by one, two, three or four amino acids thereto; or portions thereof.

The kit may also further comprise one or more of the following: a plate coated with a capture agent for anti-ZIKV and/or anti-DENV, and a detection agent for detecting the presence of captured anti-ZIKV and/or anti-DENV. The capture and detection agent may be independently selected from a purified ZIKV and/or DENV, ZIKV and/or DENV antigen, antibody of anti-ZIKV and/or anti-DENV or an anti-immunoglobulin. The anti-immunoglobulin may be one or more of anti-IgA, anti-IgM, anti-IgG, anti-IgG1, anti-IgG2, anti-IgG3 and anti-IgG4, anti-human IgA, anti-human IgM, anti-human IgG, anti-human IgG1, anti-human IgG2, anti-human IgG3 and anti-human IgG4. In some embodiments, the capture agent comprises ZIKV and/or DENV antigen and the detection agent comprises a labelled anti-immunoglobulin. In some embodiments, the capture agent comprises anti-immunoglobulin and the detection agent comprises labelled ZIKV and/or DENV antigen. In some embodiments, the capture agent and the detection agent comprise a ZIKV and/or DENV antigen and/or an anti-immunoglobulin. In some embodiments, the capture agent and the detection agent are selected from the group consisting of (i) a ZIKV and/or DENV antigen and one or more of a labelled anti-immunoglobulin and (ii) an anti-immunoglobulin and a labelled ZIKV and/or DENV antigen.

In some embodiments, the kit further comprises one or more of the following: a plate coated with a capture agent for anti-ZIKV and/or anti-DENV, and a detection agent for detecting the presence of captured anti-ZIKV and/or anti-DENV, wherein the capture agent and the detection agent comprise a ZIKV and/or DENV antigen and/or an anti-immunoglobulin.

The kit may be a diagnostic kit or a prognostic kit.

In various embodiments, the subject comprises a mammal. In various embodiments, the subject comprises a human subject. In various embodiments, the subject comprises an Asian subject. In some embodiments, the subject comprises a Southeast Asian subject. In various embodiments, the subject (whether or not a Southeast Asian subject) is infected with flavivirus in Asia, or optionally in Southeast Asian. Southeast Asian countries include Indonesia, Malaysia, Singapore, Philippines, East Timor, Brunei, Cambodia, Laos, Myanmar (Burma), Thailand and Vietnam.

The ZIKV may be an African or Asian strain, has an African or Asian genotype or it may be of African or Asian lineage. In some embodiments, the ZIKV comprises an Asian strain, optionally a Southeast Asian strain. In some embodiments, the ZIKV comprises an Asian genotype, optionally a Southeast Asian genotype. In some embodiments, the ZIKV comprises a ZIKV of Asian lineage, optionally of Southeast Asian lineage. Non-limiting examples of an Asian ZIKV strain/ZIKV having Asian genotype/ZIKV of Asian lineage include H/PF/2013 (KJ776791), SPH2015, PRVABC59, R103451, P6-740 and FSS 13025. Non-limiting examples of an African ZIKV strain/ZIKV of African genotype/ZIKV of African lineage include MR-766 and DakAr41524.

The DENV may be of serotype 1 (DENV-1), 2 (DENV-2), 3 (DENV-3) or 4 (DENV-4), or a consensus sequence of one or more serotypes. DENV-1 may be of genotypes I, II, III (sylvatic), IV, V, and VI. DENV-2 may be of Asian-I, Asian-II, Asian/American, American, Cosmopolitan, and sylvatic. DENV-3 may be of genotypes I, II, III, IV, and V. DENV-4 may be of genotypes I, IIA, IIB, III, and sylvatic. In some embodiments, the DENV comprises a DENV of a strain/genotype/lineage found in and/or originating from and/or predominant in an Asian country, optionally a Southeast Asian country. Non-limiting examples of such DENV include strains KR296743, KF973487, EU081181, KF041254, JF808120, JF808121, KJ189293, KC762692, KC425219, KJ830751, KF973479, and AY099336.

In various embodiments, there is provided a peptide, optionally an isolated peptide, comprising a peptide as disclosed herein. In various embodiments, there is provided a peptide, optionally an isolated peptide, selected from the group consisting of SEQ ID NO: 7 (LDEGVEPDDVDCWCNTT); SEQ ID NO: 36 (SVEGELNAILEENGVQ); SEQ ID NO: 38 (GKSYFVRAAKTNNSFWD); SEQ ID NO: 39 (GDTLKECPLKHRAWNSFL); SEQ ID NO: 46 (KVHVEETCGTRGPSLRST); SEQ ID NO: 49 (ECTMPPLSFRAK); SEQ ID NO: 3 (SFPTTLGMNKCYIQIMDL); SEQ ID NO: 26 (GALEAEMDGAKGRLSSGH); SEQ ID NO: 32 (FKSLFGGMSWFSQILIGT); SEQ ID NO: 9 (YGTCHHKKGEARRSR); SEQ ID NO: 17 (PENLEYRIMLSVHGSQHS); SEQ ID NO: 43 (REGYRTQMKGPWHSEELE); SEQ ID NO: 45 (IRFEECPGTKVHVEETCG); SEQ ID NO: 58 (HITEVEPEDIDCWCNLT); SEQ ID NO: 87 (QIANELNYILWENNIK); SEQ ID NO: 89 (GKAKIVTAETQNSSFIID); SEQ ID NO: 90 (GPNTPECPSASRAWNVWE); SEQ ID NO: 97 (TVVITENCGTRGPSLRTT); SEQ ID NO: 100 (SCTLPPLRYMGE); SEQ ID NO: 54 (LFKTASGINMCTLIAMDL); SEQ ID NO: 77 (GATEIQNSGGTSIFAGH); SEQ ID NO: 83 (YTALFSGVSWVMKIGIGV); SEQ ID NO: 60 (TSTWVTYGTCNQAG); SEQ ID NO: 68 (YENLKYTVIITVHTGDQH); SEQ ID NO: 94 (RPGYHTQTAGPWHLGKLE); SEQ ID NO: 96 (LDFNYCEGTTWITENCG); or a peptide sharing at least 75% sequence identity thereto; or a peptide differing by one, two, three or four amino acids thereto; or portions thereof.

In various embodiments, there is provided a composition, optionally an immune system modulating composition, further optionally an immune system stimulating/activating composition, further optionally a vaccine composition, comprising a peptide as disclosed herein. The composition may be capable of stimulating, e.g. increasing, amplifying or boosting, an immune response against flavivirus and/or ZIKV and/or DENV infection in a subject when administered to the subject. In various embodiments, the composition is capable of increasing an immune response against flavivirus and/or ZIKV and/or DENV infection in a subject by at least about 10%, at least about 20%, at least about 30%, at least about 40%, at least about 50%, at least about 60%, at least about 70%, at least about 80%, at least about 90%, at least about 100%, at least about 2-fold, at least about 3-fold, at least about 4-fold, at least about 5-fold, at least about 6-fold, at least about 7-fold, at least about 8-fold, at least about 9-fold or at least about 10-fold after administration to the subject. In some examples, an immune response of the subject can be determined by measuring an amount of ZIKV- and/or DENV-antigen binding proteins in a sample from the subject. The composition may further comprise an adjuvant. Examples of suitable adjuvants include aluminium hydroxide and aluminium phosphate which may enable a body's immune response to be increased when administered.

In some embodiments, there is provided a composition, optionally an immune modulating composition, further optionally an immune system stimulating/activating composition, further optionally a vaccine composition comprising a peptide selected from the group consisting of: SEQ ID NO: 7 (LDEGVEPDDVDCVWCNTT); SEQ ID NO: 36 (SVEGELNAILEENGVQ); SEQ ID NO: 38 (GKSYFVRAAKTNNSFWD); SEQ ID NO: 39 (GDTLKECPLKHRAWNSFL); SEQ ID NO: 46 (KVHVEETCGTRGPSLRST); SEQ ID NO: 49 (ECTMPPLSFRAK); SEQ ID NO: 3 (SFPTTLGMNKCYIQIMDL); SEQ ID NO: 26 (GALEAEMDGAKGRLSSGH); SEQ ID NO: 32 (FKSLFGGMSWFSQILIGT); SEQ ID NO: 9 (YGTCHHKKGEARRSR); SEQ ID NO: 17 (PENLEYRIMLSVHGSQHS); SEQ ID NO: 43 (REGYRTQMKGPWHSEELE); SEQ ID NO: 45 (IRFEECPGTKVHVEETCG); SEQ ID NO: 58 (HITEVEPEDIDCVWCNLT); SEQ ID NO: 87 (QIANELNYILWENNIK); SEQ ID NO: 89 (GKAKIVTAETQNSSFIID); SEQ ID NO: 90 (GPNTPECPSASRAWNVWE); SEQ ID NO: 97 (TWITENCGTRGPSLRTT); SEQ ID NO: 100 (SCTLPPLRYMGE); SEQ ID NO: 54 (LFKTASGINMCTLIAMDL); SEQ ID NO: 77 (GATEIQNSGGTSIFAGH); SEQ ID NO: 83 (YTALFSGVSVWMKIGIGV); SEQ ID NO: 60 (TSTWVTYGTCNQAG); SEQ ID NO: 68 (YENLKYTVIITVHTGDQH); SEQ ID NO: 94 (RPGYHTQTAGPWHLGKLE); SEQ ID NO: 96 (LDFNYCEGTTWITENCG); or a peptide sharing at least 75% sequence identity thereto; or a peptide differing by one, two, three or four amino acids thereto; or portions thereof.

In various embodiments, there is provided a method of inducing an immune response in a subject, the method comprising administering the composition, optionally an immune modulating composition, further optionally an immune system stimulating/activating composition, further optionally a vaccine composition to the subject.

In various embodiments, there is provided a method, a kit, a peptide or a composition as described herein.

### BRIEF DESCRIPTION OF FIGURES

FIG. 1. Antibody profiles of healthy controls and ZIKV patients of Singapore cohort in 2016. Total (a) IgM and (b) IgG antibody titres of 45 healthy donors were determined by virion-based ELISA using purified ZIKV, DENV or CHIKV virions and plasma dilution of 1:2000. Data are presented as mean, with dotted line indicating the respective mean+SD values. Samples with OD values less than mean+SD for both IgM and IgG, and across all three viruses are highlighted in black (n=22), and were combined together to form the pooled healthy control for subsequent experiments. Samples with OD values more than the cut-off are denoted as clear symbols (n=23). (c) Total anti-ZIKV IgM and IgG of ZIKV patients were determined at plasma dilutions of 1:200, 1:1000, 1:2000, 1:4000 and 1:8000 in virion-based ELISA using purified ZIKV virions at time points of acute (n=58), early convalescent (n=43), late convalescent (n=45), early recovery (n=41), late recovery (n=38), and full recovery (n=32). Pooled plasma of healthy donors was used as negative control. Data are presented as mean±SEM. (d-e) Total anti-DENV (d) IgM and (e) IgG antibody titres in plasma samples of ZIKV patients at first collection time point were determined by virion-based ELISA using purified DENV virions in the same method as (c). Data are presented as mean±SEM. All ELISA readings were in duplicates. (f) Number and percentage of patients that were positive or negative for anti-DENV IgM and IgG at the first collection of plasma specimen (1:200 and 1:2000 dilutions respectively)

FIG. 2 Antibody profiles of ZIKV patients of Singapore cohort in 2016 over time. (a-c) Total anti-ZIKV (a) IgM and (b) IgG antibody titres in patients' plasma samples, at dilutions 1:200 and 1:2000 respectively, were determined by virion-based ELISA using purified ZIKV virions. Pooled plasma of healthy donors was used as negative control. Data are presented as mean±SEM, with dotted line indicating mean of pooled healthy control. (c) Number and percentage of patients that are positive or negative for anti-ZIKV IgM and IgG at the respective time points. (d) IgG isotype titres in patients' plasma samples were determined at 1:200 dilution in a ZIKV virion-based ELISA. Data are presented as mean±SEM, with dotted line indicating mean of pooled healthy control. All ELISA readings were conducted in duplicates or triplicates. [Acute (n=58), early convalescent (n=43), late convalescent (n=45), early recovery (n=41), late recovery (n=38), full recovery (n=32)]. (e-f) In vitro neutralising capacity of pooled ZIKV patients and pooled healthy control were tested at 1:1000 plasma dilution via flow cytometry. (e) Plasma samples were pooled according to levels of anti-ZIKV IgG titre [group of low titre patients are denoted as square symbol, while group of high titres are denoted as triangle symbol as shown in (b)] for acute [low (n=37), high (n=21)], early convalescent [low (n=29), high (n=14)], and late convalescent [low (n=28), high (n=17)] time points. (f) Plasma samples collected at the recovery phases were pooled together at the respective time points [early recovery (n=41), late recovery (n=38), full recovery (n=32)]. Results are expressed as percentage of control infection. Data presented as mean±SD and representative of 2 independent experiments. Statistical analysis between virus only and pooled healthy or patient samples was carried out using Mann-Whitney U test, two-tailed, with Bonferroni correction for multiple testing (*P<0.05).

FIG. 3. Neutralising capacities of antibodies of ZIKV patients against ZIKV and DENV. In vitro neutralising capacity of pooled ZIKV patients against (a) ZIKV and (b) DENV were tested at 1:500, 1:1000 and 1:2000 plasma dilutions via flow cytometry. Plasma samples were pooled according to levels of anti-ZIKV IgG titre (shown in FIG. 2) for acute [low (n=37), high (n=21)], early convalescent [low (n=29), high (n=14)], and late convalescent [low (n=28), high (n=17)] time points. Plasma samples collected at the recovery phases were pooled together at the respective time points [early recovery (n=41), late recovery (n=38), full recovery (n=32)]. Plasma of pooled healthy was used as negative control. Results are expressed as percentage of control infection. Data presented as mean±SD and representative of 2 independent experiments.

FIG. 4. Preliminary mapping of common flavivirus, ZIKV-specific, and DENV-specific linear B-cell pooled epitopes within ZIKV and DENV proteome using ZIKV and DENV patient samples. (a) Polyprotein of ZIKV H/PF/2013 (UniProtKB accession: A0A024B7W1), matched to results of pooled peptide-based ELISA experiments. Plasma samples of ZIKV patients (n=30) and serum samples of DENV patients (n=20) at 1:2000 dilution in duplicates were subjected to peptide-based ELISA using pooled peptides covering the precursor of membrane (prM; pools 1-4), envelope (E; pools 5-17) and nonstructural 1 (NS1; pools 18-25) proteins of ZIKV and DENV proteome. Each pool consists of 5 peptides of 18-mer length, with overlapping sequence of 10 amino acids. IgG response of patients were normalised to mean of pooled healthy control. Patients' response to ZIKV and DENV pooled peptide-pairs were compared and the mean binding capacity are presented in a heat-map. A value of 0 on the scale denotes patients showing equal binding response to a ZIKV and DENV pooled peptide-pair, whereas values larger than 0 show preferential of patients to bind to ZIKV pooled peptide. Values smaller than 0 show binding preference of patients to DENV pooled peptide. (b) Percentage recognition of ZIKV and DENV patients to peptide pools were calculated. (c) A schematic representation to denote potential common flavivirus, ZIKV-specific, and DENV-specific pools across prM, E and NS1 based on the heat-map analysis above.

FIG. 5. Mapping of common flavivirus, ZIKV-specific, and DENV-specific linear B cell epitopes using ZIKV and DENV patient samples. (a) Polyprotein of ZIKV H/PF/2013 (UniProtKB accession: A0A024B7W1). Plasma samples of ZIKV patients (n=30-44) and serum samples of DENV (n=20) patients at late convalescent phase were tested at 1:2000 dilution in a peptide-based ELISA in duplicates, using peptides that cover the precursor of membrane (prM: peptides 1-10), envelope (E; peptides 11-32) and non-structural 1 (NS1; peptides 33-51) proteins of ZIKV and DENV proteome. IgG response of patients were normalised to mean of pooled healthy control. Patients' response to ZIKV and DENV peptide-pairs were compared and the mean binding capacity are presented in a heat-map. A value of 0 on the scale denotes patients showing equal binding response to a ZIKV and DENV peptide-pair, whereas values larger than 0 show preferential of patients to bind to ZIKV peptide. Values smaller than 0 show binding preference of patients to DENV peptide. (b) A schematic representation to denote common flavivirus, ZIKV-specific, and DENV-specific peptides across prM, E and NS1 based on heat-map analysis above. (c-e) Genome organisation of ZIKV prM, E and NS1. Regions of amino acids corresponding to the identified linear B-cell epitopes in (c) prM, (d) E and (e) NS1 are shown. Numbers in boxes denote the peptide number, and the amino acid position in the respective proteome.

FIG. 6. Peptide binding capacity of ZIKV and DENV patients on potential common flavivirus, ZIKV-specific, and DENV-specific linear B-cell epitopes. Plasma samples of ZIKV (n=30-44) and serum samples of DENV (n=20) patients at late convalescent phase were tested at 1:2000 dilution in duplicates in a peptide-based ELISA, with pooled plasma of healthy donors used as negative control. The IgG binding capacity of patients positive for respective ZIKV and DENV peptide-pairs were calculated as [(ZIKV peptide response-DENV peptide response)/DENV peptide response] and the mean±SEM values are presented in (a) for potential common flavivirus, ZIKV-specific, and DENV-specific linear B-cell epitopes. The distribution of binding capacity of individual ZIKV and DENV patients are shown in (b) for common flavivirus, (c) for ZIKV-specific and (d) for DENV-specific peptides. Data presented as mean±SD. Statistical analysis was carried out using Mann-Whitney U test, two-tailed, with Bonferroni correction for multiple testing (**P<0.01).

FIG. 7. Characterisation of the antibody profile kinetics of ZIKV patients on common flavivirus and ZIKV-specific linear B-cell epitopes, and localisation of potential epitopes within the ZIKV and DENV proteome. (a-b) Plasma samples of ZIKV patients (n=27) at acute, late convalescent and full recovery phases were tested for IgG at 1:2000 dilution in duplicates using ZIKV and DENV peptides in a peptide-based ELISA. Pooled plasma of healthy donors was used as negative control and patients' data were normalised to mean of pooled healthy control. (a) Percentage of ZIKV patients positively binding to ZIKV and DENV peptides, and (b) binding capacity of ZIKV patients positively binding to peptides were calculated and presented in a heat-map. (c-e) Schematic diagrams showing the localisation of common flavivirus, ZIKV-specific, and DENV-specific epitopes on (c) prM protein of ZIKV and DENV (PDB: 3C6E), (d) E glycoprotein of ZIKV (PDB: 5JHM) and DENV (PDB: 1UZG), (e) stem-transmembrane (TM) domain of E glycoprotein of ZIKV (PDB: 51Z7) and DENV (PDB: 3J2P), and (f) NS1 protein of ZIKV (PDB: 5K6K) and DENV (PDB: 406B).

FIG. 8. Preliminary diagnostic validation of identified linear B-cell epitopes with patient cohorts. Convalescent plasma samples of ZIKV (n=10) and serum samples of DENV (n=10) patients from Singapore, and DENV (n=5), bacteria (n=5) and unknown (n=8) patients from Thailand were tested in a peptide-based ELISA in duplicates at 1:2000 dilution. Pooled healthy plasma was used as a negative control. (a) Sensitivity and specificity were determined for individual peptides. (b) Sensitivity and specificity of peptide mix of selected epitopes were determined. (c) Principal component analysis (PCA) of pooled healthy and patients' anti-IgG peptide response (OD values) were plotted in a graph with the percentage of variance indicated. (d-e) The peptide binding capacity of patients positively binding to peptides were calculated and statistically analysed by using Kruskal-Wallis tests with Bonferroni correction for multiple testing. Post hoc tests were done using Dunn's multiple comparison tests to determine (d) peptides with discriminating power, and (e) the peptide binding capacity distribution of patients. Data are presented as mean±SD. (*P<0.05, **P<0.01).

FIG. 9. Correlation analysis of antibody and peptide response. (a) Plasma samples of ZIKV patients (n=65) were pooled according to the levels of anti-ZIKV IgG titre (shown in FIG. 2b). Mean neutralising capacity of pooled ZIKV patients (shown in FIG. 2e-f) and mean anti-ZIKV IgG levels of ZIKV patients were calculated for all time points (from acute to full recovery) and correlation was carried out. (b-d) Plasma samples from ZIKV (n=30-44) and serum samples from DENV (n=20) patients at late convalescent time point were tested for anti-ZIKV or anti-DENV IgG respectively at 1:2000 dilution, using purified ZIKV or DENV virions in a virion-based ELISA. In addition, plasma/serum samples were tested at 1:2000 dilution in duplicates for peptide-specific IgG using ZIKV and DENV peptides in a peptide-based ELISA. Patients' response to ZIKV and DENV peptide-pairs were compared and peptide binding capacity was calculated. Correlation of (b) both ZIKV and DENV patients' antibody response to common flavivirus peptides, (c) ZIKV patients' antibody response to ZIKV-specific peptides, and (d) DENV patients' antibody response to DENV-specific peptides. Correlation analysis was carried out using Spearman's rank correlation. Spearman's rho (ρ) and P-value (P) are presented.

### EXAMPLES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments.

### ZIKV Patients Produce a Robust and Protective Humoral Response

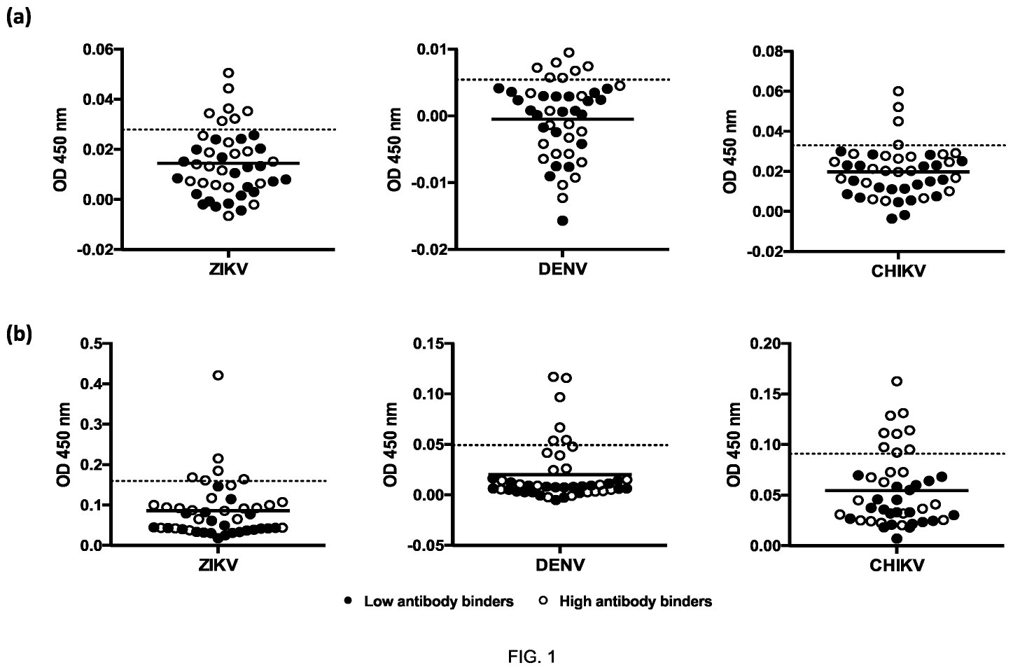

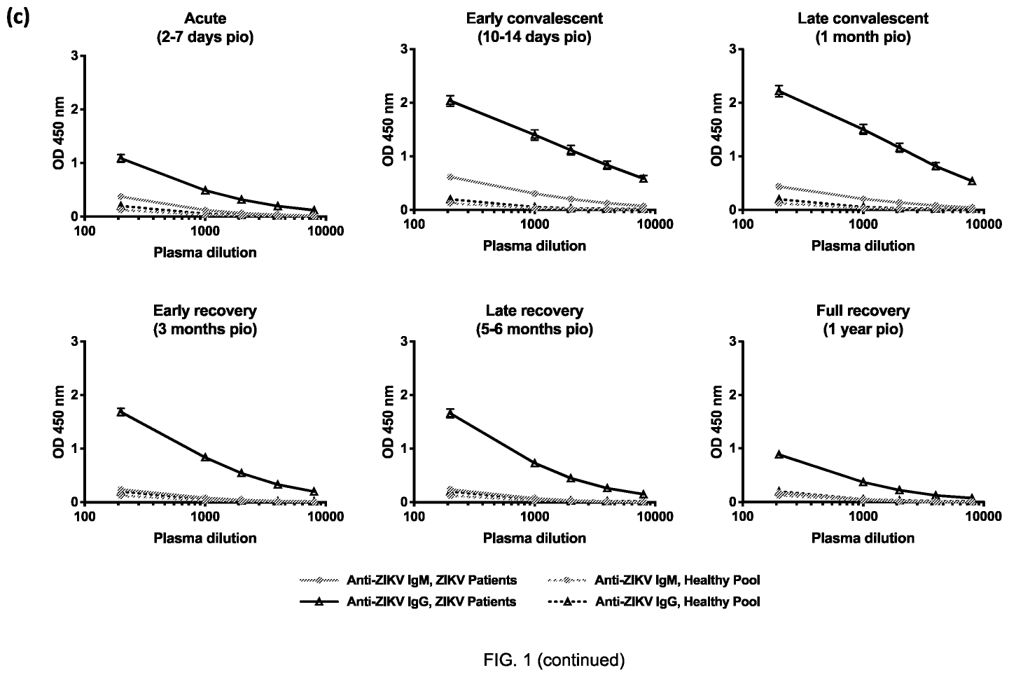

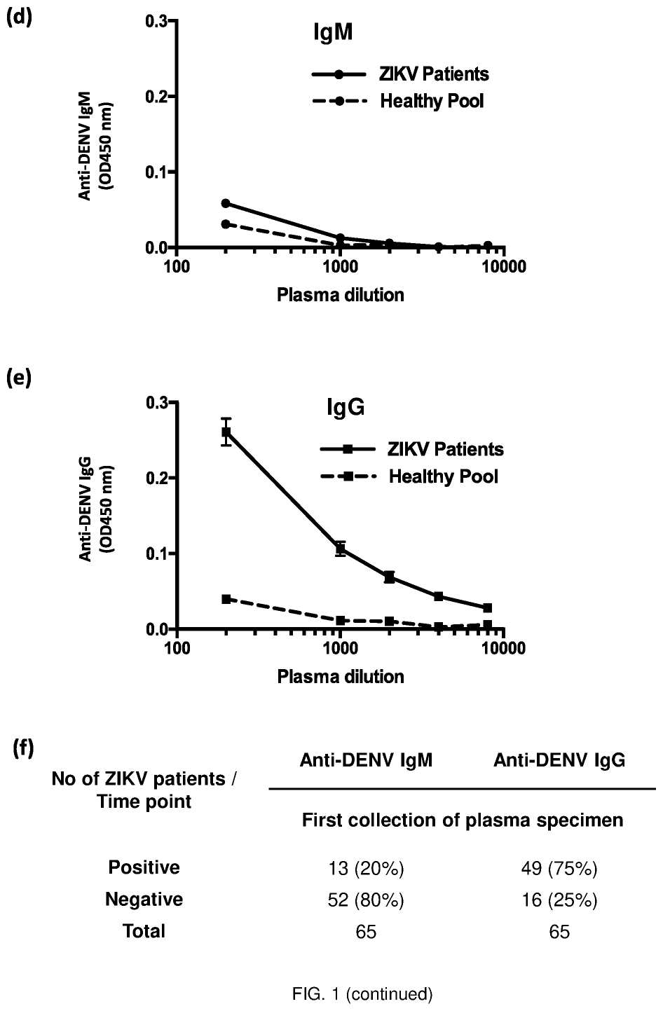

Forty-five healthy donors were first screened for the presence of IgM and IgG against ZIKV, DENV and chikungunya virus (CHIKV), the three main arboviruses co-circulating in Singapore and several parts of Asia using virion-based ELISA. Twenty-two donors which had antibody levels lower than the assigned cut-off (mean+SD) in all three viruses (FIG. 1a-b) were used as the healthy control pool, and set as a baseline reference.

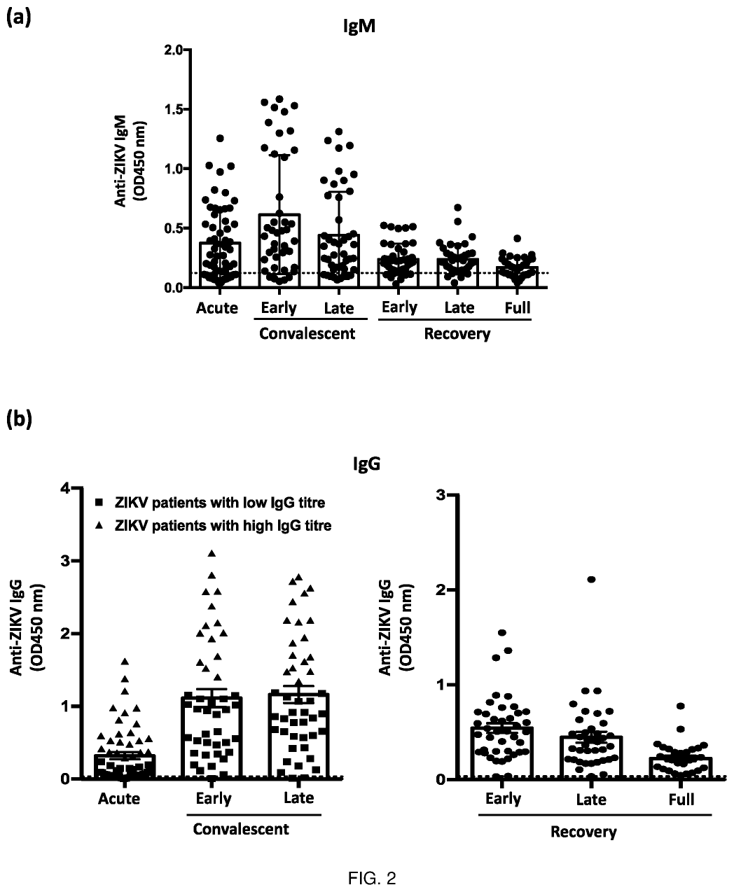

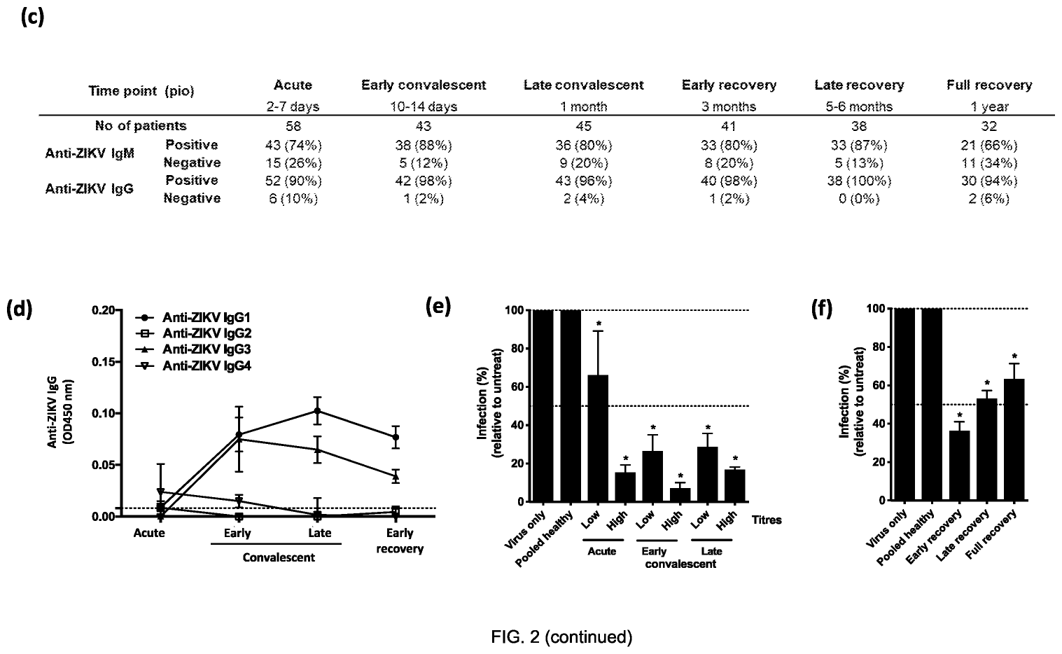

Anti-ZIKV IgM and IgG levels of ZIKV patients from the Singapore outbreak in 2016 were longitudinally assessed using virion-based ELISA. Majority of the patients showed a robust ZIKV-specific humoral response FIG. 2a-c, FIG. 1c). Anti-ZIKV IgM was detected as early as in the acute phase (2-7 days post-illness onset, pio) and peaked at early convalescent (10-14 days pio), before decreasing during the recovery phases (3 months to 1 year pio) (FIGS. 2a and c, FIG. 1c). ZIKV-specific IgG titres peaked at early convalescent, persisted at high levels during late recovery, and were still detectable a year after infection (FIG. 2b-c, FIG. 1c). These patients were also screened for the presence of DENV-specific antibodies and 80% of the patients were negative for anti-DENV IgM in samples taken at the acute phase (FIGS. 1d and 1f). However, 75% of the patients were found to have anti-DENV IgG (FIG. 1e-f), suggesting that ZIKV IgG, but not IgM, cross-reacts with DENV.

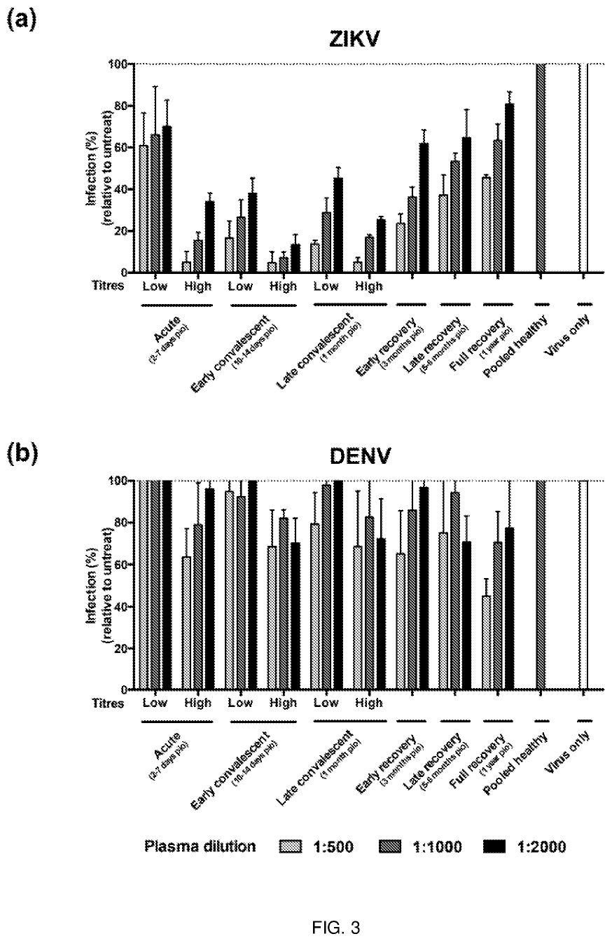

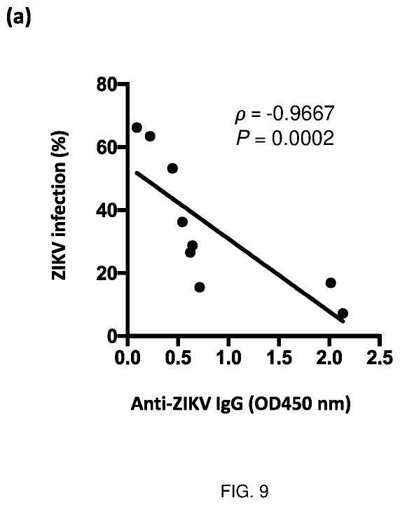

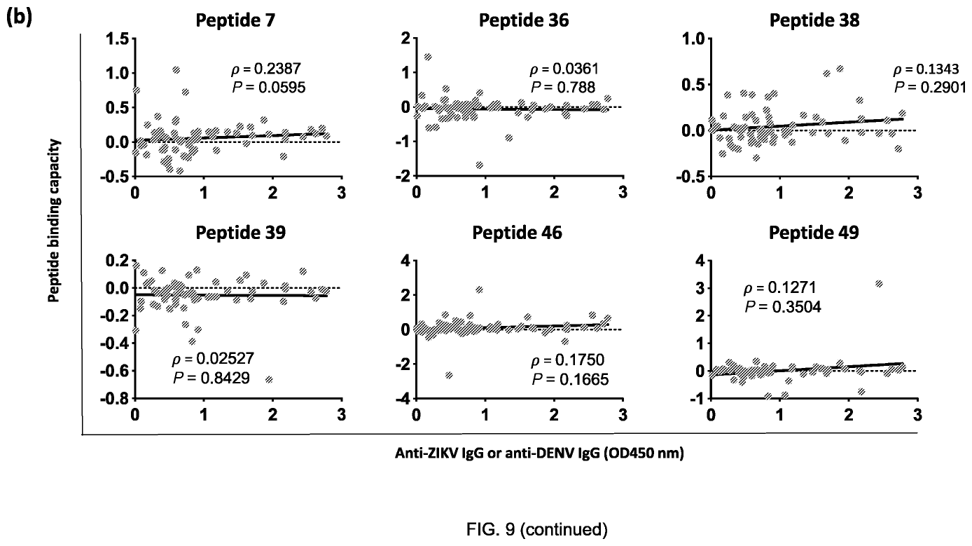

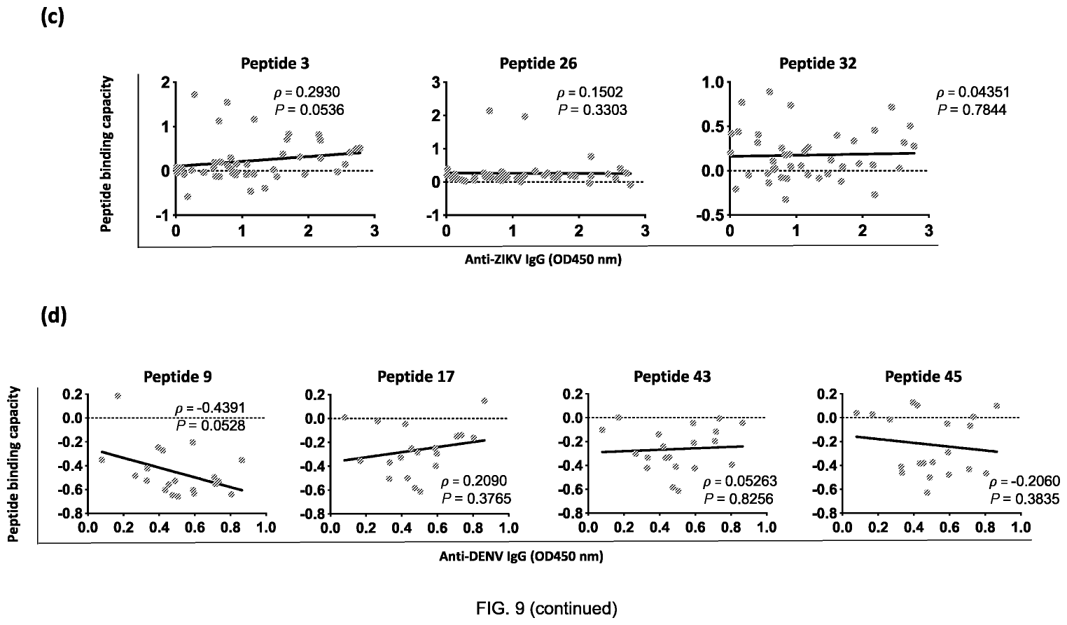

IgG isotypes produced by ZIKV patients were then determined and highest titres of anti-ZIKV IgG1 and IgG3 subtypes were produced at early convalescent for IgG3, and late convalescent for IgG1 (FIG. 2d). To determine if antibodies produced in these patients were protective against ZIKV, neutralisation assays were carried out via flow cytometry (FIG. 2e-f, FIG. 3a). Efficient neutralisation (71% to 93%) was observed in early and late convalescent stages (FIG. 2e), whilst weak neutralisation (37% to 47%) was seen in late and full recovery stages (FIG. 2f). Neutralisation capacity of ZIKV patients correlated with levels of anti-ZIKV IgG (FIG. 1c and FIG. 9a). Plasma from these patients only minimally neutralised DENV (FIG. 3b), indicating ZIKV-specificity.

Identification of Specific B-Cell Linear Epitopes Recognised by Antibodies from ZIKV and DENV Patients

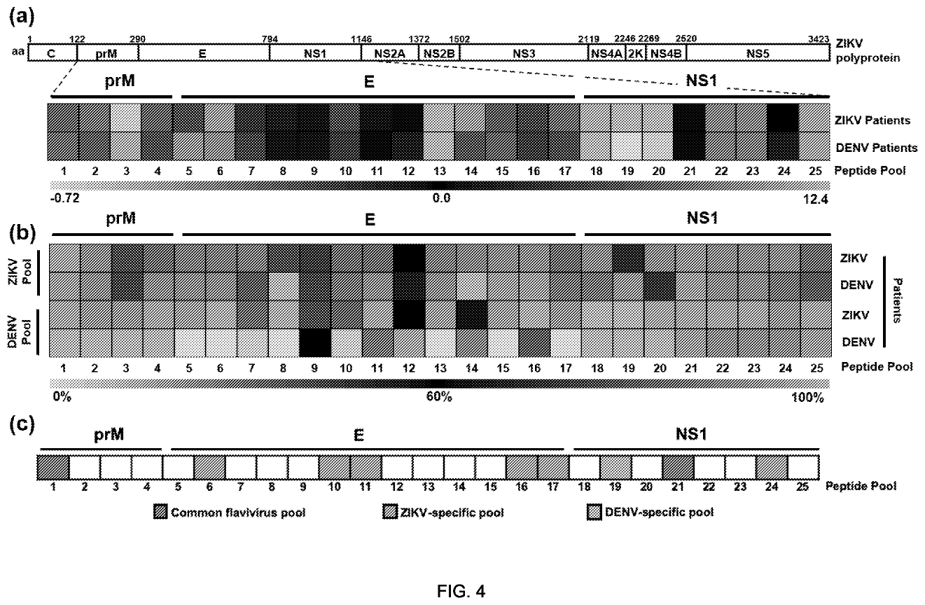

Preliminary mapping of specific ZIKV and DENV epitopes was first performed in a peptide-based ELISA on the most antigenic flavivirus antigens: prM, E and NS1, using pooled linear ZIKV and consensus DENV peptides. Plasma/serum samples of ZIKV and DENV patients taken at the late convalescent phase were used as IgG levels were highest at this time point (FIG. 1c). Results specifically showed two common flavivirus (pools 1 and 21), six potential ZIKV-specific (pools 6, 10, 11, 16, 17 and 24) and one potential DENV-specific (pool 19) pools were identified within the ZIKV and DENV proteome (Table 1, FIG. 4).

TABLE 1
Preliminary results of ZIKV and DENV patients response to ZIKV and DENV pooled peptides

| | | Percentage recognition (%)^{†} | | | | | | | |
|---|---|---|---|---|---|---|---|---|---|
| | | ZIKV Patients | | DENV Patients | | Mean binding | | | Preliminary |
| | | (n = 30) | | (n = 20) | | capacity^{‡} | | | classification |
| | Peptide | ZIKV | DENV | ZIKV | DENV | ZIKV | DENV | Relative | of peptide |
| Protein | Pool | Pool | Pool | Pool | Pool | Patients | Patients | difference^{§} | pool^{¶} |
| prM | 1 | 96.7 | 96.7 | 95 | 100 | −0.325 | −0.360 | 0.035 | Common |
| | 2 | 90.0 | 93.3 | 90 | 100 | −0.392 | −0.295 | 0.097 | |
| | 3 | 76.7 | 93.3 | 75 | 100 | −0.684 | −0.553 | 0.131 | |
| | 4 | 83.3 | 96.7 | 90 | 100 | −0.313 | −0.254 | 0.059 | |
| E | 5 | 86.7 | 16.7 | 90 | 0 | 5.066 | 10.343 | 5.277 | |
| | 6 | 86.7 | 13.3 | 90 | 0 | 10.379 | 9.512 | 0.867 | ZIKV-specific |
| | 7 | 86.7 | 30.0 | 80 | 5 | 3.102 | 4.222 | 1.120 | |
| | 8 | 40.0 | 13.3 | 10 | 0 | 1.212 | 0.741 | 0.471 | |
| | 9 | 73.3 | 76.7 | 75 | 60 | −0.029 | 0.653 | 0.681 | |
| | 10 | 83.3 | 33.3 | 85 | 5 | 4.730 | 4.043 | 0.687 | ZIKV-specific |
| | 11 | 83.3 | 96.7 | 90 | 85 | 0.763 | 0.301 | 0.462 | ZIKV-specific |
| | 12 | 60.0 | 60.0 | 65 | 15 | 0.267 | 1.222 | 0.954 | |
| | 13 | 86.7 | 16.7 | 90 | 0 | 12.259 | 12.399 | 0.140 | |
| | 14 | 23.3 | 56.7 | 10 | 25 | −0.513 | −0.279 | 0.234 | |
| | 15 | 90.0 | 16.7 | 95 | 0 | 6.344 | 6.814 | 0.470 | |
| | 16 | 90.0 | 13.3 | 95 | 35 | 4.429 | 4.040 | 0.388 | ZIKV-specific |
| | 17 | 83.3 | 20.0 | 85 | 0 | 7.079 | 5.210 | 1.869 | ZIKV-specific |
| NS1 | 18 | 90.0 | 100.0 | 85 | 100 | −0.562 | −0.601 | 0.039 | |
| | 19 | 50.0 | 100.0 | 20 | 100 | −0.595 | −0.716 | 0.120 | DENV-specific |
| | 20 | 90.0 | 96.7 | 70 | 100 | −0.600 | −0.665 | 0.065 | |
| | 21 | 93.3 | 96.7 | 95 | 95 | −0.014 | −0.016 | 0.002 | Common |
| | 22 | 90.0 | 96.7 | 90 | 90 | −0.427 | −0.404 | 0.022 | |
| | 23 | 90.0 | 96.7 | 90 | 90 | −0.445 | −0.396 | 0.049 | |
| | 24 | 90.0 | 93.3 | 85 | 90 | 0.134 | −0.055 | 0.189 | ZIKV-specific |
| | 25 | 83.3 | 96.7 | 80 | 95 | −0.548 | −0.533 | 0.015 | |

^{†}Patient samples are positive if their normalised peptide responses (calculated as OD of patient sample/mean OD of pooled healthy) are more than 1.01.
^{‡}Binding capacity of a patient positive for a peptide-pair was calculated as: normalised values of [(ZIKV peptide response-DENV peptide response)/DENV peptide response]. Values close to 0 denote equal binding of patient to ZIKV and DENV peptide. Values more than 0 denote a patients preference to bind to ZIKV peptide more than DENV peptide. Values less than 0 denote a patients preference to bind to DENV peptide more than ZIKV peptide.
^{§}Relative difference is calculated as the difference in the mean binding capacity of ZIKV patients and DENV patients. Values are rounded up to 3 decimal places.
^{¶}Common flavivirus epitopes: 60% of ZIKV and DENV patients recognise both ZIKV and DENV peptides of peptide-pair; ZIKV-specific epitopes: 60% of ZIKV patients recognise at least ZIKV peptide of peptide-pair; DENV-specific epitopes: 60% of DENV patients recognise at least DENV peptide of peptide-pair.

Thereafter, new peptides selectively designed based on exposed residues and computational predictions were re-synthesised for subsequent experiments (Table 2).

TABLE 2
ZIKV and DENV peptides information

| | | Amino | | | | | |
|---|---|---|---|---|---|---|---|
| | | acid | | | | | |
| | | position | | | | Peptide | |
| | | on ZIKV | | | | similarity % | |
| | | (accession | | Corresponding | Corresponding | identity, % | Classification |
| | Peptide | KJ776791) | | ZIKV | DENV | query | of potential |
| Protein | no | Start | End | sequence | sequence | cover) | epitope |
| prM | 1 | 6 | 23 | RGSAYYMYLDRNDAGEAI | RDGEPRMIVGKNERGKSL | No | |
| | | | | | | similarity | |
| | 2 | 15 | 32 | DRNDAGEAISFPTTLGMN | GKNERGKSLLFKTASGIN | No | |
| | | | | | | similarity | |
| | 3 | 24 | 41 | SFPTTLGMNKCYIQIMDL | LFKTASGINMCTLIAMDL | No | ZIKV-specific |
| | | | | | | similarity | |
| | 4 | 33 | 50 | KCYIQIMDLGHMCDATMS | MCTLIAMDLGEMCDDTVT | 80%, 55% | |
| | 5 | 42 | 59 | GHMCDATMSYECPMLDEG | GEMCDDTVTYKCPHITE | 62%, 72% | |
| | 6 | 51 | 68 | YECPMLDEGVEPDDVDCW | YKCPHITEVEPEDIDCW | 61%, 100% | |
| | 7 | 56 | 72 | LDEGVEPDDVDCWCNTT | HITEVEPEDIDCWCNLT | 82%, 64% | Common |
| | | | | | | | flavivirus |
| | 8 | 69 | 86 | CNTTSTWVVYGTCHHKKG | CNLTSTWVTYGTCNQAG | 73%, 83% | |
| | 9 | 78 | 92 | YGTCHHKKGEARRSR | TSTWVTYGTCNQAG | 67%, 40% | DENV- |
| | | | | | | | specific |
| | 10 | 100 | 118 | HSTRKLQTRSQTWLESREY | VGMGLDTRTQTVVMSAEGAW | 67%, 47% | |
| E | 11 | 37 | 54 | DKPTVDIELVTTTVSNMA | NKPTLDIELQKTEATQLA | 78%, 50% | |
| | 12 | 61 | 72 | YEASISDMASDS | IEGKITNITTDS | No similarity | |
| | 13 | 73 | 90 | RCPTQGEAYLDKQSDTQY | RCPTQGEAVLPEEQDQNY | 100%, 44% | |
| | 14 | 91 | 108 | VCKRTLVDRGWGNGCGLF | VCKHTYVDRGWGNGCGLF | 89%, 100% | |
| | 15 | 113 | 130 | LVTCAKFACSKKMTGKSI | LVTCAKFQCLEPIEGKVV | 89%, 50% | |
| | 16 | 123 | 140 | KKMTGKSIQPENLEYRIM | EPIEGKVVQYENLKYTVI | 64%, 61% | |
| | 17 | 131 | 149 | PENLEYRIMLSVHGSQHS | YENLKYTVIITVHTGDQH | 53%, 88% | DENV- |
| | | | | | | | specific |
| | 18 | 149 | 166 | SGMIVNDTGHETDENRAK | GDQHQVGNETQGVTAEIT | No similarity | |
| | 19 | 157 | 174 | GHETDENRAKVEITPNSP | GNETQGVTAEITPQASTT | 100%, 22% | |
| | 20 | 166 | 183 | KVEITPNSPRAEATLGGF | TAEITPQASTTEAILPEY | 54%, 72% | |
| | 21 | 191 | 203 | EPRTGLDFSDLYY | SPRTGLDFNEMIL | 70%, 76% | |
| | 22 | 199 | 216 | SDLYYLTMNNKHWLVHKE | NEMILLTMKNKAVVMVHRQ | 62%, 72% | |
| | 23 | 217 | 234 | WFHDIPLPWHAGADTGTP | WFFDLPLPWTSGATTETP | 67%, 100% | |
| | 24 | 235 | 245 | HWNNKEALVEF | TWNRKELLVTF | 70%, 90% | |
| | 25 | 244 | 261 | EFKDAHAKRQTVVVLGSQ | TFKNAHAKKQEVVVLGSQ | 82%, 94% | |
| | 26 | 271 | 288 | GALEAEMDGAKGRLSSGH | GATEIQNSGGTSIFAGH | 100%, 11% | ZIKV-specific |
| | 27 | 306 | 319 | SLCTAAFTFTKIPA | AMCTNTFVLKKEVS | No similarity | |
| | 28 | 325 | 342 | TVTVEVQYAGTDGPCKVP | TILIKVEYKGEDAPCKIP | 62%, 72% | |
| | 29 | 343 | 355 | AQMAVDMQTLTPV | FSTEDGQGKAHN | No similarity | |
| | 30 | 361 | 378 | ANPVITESTENSKMMLEL | ANPVVTKKEEPVNIEA | 83%, 33% | |
| | 31 | 402 | 419 | RSGSTIGKAFEATVRGAK | KKGSSIGKMFEATARGAR | 80%, 83% | |
| | 32 | 453 | 470 | FKSLFGGMSWFSQILIGT | YTALFSGVSWVMKIGIGV | 71%, 38% | ZIKV- |
| | | | | | | | specific |
| NS1 | 33 | 1 | 18 | DVGCSVDFSKKETRCGTG | DMGCVINWKGKELKCGSG | 44%, | |
| | | | | | | 100% | |
| | 34 | 19 | 36 | VFVYNDVEAWRDRYKYHP | IFVTNEVHTVVTEQYKFQA | 41%, 94% | |
| | 35 | 55 | 72 | CGISSVSRMENIMWRSVE | CGIRSTTRMENLLWKQIA | 64%, 77% | |
| | 36 | 70 | 85 | SVEGELNAILEENGVQ | QIANELNYILWENNIK | 70%, 56% | Common |
| | | | | | | | flavivirus |
| | 37 | 91 | 112 | GSVKNPMWRGPQRLPVPVNELP | GDIIGVLEQGKRTLTPQPMELK | No | |
| | | | | | | similarity | |
| | 38 | 119 | 136 | GKSYFVRAAKTNNSFVVD | GKAKIVTAETQNSSFIID | 57%, 38% | Common |
| | | | | | | | flavivirus |
| | 39 | 137 | 154 | GDTLKECPLKHRAWNSFL | GPNTPECPSASRAWNVWE | 70%, 55% | Common |
| | | | | | | | flavivirus |
| | 40 | 155 | 176 | VEDHGFGVFHTSVWLKVREDYS | VEDYGFGVFTTNIWLKLREVYT | 71%, 95% | |
| | 41 | 239 | 256 | SDLIIPKSLAGPLSHHNT | SDMIIPKSLAGPISQHNH | 82%, 94% | |
| | 42 | 248 | 265 | AGPLSHHNTREGYRTQMK | AGPISQHNHRPGYHTQTA | 69%, 88% | |
| | 43 | 257 | 274 | REGYRTQMKGPWHSEELE | RPGYHTQTAGPWHLGKLE | 69%, 72% | DENV- |
| | | | | | | | specific |
| | 44 | 270 | 281 | SEELEIRFEECP | LGKLELDFNYCE | No | |
| | | | | | | similarity | |
| | 45 | 275 | 292 | IRFEECPGTKVHVEETCG | LDFNYCEGTTVVITENCG | 54%, 72% | DENV- |
| | | | | | | | specific |
| | 46 | 284 | 301 | KVHVEETCGTRGPSLRST | TVVITENCGTRGPSLRTT | 85%, 72% | Common |
| | | | | | | | flavivirus |
| | 47 | 293 | 310 | TRGPSLRSTTASGRVIEE | TRGPSLRTTTVSGKLIHE | 72%, | |
| | | | | | | 100% | |
| | 48 | 302 | 319 | TASGRVIEEWCCRECTMP | TVSGKLIHEWCCRSCTLP | 67%, | |
| | | | | | | 100% | |
| | 49 | 315 | 326 | ECTMPPLSFRAK | SCTLPPLRYMGE | 83%, 50% | Common |
| | | | | | | | flavivirus |
| | 50 | 320 | 337 | PLSFRAKDGCVVYGMEIRP | PLRYMGEDGCVVYGMEIRP | 72%, | |
| | | | | | | 100% | |
| | 51 | 338 | 353 | RKEPESNLVRSMVTAG | ISEKEENMVKSLVSAG | No | |
| | | | | | | similarity | |

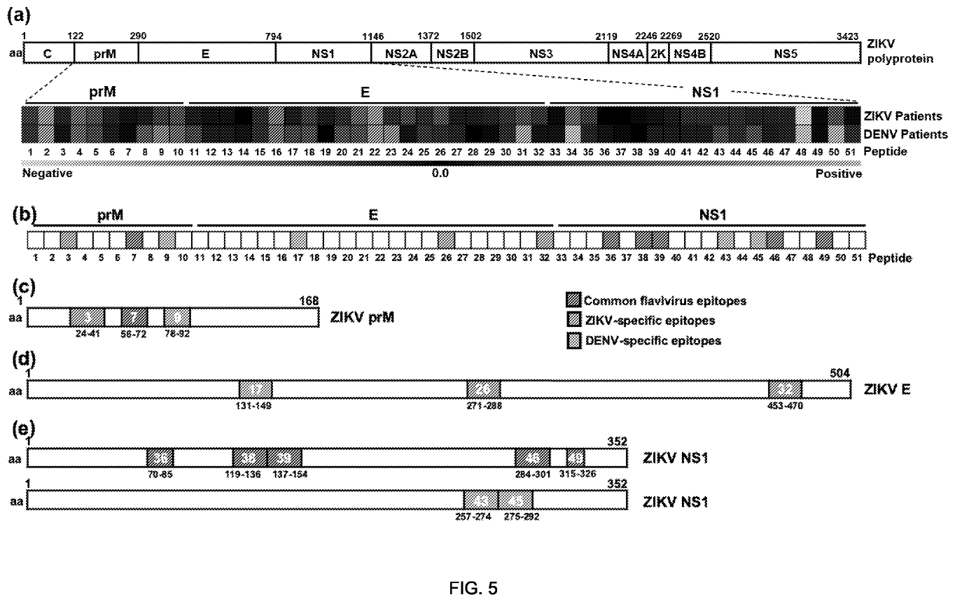

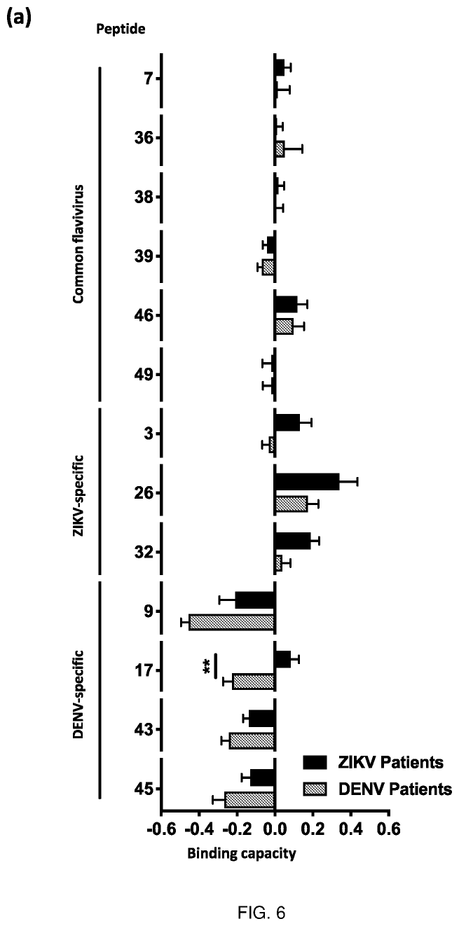

Interestingly, results showed differences between pooled and individual peptides (Table 3, FIG. 5).

TABLE 3
Singapore ZIKV and DENV patients' response to ZIKV and DENV peptides

| | | Percentage recognition (%)^{†} | | | | | | | |
|---|---|---|---|---|---|---|---|---|---|
| | | ZIKV patients | | DENV patients | | Mean binding | | | |
| | | (n = 30-44) | | (n = 20) | | capacity^{‡} | | | |
| | Peptide | ZIKV | DENV | ZIKV | DENV | ZIKV | DENV | Relative | Epitope |
| Protein | no | peptide | peptide | peptide | peptide | patients | patients | difference^{§} | classification^{¶} |
| prM | 1 | 55 | 39 | 55 | 40 | 0.244 | 0.226 | 0.018 | |
| | 2 | 63 | 60 | 95 | 80 | 0.634 | 0.612 | 0.022 | |
| | 3 | 70 | 66 | 80 | 75 | 0.132 | −0.033 | 0.165 | ZIKV-specific |
| | 4 | 30 | 50 | 0 | 15 | −0.356 | −0.346 | 0.010 | |
| | 5 | 59 | 45 | 55 | 50 | 0.306 | 0.272 | 0.034 | |
| | 6 | 59 | 61 | 40 | 50 | −0.120 | −0.079 | 0.041 | |
| | 7 | 86 | 86 | 90 | 85 | 0.056 | 0.014 | 0.042 | Common |
| | 8 | 59 | 64 | 30 | 0 | −0.088 | −0.293 | 0.205 | |
| | 9 | 52 | 55 | 25 | 60 | −0.210 | −0.455 | 0.246 | DENV-specific |
| | 10 | 62 | 86 | 40 | 70 | −0.263 | −0.355 | 0.092 | |
| E | 11 | 64 | 61 | 55 | 35 | 0.066 | 0.271 | 0.205 | |
| | 12 | 84 | 89 | 65 | 65 | 0.065 | 0.076 | 0.011 | |
| | 13 | 59 | 57 | 30 | 20 | −0.030 | 0.088 | 0.118 | |
| | 14 | 68 | 66 | 60 | 65 | −0.015 | 0.043 | 0.059 | |
| | 15 | 61 | 64 | 30 | 50 | −0.138 | −0.157 | 0.020 | |
| | 16 | 53 | 90 | 55 | 85 | −0.415 | −0.379 | 0.036 | |
| | 17 | 70 | 75 | 55 | 85 | 0.084 | −0.227 | 0.311 | DENV-specific |
| | 18 | 70 | 100 | 85 | 100 | −0.218 | −0.260 | 0.042 | |
| | 19 | 57 | 57 | 50 | 55 | 0.084 | 0.006 | 0.078 | |
| | 20 | 60 | 50 | 30 | 20 | 0.186 | 0.314 | 0.129 | |
| | 21 | 54 | 78 | 25 | 45 | −0.314 | −0.264 | 0.050 | |
| | 22 | 47 | 37 | 25 | 0 | 0.610 | 0.620 | 0.010 | |
| | 23 | 34 | 30 | 0 | 0 | 0.152 | N.A. | N.A. | |
| | 24 | 86 | 98 | 90 | 100 | −0.235 | −0.041 | 0.194 | |
| | 25 | 77 | 75 | 55 | 40 | 0.177 | 0.209 | 0.032 | |
| | 26 | 64 | 59 | 25 | 25 | 0.340 | 0.173 | 0.167 | ZIKV-specific |
| | 27 | 68 | 78 | 40 | 50 | −0.081 | −0.114 | 0.033 | |
| | 28 | 87 | 90 | 95 | 95 | 0.095 | −0.001 | 0.097 | |
| | 29 | 76 | 70 | 40 | 45 | −0.109 | −0.018 | 0.090 | |
| | 30 | 64 | 80 | 45 | 55 | −0.182 | −0.157 | 0.025 | |
| | 31 | 93 | 95 | 90 | 75 | 0.319 | 0.681 | 0.362 | |
| | 32 | 100 | 100 | 100 | 100 | 0.189 | 0.039 | 0.150 | ZIKV-specific |
| NSI | 33 | 82 | 86 | 65 | 60 | 0.013 | 0.033 | 0.020 | |
| | 34 | 86 | 82 | 90 | 50 | 0.431 | 0.853 | 0.422 | |
| | 35 | 84 | 89 | 65 | 70 | −0.223 | −0.134 | 0.089 | |
| | 36 | 79 | 83 | 80 | 90 | 0.012 | −0.031 | 0.042 | Common |
| | 37 | 84 | 82 | 60 | 60 | −0.012 | −0.023 | 0.011 | |
| | 38 | 82 | 89 | 60 | 70 | 0.019 | 0.000 | 0.019 | Common |
| | 39 | 89 | 91 | 75 | 75 | −0.041 | −0.069 | 0.027 | Common |
| | 40 | 89 | 91 | 80 | 75 | −0.082 | −0.079 | 0.002 | |
| | 41 | 68 | 66 | 35 | 35 | 0.154 | 0.103 | 0.051 | |
| | 42 | 68 | 59 | 35 | 35 | 0.177 | 0.110 | 0.068 | |
| | 43 | 84 | 91 | 75 | 95 | −0.137 | −0.244 | 0.107 | DENV-specific |
| | 44 | 81 | 83 | 65 | 45 | 0.192 | 0.183 | 0.010 | |
| | 45 | 84 | 89 | 55 | 75 | −0.131 | −0.267 | 0.136 | DENV-specific |
| | 46 | 84 | 82 | 70 | 60 | 0.118 | 0.098 | 0.020 | Common |
| | 47 | 82 | 86 | 50 | 70 | −0.132 | −0.120 | 0.012 | |
| | 48 | 84 | 59 | 65 | 35 | −0.757 | 1.234 | 1.992 | |
| | 49 | 78 | 86 | 50 | 60 | −0.019 | −0.019 | 0.001 | Common |
| | 50 | 80 | 75 | 80 | 40 | 0.334 | 0.720 | 0.386 | |
| | 51 | 86 | 83 | 90 | 80 | 0.019 | 0.160 | 0.141 | |

^{†}Patient samples are positive if their normalised peptide responses (calculated as OD of patient sample/mean OD of pooled healthy) are more than 1.01.
^{‡}Binding capacity of a patient positive for a peptide-pair was calculated using normalised values of: [(ZIKV peptide response-DENV peptide response)/DENV peptide response]. Values close to 0 denote equal recognition of sample to ZIKV and DENV peptide. Values more than 0 denote a sample recognising ZIKV peptide more. Values less than 0 denote a sample recognising DENV peptide more.
^{§}Relative difference is calculated as the difference in the mean binding capacity of ZIKV patients and DENV patients. Values are rounded up to 3 decimal places.
^{¶}Common flavivirus epitopes: 60% of ZIKV and DENV patients recognise both ZIKV and DENV peptides of peptide-pair; ZIKV-specific epitopes: 60% of ZIKV patients recognise at least ZIKV peptide of peptide-pair; DENV-specific epitopes: 60% of DENV patients recognise at least DENV peptide of peptide-pair.

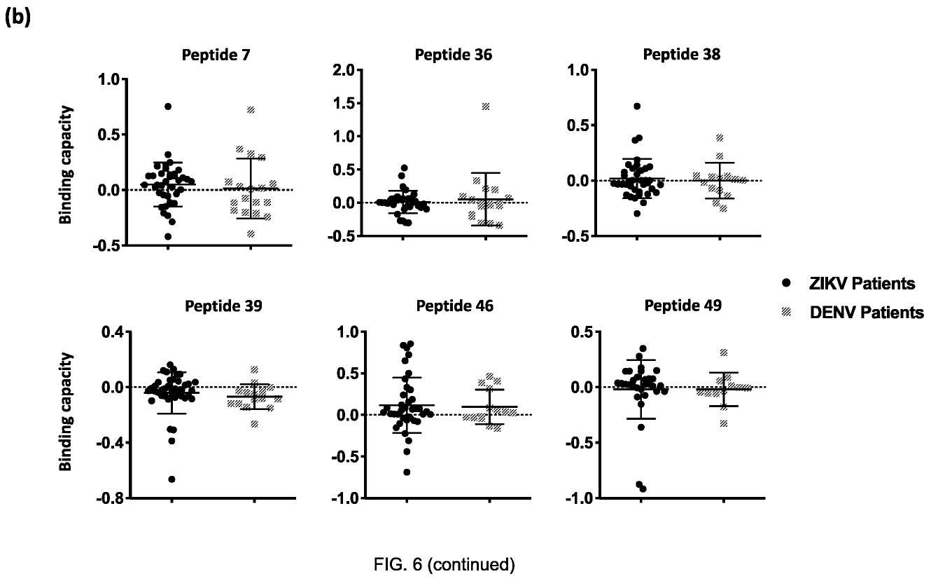

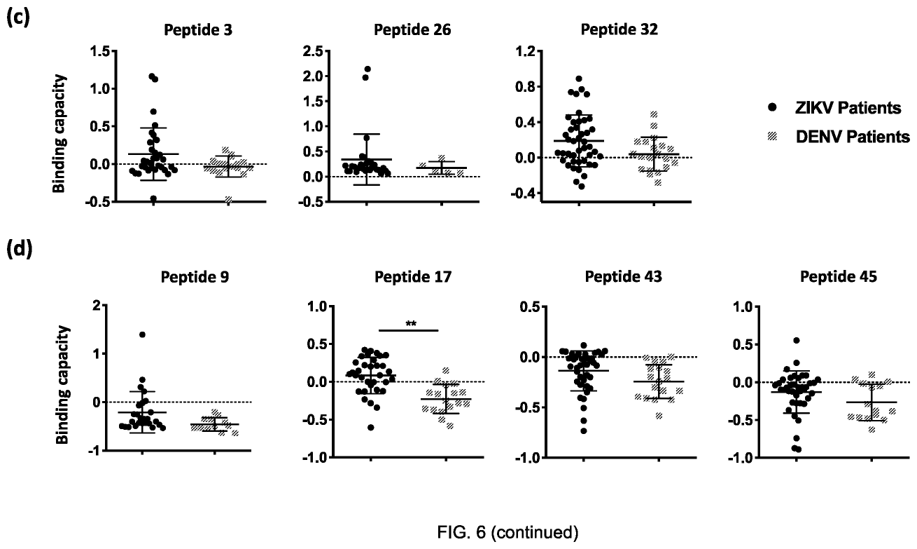

These differences could be due to interferences of the pooled peptides, while single peptides allowed for more enhanced specific binding. Nevertheless, six potential common flavivirus peptides were identified which displayed less than 0.05 relative difference in the binding capacity between ZIKV and DENV patients (peptides 7, 36, 38, 39, 46, 49) (Table 3, FIG. 5, FIG. 6). These peptides were also selected based on the close similarity between the ZIKV and DENV peptide sequence (Table 2). Additionally, three potential ZIKV-specific (peptides 3, 26 and 32), and four potential DENV-specific peptides (peptides 9, 17, 43 and 45) with a binding capacity difference of more than 0.1 were identified (Table 3, FIG. 5, FIG. 6).

### Epitope Recognition by ZIKV Patients Over Time

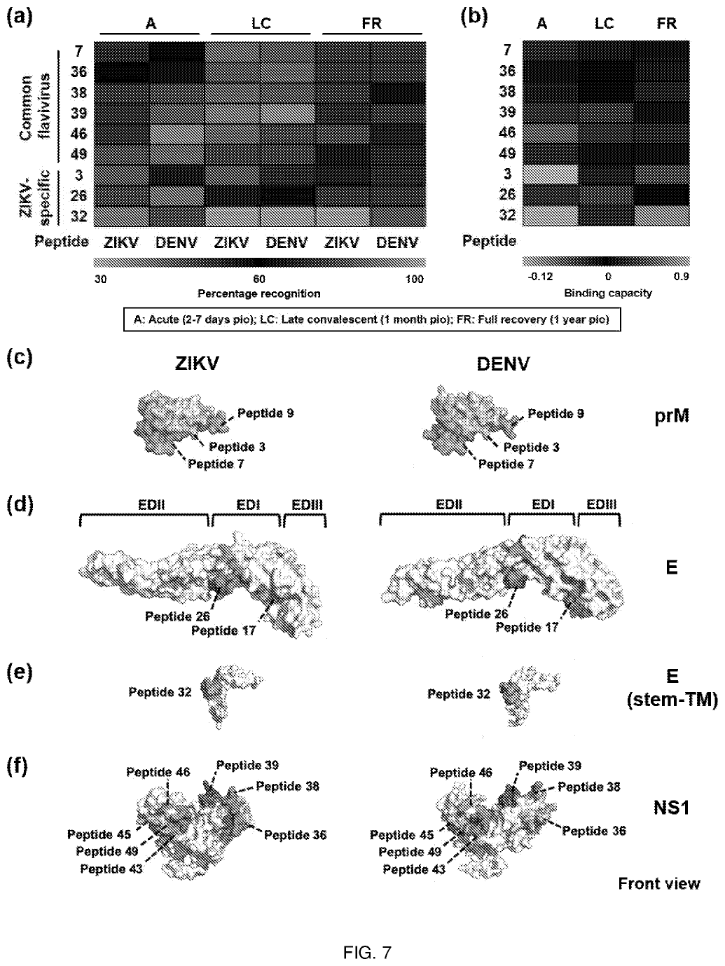

In order to characterise the changes in epitope recognition by ZIKV patients over time, the common flavivirus and ZIKV-specific peptides were screened with plasma of ZIKV patients in acute, late convalescent, and full recovery phases. For the common flavivirus hits, more than 60% of the ZIKV patients were able to recognise the six peptide-pairs at late convalescent and beyond (FIG. 7a). However, at the acute phase, only peptides 7, 36 and 38 were recognised by ZIKV patients (FIG. 7a). In terms of binding capacity, there was equal binding between ZIKV and DENV peptide-pairs over time for peptides 7, 36, 38 and 49 (FIG. 7b).

For ZIKV-specific epitopes, more than 60% of the ZIKV patient samples were able to recognise peptides 3 and 26 (FIG. 7a), with positive peptide binding capacity (FIG. 7b) at late convalescent phase. On the other hand, peptide 32 showed strong recognition by the patient samples (FIG. 7a) as well as high binding capacity (FIG. 7b) at various time points from acute to full recovery. The localisation of all potential epitopes within the viral proteins are shown in FIGS. 7c-f.

Evaluation of Epitopes with Patient Cohorts

To assess the diagnostic performance of identified epitopes, the 13 peptides were screened using patient serum samples from a Thailand cohort that had DENV, bacteria, or unknown infections. Results of a randomised selection of Singapore ZIKV and DENV patients were also analysed in parallel (Table 4).

TABLE 4
Evaluation of patients of different diagnoses and cohorts with potential
linear B-cell epitopes

| | | Peptide | | | | | | | | | | | | |
|---|---|---|---|---|---|---|---|---|---|---|---|---|---|---|
| | | Common flavivirus^{†} | | | | | | ZIKV-specific^{‡} | | | DENV-specific^{‡} | | | |
| Cohort | Patient | 7 | 36 | 38 | 39 | 46 | 49 | 3 | 26 | 32 | 9 | 17 | 43 | 45 |
| ZIKV | 1 | y | y | y | y | y | y | e | z | e | d | z | e | e |
| (Singapore) | 2 | y | y | y | y | n | n | z | n | z | d | d | d | d |
| | 3 | y | y | y | y | y | n | z | z | d | n | z | d | d |
| | 4 | y | y | y | y | y | y | z | z | z | n | z | e | d |
| | 5 | y | y | y | y | y | y | e | z | e | d | z | d | d |
| | 6 | y | y | y | y | y | y | z | z | z | n | e | e | d |
| | 7 | y | y | y | y | y | y | z | z | z | n | e | e | e |
| | 8 | y | n | y | n | y | y | n | n | z | n | d | d | n |
| | 9 | y | y | y | y | y | y | z | z | e | e | z | d | z |
| | 10 | y | y | y | y | y | y | e | z | e | d | z | d | e |
| DENV | 1 | y | y | y | y | y | y | e | e | z | d | z | e | z |
| (Singapore) | 2 | y | n | n | n | n | n | d | n | z | n | n | n | n |
| | 3 | y | y | y | n | n | n | z | n | z | d | d | d | d |
| | 4 | y | y | y | y | y | y | e | n | e | d | d | d | d |
| | 5 | y | y | y | y | y | y | e | z | e | d | d | d | d |
| | 6 | y | n | n | n | n | n | e | n | z | n | n | d | d |
| | 7 | y | y | y | n | y | y | e | n | d | d | e | d | n |
| | 8 | y | n | y | y | y | n | z | n | d | n | d | e | n |
| | 9 | y | y | y | y | y | y | e | z | d | d | d | e | e |
| | 10 | y | y | y | y | y | y | d | e | d | d | e | d | e |
| DENV | 1 | n | y | n | n | n | n | n | n | d | n | n | d | n |
| (Thailand) | 2 | y | y | n | y | n | n | z | n | d | z | d | d | z |
| | 3 | y | n | n | y | n | n | z | n | d | d | d | d | n |
| | 4 | n | n | n | n | n | n | n | n | e | z | d | d | n |
| | 5 | n | n | n | n | n | n | e | n | e | z | d | d | n |
| Bacteria | 1 | y | n | n | y | n | n | d | n | d | z | d | d | n |
| (Thailand) | 2 | n | n | n | n | n | n | d | n | d | z | d | d | n |
| | 3 | y | n | n | n | n | n | e | n | d | z | d | d | n |
| | 4 | y | y | y | y | y | y | e | n | d | z | d | d | e |
| | 5 | y | n | n | n | n | n | d | n | d | z | d | d | n |
| Unknown | 1 | y | y | y | y | y | n | d | z | d | d | d | z | d |
| (Thailand) | 2 | n | n | n | n | n | n | n | n | z | n | d | d | n |
| | 3 | y | y | y | y | y | n | d | n | d | d | d | d | n |
| | 4 | y | n | y | y | n | n | d | n | d | z | d | d | n |
| | 5 | y | n | y | y | n | n | n | n | d | n | d | d | n |
| | 6 | y | y | y | y | y | y | e | z | d | d | d | d | e |
| | 7 | n | n | n | n | n | n | n | n | d | n | n | n | n |
| | 8 | n | n | n | n | y | n | d | n | d | n | n | d | n |

^{†}A patient sample is considered positive (indicated as “y”) if it has a normalised peptide response higher than pooled healthy control for both ZIKV and DENV peptide-pair. If a sample peptide-pair response is lower than the healthy, it is considered negative (indicated as “n”)
^{‡}If a patient sample is positive for a peptide, i.e. has a higher normalised peptide response than pooled healthy control, the binding capacity of peptides (calculated as [(ZIKV peptide response-DENV peptide response)/DENV peptide response] was then determined. For patients with peptide binding capacity values of i) binding capacity ≥ 0.1 → positive for ZIKV peptide (indicated as “z”) ii) binding capacity ≤ 0.1 → positive for DENV peptide (indicated as “d”) ii) −0.1 < binding capacity < 0.1 → equal recognition of ZIKV and DENV peptide-pair (indicated as “e”)

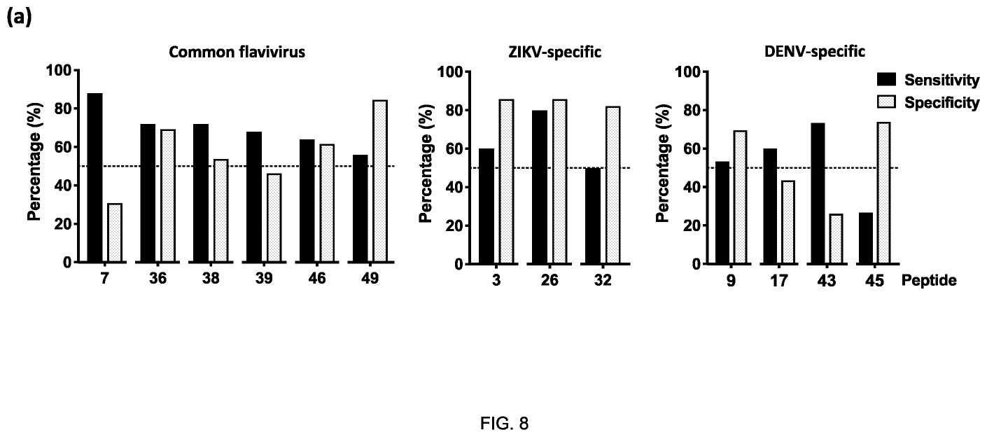

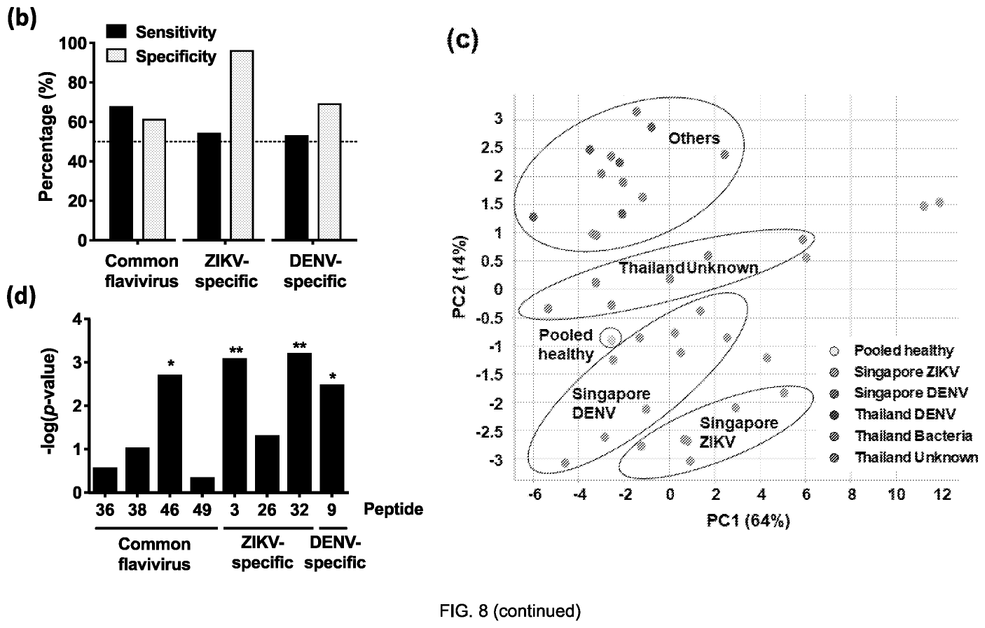

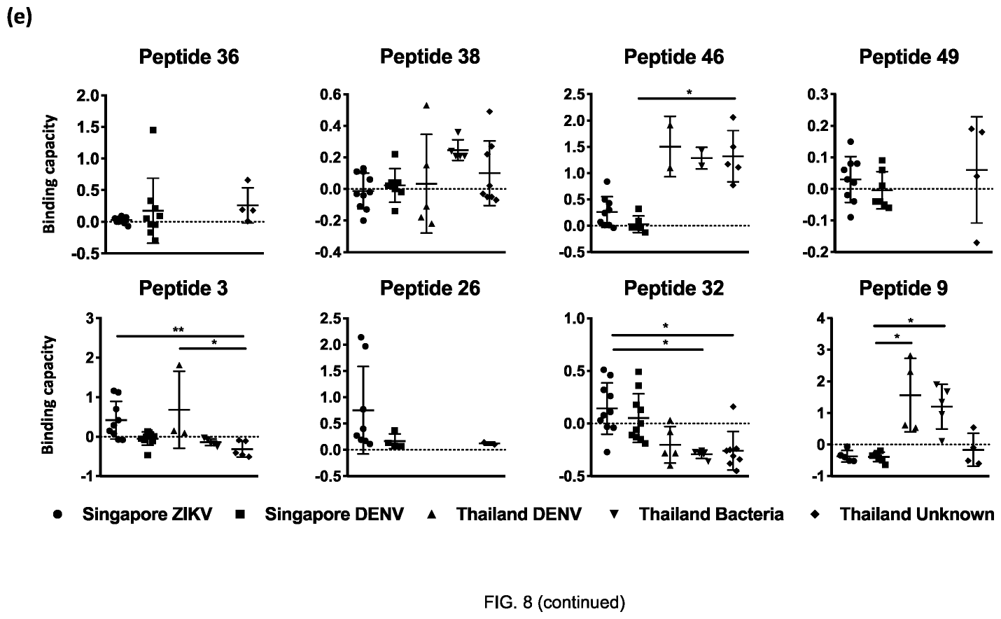

Interestingly, results showed a wide range of specificity and sensitivity for each peptide (Table 5, FIG. 8a).

TABLE 5
Diagnostic evaluation of linear B-cell epitopes

| | | | | No of patients^{†} | | | | | |
|---|---|---|---|---|---|---|---|---|---|
| | Epitope | | Peptide | True | True | False | False | Sensitivity | Specificity |
| Analysis | classification | Protein | no | positive | negative | negative | positive | (%)^{‡} | (%)^{¶} |
| Individual | Common | prM | 7 | 22 | 4 | 3 | 9 | 88.0 | 30.8 |
| peptide | flavivirus | NS1 | 36 | 18 | 9 | 7 | 4 | 72.0 | 69.2 |
| | | | 38 | 18 | 7 | 7 | 6 | 72.0 | 53.8 |
| | | | 39 | 17 | 6 | 8 | 7 | 68.0 | 46.2 |
| | | | 46 | 16 | 8 | 9 | 5 | 64.0 | 61.5 |
| | | | 49 | 14 | 11 | 11 | 2 | 56.0 | 84.6 |
| | ZIKV- | prM | 3 | 6 | 24 | 4 | 4 | 60.0 | 85.7 |
| | specific | E | 26 | 8 | 24 | 2 | 4 | 80.0 | 85.7 |
| | | | 32 | 5 | 23 | 5 | 5 | 50.0 | 82.1 |
| | DENV- | prM | 9 | 8 | 16 | 7 | 7 | 53.3 | 69.6 |
| | specific | E | 17 | 9 | 10 | 6 | 13 | 60.0 | 43.5 |
| | | NS1 | 43 | 11 | 6 | 4 | 17 | 73.3 | 26.1 |
| | | | 45 | 4 | 17 | 11 | 6 | 26.7 | 73.9 |
| Peptide | Common | NS1 | 36 | 17 | 8 | 8 | 5 | 68 | 61.5 |
| combination | flavivirus | | 38 | | | | | | |
| | | | 46 | | | | | | |
| | | | 49 | | | | | | |
| | ZIKV- | prM | 3 | 6 | 27 | 5 | 1 | 54.5 | 96.4 |
| | specific | E | 26 | | | | | | |
| | | | 32 | | | | | | |
| | DENV- | prM | 9 | 8 | 16 | 7 | 7 | 53.3 | 69.6 |
| | specific | | | | | | | | |

^{†}ZIKV (n = 10) and DENV (n = 10) patients from Singapore, and DENV (n = 5), bacteria (n = 5) and unknown (n = 8) patients from Thailand were used in the diagnostic evaluation.
^{‡}Sensitivity is calculated as the percentage of [true positive patients/(true positive patients + false negative patients)].
^{¶}Specificity is calculated as the percentage of [true negative patients/(true negative patients + false positive patients)].

ZIKV-specific peptide 26 (amino acid residues 271-288) on the E protein of domain I/II (EDI/II) had the best sensitivity and specificity profile (80% and 85.7% respectively) (Table 5, FIG. 8a). Nevertheless, eight peptides (common flavivirus peptides 36, 38, 46, 49; ZIKV-specific peptides 3, 26, 32; and DENV-specific peptide 9) showed more than 50% sensitivity and specificity (Table 5, FIG. 8a), and were selected for further evaluation. These peptides were used to “diagnose” the patients (Table 6), and the performance of the peptide combination based on the epitope groupings were determined collectively (Table 5, FIG. 8b).

TABLE 6
Evaluation of common and differential flavivirus peptide mix with patient cohorts

| | | Percentage of positive peptides (%) | | | Outcome of patients | | | |
|---|---|---|---|---|---|---|---|---|
| | | Common | ZIKV- | DENV- | Common | ZIKV- | DENV- | Final |
| Cohort | Patient | flavivirus^{†} | specific^{‡} | specific^{§} | flavivirus^{†} | specific^{‡} | specific^{§} | “diagnosis”^{¶} |
| ZIKV | 1 | 100 | 33.3 | 100 | y | n | y | d |
| (Singapore) | 2 | 50 | 66.7 | 100 | n | y | y | n |
| | 3 | 100 | 66.7 | 0 | y | y | n | z |
| | 4 | 100 | 100.0 | 0 | y | y | n | z |
| | 5 | 100 | 33.3 | 100 | y | n | y | d |
| | 6 | 100 | 100.0 | 0 | y | y | n | z |
| | 7 | 100 | 100.0 | 0 | y | y | n | z |
| | 8 | 75 | 33.3 | 0 | y | n | n | n |
| | 9 | 100 | 66.7 | 0 | y | y | n | z |
| | 10 | 100 | 33.3 | 100 | y | n | y | d |
| DENV | 1 | 100 | 33.3 | 100 | y | n | y | d |
| (Singapore) | 2 | 0 | 33.3 | 0 | n | n | n | n |
| | 3 | 50 | 66.7 | 100 | n | y | y | n |
| | 4 | 100 | 0.0 | 100 | y | n | y | d |
| | 5 | 100 | 33.3 | 100 | y | n | y | d |
| | 6 | 25 | 33.3 | 0 | n | n | n | n |
| | 7 | 75 | 0.0 | 100 | y | n | y | d |
| | 8 | 75 | 33.3 | 0 | y | n | n | n |
| | 9 | 100 | 33.3 | 100 | y | n | y | d |
| | 10 | 100 | 0.0 | 100 | y | n | y | d |
| DENV | 1 | 25 | 0.0 | 0 | n | n | n | n |
| (Thailand) | 2 | 75 | 33.3 | 0 | y | n | n | n |
| | 3 | 50 | 33.3 | 100 | n | n | y | n |
| | 4 | 25 | 0.0 | 0 | n | n | n | n |
| | 5 | 25 | 0.0 | 0 | n | n | n | n |
| Bacteria | 1 | 50 | 0.0 | 0 | n | n | n | n |
| (Thailand) | 2 | 25 | 0.0 | 0 | n | n | n | n |
| | 3 | 25 | 0.0 | 0 | n | n | n | n |
| | 4 | 100 | 0.0 | 0 | y | n | n | n |
| | 5 | 25 | 0.0 | 0 | n | n | n | n |
| Unknown | 1 | 100 | 33.3 | 100 | y | n | y | d |
| (Thailand) | 2 | 25 | 33.3 | 0 | n | n | n | n |
| | 3 | 100 | 0.0 | 100 | y | n | y | d |
| | 4 | 75 | 0.0 | 0 | y | n | n | n |
| | 5 | 50 | 0.0 | 0 | n | n | n | n |
| | 6 | 100 | 33.3 | 100 | y | n | y | d |
| | 7 | 25 | 0.0 | 0 | n | n | n | n |
| | 8 | 50 | 0.0 | 0 | n | n | n | n |

Based on Table 4, the percentage of positive peptides were calculated and “outcome” of patients were
assigned.
^{†}If sample is positive for 3 or more (out of 4) common flavivirus peptides, i.e. 75% positive, the patient
is considered positive (indicated as “y” in the outcome) for flavivirus infection. If sample is ≥75%
positive, the patient is considered negative (indicated as “n” in the outcome).
^{‡}If sample is positive for 2 or more (out of 3) ZIKV-specific peptides, i.e. ≥66.7% positive, the patient
is considered positive (indicated as “y” in the outcome) for ZIKV infection. If sample is ≤66.7%
positive, the patient is considered negative (indicated as “n” in the outcome).
^{§}If sample is positive for the DENV-specific peptide, i.e. ≥100% positive, the patient is considered
positive (indicated as “y” in the outcome) for DENV infection, whereas negative for the DENV-
specific peptide is indicated as “n” in the outcome.
^{¶}Based on the respective outcomes of the epitope categories, the following combinations produce the
final ZIKV and DENV “diagnosis” of patients:

| | | | |
|---|---|---|---|
| Common flavivirus | ZIKV-specific | DENV-specific | Final “diagnosis” |
| y | y | n | ZIKV positive (“z”) |
| y | n | y | DENV positive (“d”) |
| y | n | n | ZIKV and DENV negative (“n”) |
| n | y | n | |
| n | n | y | |
| n | n | n | |

Although the common flavivirus and DENV-specific groups demonstrated modest measurements, the ZIKV-specific peptide mix showed a robust specificity of 96.4% (Table 5, FIG. 8b). Furthermore, when the anti-peptide IgG response of patients was plotted in a principal component analysis (PCA), it was observed that patients of different diagnoses and cohorts formed separate dusters, and ZIKV patients stood out when compared to the healthy control (FIG. 8c). To identify peptides with discriminating power, the binding capacity of positive peptides were calculated. The virus-specific ZIKV and DENV epitopes were significantly differential (FIG. 8d). Peptide 32 (amino acid residues 453-470 on E protein) was the best performing ZIKV-specific epitope, and was able to distinguish Singapore ZIKV patients from bacteria and unknown infections from Thailand (FIGS. 8d-e). DENV-specific peptide 9 (amino acid residues 78-92 on prM) could be used to differentiate Singapore DENV patients from bacteria-infected patients from Thailand (FIG. 8e). Overall, we have identified the best differential epitopes to differentiate between DENV and ZIKV patients.

### Conclusion

ZIKV patients were shown to produce high levels of ZIKV-specific IgG antibodies. Specifically, IgG1 and IgG3 were the subclasses induced following ZIKV infection, closely resembling DENV-infected patients. Although patients from this cohort had detectable DENV IgG levels due to the high level of cross-reactivity among flaviviruses, DENV neutralisation was significantly less efficient compared to ZIKV, indicating that the antibodies were ZIKV-specific (FIGS. 2e-f, FIG. 3). This observation is also supported by another study, in which the profiles of ZIKV neutralising antibodies of patients from Nicaragua, Sri Lanka and Thailand were not affected by previous DENV infection. Nonetheless, it is imperative to consider the possible implications of virus-infection enhancement. Moreover, none of the ZIKV patients in our study displayed severe symptoms to suggest occurrence of antibody-dependent enhancement (ADE), and similar observations were also reported from Brazil.

Peptides identified from B-cell epitope mapping have been reported on flavivirus E, prM NS1 and NS3 antigens from antibodies of patients and animal models. Identification of antigenic epitopes and characterisation of cross-reactive epitopes are crucial in vaccine and immunodiagnostic developments. While various reports have shown the specificity of the NS1 antigen to differentiate between ZIKV and DENV, majority of the common flavivirus peptides identified in this disclosure are on the NS1 protein, possibly due to the conserved regions of NS1 amongst the flaviviruses. For example, common flavivirus peptides 36 (amino acid residues 70-85), 38 (amino acid residues 119-136) and 49 (amino acid residues 315-326) were identified as ZIKV-specific in other patient cohorts from South America. However, it remains to be seen if these peptides could be used to detect all flaviviruses such as yellow fever virus (YFV) and Japanese encephalitis virus (JEV).

Differential ZIKV and DENV epitopes identified were located across prM, E and NS1. Of interest, DENV-specific peptide 17 (amino acid residues 131-149) and ZIKV-specific peptide 26 are found on EDI and EDII of E glycoprotein, which share 35% and 51% amino acid identity between ZIKV and DENV respectively, whereas ZIKV-specific peptide 32 is found in the transmembrane domain of the anchor region (FIG. 7e). Interestingly, peptide 32 (amino acid residues 453-470) maps to a region that overlaps with a DENV-2 epitope (amino acid residues 451-468) described for immune sera of DENV-2 infected patients. Computational analyses of ZIKV-specific peptide 32 and DENV-2-equivalent epitope showed that they remain moderately accessible on the virus particle. Since they share low sequence identity (43.75%), this epitope could be conformationally different, and thus differentially recognised by ZIKV and DENV-specific antibodies. It would also be useful to assess the use of the identified peptides as a ZIKV vaccine target, particularly peptides 26 and 32. Interestingly, despite the similarity between the sequence of these ZIKV and DENV peptide-pairs (Table 2), they were able to distinguish ZIKV and DENV patients. Moreover, ZIKV patients at different disease stages have different peptide recognition, and the current set-up could identify ZIKV infection at any point, independent of the patients' level of ZIKV-specific antibodies (FIGS. 9b-c). However, given that the identified epitopes were screened and validated using adult patient samples, it would be important to assess how these epitope profiles will perform in other patient cohorts, specifically ZIKV-infected pregnant women from Brazil.

Identified putative epitopes were preliminary diagnostically evaluated with 38 patient samples. Intriguingly, the Singapore DENV and Thailand DENV patients were not clustered together in the PCA (FIG. 8c). Most of the Singapore DENV patients selected for validation had moderate to severe forms of plasma leakage, a clinical feature of severe manifestations of DENV infection, whereas DENV patients from Thailand displayed mild symptoms (unpublished data). The latter being “negative” in our assays could thus be due to differences in epitope recognition in different DENV disease states, and the different strain of viruses circulating in Singapore and Thailand. Nonetheless, further refinements are required to identify serotype-specific DENV epitopes.

Furthermore, comparing these results and computationally-predicted diagnostic peptide regions revealed differences. Firstly, majority of the computationally predicted peptide regions were not ZIKV-specific. NS1 peptide 36, for example, was predicted to be differential, but was in fact a common flavivirus. Furthermore, peptide 26 on E glycoprotein, predicted to recognise both ZIKV and DENV, was shown to be ZIKV-specific in this disclosure. Despite differences in various approaches, computational prediction remains a useful tool.

Overall, this disclosure offers important valuable information on the human antibody response against ZIKV and insights into epitope cross-reactivity. Notably, several novel differential ZIKV and DENV epitopes with potential diagnostic efficacies have been identified on prM and E proteins. These results offer useful insights towards the development of diagnostics or vaccines.

### Methods

### Ethics Statement

Written informed consent was obtained from participants in accordance with the tenets of the Declaration of Helsinki. Study protocols of Singapore ZIKV (2016-2018) and DENV (2010-2012) patient cohorts were approved by the SingHealth Centralised Institutional Review Board (CIRB Ref: 2016/2219) and National Healthcare Group (NHG) Domain Specific Review Board (DSRB-E-2009/432) respectively. Specimens from Singapore healthy donors (2010-2015) and patients from Thailand (2011-2013) were collected in accordance to study guidelines of approval numbers: NUS-IRB 09-256 and NUS-IRB 10-445; MUTM 2011-008-01, OXTREC 42-10 and TCAB-01-11 respectively.

### Study Subjects and Sample Collection

### Singapore ZIKV Patients

Collection of specimens from subjects during the ZIKV outbreak in 2016 was previously described³⁰. Briefly, 65 patients that were RT-PCR positive for ZIKV in whole blood or urine, and negative for DENV RT-PCR were enrolled. Whole blood specimens were collected in EDTA-coated vacutainer tubes (Becton Dickinson, Franklin Lakes, N.J., USA) after peripheral venipuncture and were centrifuged at 12000 rpm for 10 min. Plasma was collected and heat-inactivated for 30 min at 56° C. before storage at −80° C. Specimens were obtained over a period of six time points: (1) acute [2-7 days post-illness onset (pio)], (2) early convalescent (10-14 days pio), (3) late convalescent (1 month pio), (4) early recovery (3 months pio), (5) late recovery (5-6 months pio), and (6) full recovery (1 year pio) phases.

### Singapore DENV Patients

Twenty DENV patient serum samples (2010-2012) collected before the ZIKV outbreak were used in this study. Patients were DENV PCR and/or NS1 positive upon hospital admission, and were a combination of the following: one unknown serotype, six DENV-1, seven DENV-2, three DENV-3, and three DENV-4 patients. Serum samples used were obtained at late convalescent phase (21-37 days pio).

### Thailand Patients

Archived serum samples from an undifferentiated fever study conducted at Shoklo Malaria Research Unit (SMRU) were used. Five DENV patients were confirmed by gold standard paired serology, and all but one was DENV PCR positive. Five bacteria-infected patients were diagnosed with leptospirosis, scrub typhus, murine typhus or Streptococcus pneumoniae infections, or a combination of above, and all were DENV PCR and DENV NS1, IgM and IgG RDT negative. Eight patients with unknown diagnoses were negative for the above pathogens by serology, blood culture and PCR. Convalescent serum samples used were collected at 14-20 days pio.

### Viruses

ZIKV Polynesian isolate (H/PF/2013) was obtained from the European Virus Archive (EVA, Marseille, France). DENV-3 was used as a reference DENV serotype because it is widespread in Southeast Asia, and was kindly provided by the National Public Health Laboratory (NPHL), Singapore. CHIKV SGP011 was isolated from a patient from Singapore. Viruses were propagated in VeroE6 cells (ATCC, Manassas, Va., USA) and purified via ultracentrifugation before being titered by standard plaque assays in VeroE6 cells.

### Virion-Based ELISA

Antibody titres were determined by a virion-based ELISA as previously described^{18, 24-27}. Briefly, purified virus was immobilised on 96-well maxisorp microtitre plates overnight (Thermo Fisher Scientific, Waltham, Mass., USA). Wells were blocked with 0.05% PBST [0.05% Tween-20 (Sigma-Aldrich, Saint Louis, Mo., USA) in PBS] containing 5% skim milk (Nacalai Tesque, Kyoto, Japan) at 37° C. for 1.5 h. Heat-inactivated patient and pooled healthy control plasma samples at 1:200 to 1:8000 dilutions prepared in PBST with 2.5% milk were incubated at 37° C. for 1 h. HRP-conjugated goat anti-human IgM or IgG (H+L) (Thermo Fisher Scientific) or mouse anti-human IgG1, IgG2, IgG3 and IgG4 (Thermo Fischer Scientific) antibodies were used for detection. Reactions were developed using TMB (3,3,5,5-tetramethyl benzidine) substrate (Sigma-Aldrich) and terminated with Stop reagent (Sigma-Aldrich), and absorbance was measured at 450 nm in a microplate autoreader (Tecan, Männedorf, Zürich, Switzerland) 18, 24-27. ELISA readings were conducted in duplicates or triplicates.

### Sero-Neutralisation

Neutralising capacity of antibodies from ZIKV patients were determined via flow cytometry. Briefly, pooled patient and healthy plasma samples at 1:500, 1:1000 and 1:2000 dilutions were incubated with ZIKV or DENV-3 at MOI 10 for 2 h at 37° C. with gentle agitation (350 rpm). Virus-antibody suspensions were then added in duplicates to HEK 293T cells (ATCC) at 37° C. After 2 h, media were removed and Dulbecco's Modified Eagle Medium (DMEM; GE Healthcare Life Sciences, Pittsburgh, Pa., USA) with 10% foetal bovine serum (FBS; GE Healthcare Life Sciences) were added. After 48 h, cells were harvested and stained as described⁵⁴, using ZIKV NS3 protein-specific rabbit polyclonal antibody or DENV human monoclonal antibody 1B25, and counter-stained with fluorophore-tagged goat anti-rabbit or anti-human IgG (H+L) (Thermo Fisher Scientific) respectively. Cells were acquired with MacsQuant Analyser 10 (Miltenyi-Biotec, Bergisch Gladbach, Germany). Assay was carried out in duplicates with two independent experiments. Flow cytometry results were analysed with FlowJo (version 10.4.1, Tree Star Inc, Ashland, Oreg., USA). Data of patient and pooled healthy neutralisation assays were normalised using the respective untreated infections and calculated as a percentage of virus-only control infection.

### Epitopes Determination

### Linear Peptide Libraries

The sequences used for the design of biotinylated linear peptides of prM, E and NS1 proteins were derived from ZIKV Polynesian isolate (KJ776791) and consensus sequence of DENV-3 strains (KR296743, KF973487, EU081181, KF041254, JF808120, JF808121, KJ189293, KC762692, KC425219, KJ830751, KF973479, and AY099336). Peptides were generated as a ZIKV and DENV peptide-pair of corresponding sequences. Preliminary epitope screening was used with a library of peptides (Mimotopes, Mulgrave, Victoria, Australia) consisting of 18-mer overlapping sequences. Five peptides were combined to form one pooled peptide set. Screening and validation of patients were done with higher purity of peptides ((290%, EMC microcollections GmbH, Tuebingen, Germany) with lengths ranging from 11 to 22-mer (Table 2). Peptides were dissolved in DMSO (Sigma-Aldrich) to obtain a stock concentration of 3.75 μg μl⁻¹.

### Peptide-Based ELISA

Epitope determination was performed via peptide-based ELISA as previously described^{24,25,27}. Briefly, streptavidin-coated plates (Thermo Fisher Scientific) were blocked with 0.1% PBST (0.1% Tween-20 in PBS) containing 1% sodium caseinate (Sigma-Aldrich) and 1% bovine serum albumin (BSA; Sigma-Aldrich) overnight at 4° C., before addition of biotinylated peptides (1:1000 dilution in 0.1% PBST), followed by heat-inactivated pooled healthy control and patient plasma/serum samples (1:2000 dilution in 0.1% PBST). HRP-conjugated goat-anti human IgG (H+L) antibody (Thermo Fisher Scientific) prepared in 0.1% blocking buffer was used for detection of peptide-bound antibodies. TMB substrate and Stop reagent (Sigma-Aldrich) were used for development, prior to absorbance measurements at 450 nm (Tecan). All incubation steps were at room temperature for 1 h on a rotating shaker, and ELISA readings were conducted in duplicates.

### Data Analysis

OD values obtained from ZIKV and DENV peptide-based ELISA experiments were first normalised against mean OD values of pooled healthy donors. Patient samples were considered positive if the normalised response was more than 1.01. Subsequently, peptide binding capacity was calculated using the normalised values as [(ZIKV peptide response−DENV peptide response)/DENV peptide response]. Binding capacities with positive values denote the binding preference of the sample to ZIKV peptide, whereas negative values denote a binding preference to the corresponding DENV peptide. Difference in the mean peptide binding capacity of ZIKV patients and DENV patients of a peptide-pair (i.e. ZIKV and DENV peptides with complementary sequence) was calculated. Peptides with a relative difference of 0.1 or more are considered to be differential ZIKV and DENV epitopes of interest, whereas peptides with a difference of 0.05 or less, and share amino acid similarity between the peptide-pair (Table 2) are considered as common flavivirus epitopes.

### Data Visualisation and Statistical Analysis

Heat-maps were generated using Multi Experiment Viewer (version 4.8, Microarray Software Suite TM4, Boston, Mass., USA). For structural localisation, ZIKV prM was simulated using Phyre (version 2, Structural Bioinformatics Group, London, UK) 55. Structures of DENV-3 prM, ZIKV E glycoprotein, DENV-3 E glycoprotein, ZIKV stem-transmembrane domain of E glycoprotein, DENV-3 stem-transmembrane domain of E glycoprotein, ZIKV NS1 and DENV-3 NS1 were modelled based on PDB 3C6E, 5JHM, 1UZG, 51Z7, 3J2P, 5K6K and 406B respectively. All structures were visualised using PyMol (Schrödinger, Cambridge, Mass., USA). Principal component analysis (PCA) was performed using the OD values of the anti-peptide IgG response by patients using prcomp function in R (version 3.3.1; R Foundation for Statistical Computing, Vienna, Austria).

Statistics were done using GraphPad Prism (version 7.03, San Diego, Calif., USA). Mann-Whitney U tests, two-tailed, with Bonferroni correction for multiple testing, or Kruskal-Wallis tests with Bonferroni correction for multiple testing, and post hoc tests using Dunn's multiple comparison tests were used to derive any statistical significance. Correlation analysis was carried out using Spearman's rank correlation. P-values less than 0.05 are considered significant.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

### REFERENCES

1 Teixeira M G, Costa Mda C, de Oliveira W K, Nunes M L, Rodrigues L C. The epidemic of Zika virus-related microcephaly in Brazil: detection, control, etiology, and future scenarios. Am J Public Health 2016; 106: 601-605.
2 Barcellos C, Xavier D R, Pavão A L et al. Increased hospitalizations for neuropathies as indicators of Zika virus infection, according to health information system data, Brazil. Emerg Infect Dis 2016; 22: 1894-1899.
3 He D, Gao D, Lou Y, Zhao S, Ruan S. A comparison study of Zika virus outbreaks in French Polynesia, Colombia and the state of Bahia in Brazil. Sci Rep 2017; 7: 1-6.
4 Cauchemez S, Besnard M, Bompard P et al. Association between Zika virus and microcephaly in French Polynesia, 2013-2015: a retrospective study. Lancet 2016; 387: 2125-2132.
5 Wilson H L, Tran T, Druce J, Dupont-Rouzeyrol M, Catton M. Neutralization assay for Zika and dengue viruses by use of real-time-PCR-based endpoint assessment. J Clin Microbiol 2017; 55: 3104-3112.
6 Goncalves A, Peeling R W, Chu M C et al. Innovative and new approaches to laboratory diagnosis of Zika and dengue: a meeting report. J Infect Dis 2018; 217: 1060-1068.
7 Priyamvada L, Quicke K M, Hudson W H et al. Human antibody responses after dengue virus infection are highly cross-reactive to Zika virus. Proc Natl Acad Sci 2016; 113: 7852-7857.
8 Stettier K, Beltramello M, Espinosa D A et al. Specificity, cross-reactivity and function of antibodies elicited by Zika virus infection. Science 2016; 353: 823-826.
9 Kawiecki A B, Christofferson R C. Zika virus-induced antibody response enhances dengue virus serotype 2 replication in vitro. J Infect Dis 2016; 214:1357-1360.
10 Barba-Spaeth G, Dejnirattisai W, Rouvinski A et al. Structural basis of potent Zika-dengue virus antibody cross-neutralization. Nature 2016; 536: 48-53.
11 Wong S J, Furuya A, Zou J et al. A multiplex microsphere immunoassay for Zika virus diagnosis. EBioMedicine 2017; 16: 136-140.
12 Chang H-H, Huber R G, Bond P J et al. Systematic analysis of protein identity between Zika virus and other arthropod-borne viruses. Bull World Health Organ 2017; 95: 517-5251.
13 Xu X, Vaughan K, Weiskopf D et al. Identifying candidate targets of immune responses in Zika virus based on homology to epitopes in other flavivirus species. PLoS Curr 2016; 8: ecurrents.outbreaks.9aa2e1fb61b0f632f58a09877 3008c4b.
14 Xu X, Song H, Qi J et al. Contribution of intertwined loop to membrane association revealed by Zika virus full-length NS1 structure. EMBO J 2016; 35: 2170-2178.
15 Harrison S C. Immunogenic cross-talk between dengue and Zika viruses. Nat Immunol 2016; 17: 1010-1012.
16 Mohns M S, Bailey A, Breitbach M E et al. Antibody responses to Zika virus proteins in pregnant and non-pregnant macaques. PLoS Negl Trop Dis 2018; 12: e0006903.
17 Funk S, Kucharski A J, Camacho A et al. Comparative analysis of dengue and Zika outbreaks reveals differences by setting and virus. PLoS Negl Trop Dis 2016; 10: 1-16.
18 Chia P Y, Yew H S, Ho H et al. Clinical features of patients with Zika and dengue virus co-infection in Singapore. J Infect 2017; 74: 611-615.
19 Campos G, Bandeira A, Sardi S. Zika virus outbreak, Bahia, Brazil. Emerg Infect Dis 2015; 21: 1885-1886.
20 Steinhagen K, Probst C, Radzimski C et al. Serodiagnosis of Zika virus (ZIKV) infections by a novel NS1-based ELISA devoid of cross-reactivity with dengue virus antibodies: a multicohort study of assay performance, 2015 to 2016. Eurosurveillance 2016; 21: 1-16.
21 Balmaseda A, Stettier K, Medialdea-Carrera R et al. Antibody-based assay discriminates Zika virus infection from other flaviviruses. Proc Natl Acad Sci 2017; 114: 8384-8389.
22 Mishra N, Caciula A, Price A et al. Diagnosis of Zika virus infection by peptide array and ELISA. MBio 2018; 9: 1-16.
23 Lee A J, Bhattacharya R, Scheuermann R H, Pickett B E. Identification of diagnostic peptide regions that distinguish Zika virus from related mosquito-borne Flaviviruses. PLoS One 2017; 12: 1-18.
24 Kam Y W, Lum F M, Teo T H et al. Early neutralizing IgG response to chikungunya virus in infected patients targets a dominant linear epitope on the E2 glycoprotein. EMBO Mol Med 2012; 4: 330-343.
25 Kam Y-W, Lee C Y-P, Teo T-H et al. Cross-reactive dengue human monoclonal antibody prevents severe pathologies and death from Zika virus infections. JCI Insight 2017; 2: 1-10.
26 Kam Y W, Simarmata D, Chow A et al. Early appearance of neutralizing immunoglobulin G3 antibodies is associated with chikungunya virus clearance and long-term clinical protection. J Infect Dis 2012; 205: 1147-1154.
27 Kam Y-W, Leite J A, Amrun S N et al. ZIKV-specific NS1 epitopes as serological markers of acute Zika virus infection. J Infect Dis 2019; e-pub ahead of print 7 Mar. 2019; doi:10.1093/infdis/jiz092.
28 Ho Z J M, Hapuarachchi H C, Barkham T et al. Outbreak of Zika virus infection in Singapore: an epidemiological, entomological, virological, and clinical analysis. Lancet Infect Dis 2017; 17: 813-821.
29 Lum F-M, Lin C, Susova O Y et al. A sensitive method for detecting Zika virus antigen in patients' whole-blood specimens as an alternative diagnostic approach. J Infect Dis 2017; 216: 182-190.
30 Lum F-M, Lye D C B, Tan J J L et al. Longitudinal study of cellular and systemic cytokine signatures to define the dynamics of a balanced immune environment during disease manifestation in Zika virus-infected patients. J Infect Dis 2018; 218: 814-824.
31 Vaughan K, Greenbaum J, Blythe M, Peters B, Sette A. Meta-analysis of all immune epitope data in the Flavivirus genus: inventory of current immune epitope data status in the context of virus immunity and immunopathology. Viral Immunol 2010; 23: 259-284.
32 Low J G H, Ooi E-E, Tolfvenstam T et al. Early dengue infection and outcome study (EDEN)—study design and preliminary findings. Ann Acad Med Singapore 2006; 35: 783-789.
33 Koraka P, Suharti C, Setiati T E et al. Kinetics of dengue virus-specific serum immunoglobulin classes and subclasses correlate with clinical outcome of infection. J Clin Microbiol 2001; 39: 4332-4338.
34 Montoya M, Collins M, Dejnirattisai W et al. Longitudinal analysis of antibody cross-neutralization following Zika virus and dengue virus infection in Asia and the Americas. J Infect Dis 2018; 218: 536-545.
35 Martin-Acebes M A, Saiz J-C, Jiménez de Oya N. Antibody-dependent enhancement and Zika: real threat or phantom menace? Front Cell Infect Microbiol 2018; 8: 2014-2017.
36 Terzian A C B, Schanoski A S, Mota M T O et al. Viral load and cytokine response profile does not support antibody-dependent enhancement in dengue-primed Zika virus-infected patients. Clin Infect Dis 2017; 65: 1260-1265.
37 Kuhn R J, Dowd K A, Beth C, Pierson T C. Shake, rattle, and roll: impact of the dynamics of flavivirus particles on their interactions with the host. Virology 2015; 480: 508-517.
38 Ramanathan B, Poh C L, Kirk K, McBride W J H, Aaskov J, Grollo L. Synthetic B-cell epitopes eliciting cross-neutralizing antibodies: strategies for future dengue vaccine. PLoS One 2016; 11: 1-22.
39 Crill W D, Chang G J. Localization and characterization of Flavivirus envelope glycoprotein. Cell 2004; 78: 13975-13986.
40 Sun E C, Zhao J, Liu N H et al. Comprehensive mapping of west nile virus (WNV)- and japanese encephalitis virus serocomplex-specific linear B-cell epitopes from WNV non-structural protein 1. J Gen Virol 2012; 93: 50-60.
41 Lee H-J, Cho Y, Kang H J et al. Identification of peptide based B-cell epitopes in Zika virus NS1. Biochem Biophys Res Commun 2018; 505: 1010-1014.
42 Freire M C L C, Pol-Fachin L, Coêlho D F et al. Mapping putative B-cell Zika virus NS1 epitopes provides molecular basis for anti-NS1 antibody discrimination between Zika and dengue viruses. ACS Omega 2017; 2: 3913-3920.
43 Song H, Qi J, Haywood J, Shi Y, Gao G F. Zika virus NS1 structure reveals diversity of electrostatic surfaces among flaviviruses. Nat Struct Mol Biol 2016; 23: 456-458.
44 Bujang M A, Adnan T H. Requirements for minimum sample size for sensitivity and specificity analysis. J Clin Diagnostic Res 2016; 10: 1-6.
45 Wu H C, Huang Y L, Chao T T et al. Identification of B-cell epitope of dengue virus type 1 and its application in diagnosis of patients. J Clin Microbiol 2001; 39: 977-982.
46 Magalhaes T, Yen C-W, Marin K et al. Rapid antigen tests for dengue virus serotypes and Zika virus in patient serum. Sci Transl Med 2017; 9: eaan1589.
47 Her Z, Kam Y W, Gan V C et al. Severity of plasma leakage is associated with high levels of interferon γ-inducible protein 10, hepatocyte growth factor, matrix metalloproteinase 2 (MMP-2), and MMP-9 during dengue virus infection. J Infect Dis 2017; 215: 42-51.
48 Messina J P, Brady O J, Scott T W et al. Global spread of dengue virus types: mapping the 70 year history. Trends Microbiol 2014; 22: 138-146.
49 Suwandono A, Kosasih H, Nurhayati et al. Four dengue virus serotypes found circulating during an outbreak of dengue fever and dengue haemorrhagic fever in Jakarta, Indonesia, during 2004. Trans R Soc Trop Med Hyg 2006; 100: 855-862.
50 Ng L-C, Chem Y-K, Koo C et al. 2013 dengue outbreaks in Singapore and Malaysia caused by different viral strains. Am J Trop Med Hyg 2015; 92: 1150-1155.
51 Rajarethinam J, Ang L, Ong J et al. Dengue in Singapore from 2004 to 2016: cyclical epidemic patterns dominated by serotypes 1 and 2. Am J Trop Med Hyg 2018; 99: 204-210.
52 Her Z, Malleret B, Chan M et al. Active infection of human blood monocytes by chikungunya virus triggers an innate immune response. J Immunol 2010; 184: 5903-5913.
53 Hamel R, Dejamac O, Wichit S et al. Biology of Zika virus infection in human skin cells. J Virol 2015; 89: 8880-8896.
54 Ang L W, Kam Y W, Lin C et al. Seroprevalence of antibodies against chikungunya virus in Singapore resident adult population. PLoS Negl Trop Dis 2017; 11: 1-13.
55 Kelley L A, Mezulis S, Yates C M, Wass M N, Stenberg M J E. The Phyre2 web portal for protein modeling, prediction and analysis. Nat Protoc 2015; 10: 845.

TABLE 7
Sequence of peptide pairs

| | | Amino | | | | | |
|---|---|---|---|---|---|---|---|
| | | acid | | | | | |
| | | position | | | | Peptide | |
| | | on ZIKV | | | | similarity % | Classi- |
| | | (accession | | Corresponding | Corresponding | identity, % | fication |
| | Peptide | KJ776791) | | ZIKV | DENV | query | of potential |
| Protein | no | Start | End | sequence | sequence | cover) | epitope |
| prM | 7 | 56 | 72 | LDEGVEPDDVDCWCNTT | HITEVEPEDIDCWCNLT | 82%, | Common |
| | | | | | | 64% | flavivirus |
| NS1 | 36 | 70 | 85 | SVEGELNAILEENGVQ | QIANELNYILWENNIK | 70%, | Common |
| | | | | | | 56% | flavivirus |
| NS1 | 38 | 119 | 136 | GKSYFVRAAKTNNSFVVD | GKAKIVTAETQNSSFIID | 57%, | Common |
| | | | | | | 38% | flavivirus |
| NS1 | 39 | 137 | 154 | GDTLKECPLKHRAWNSFL | GPNTPECPSASRAWNVWE | 70%, | Common |
| | | | | | | 55% | flavivirus |
| NS1 | 46 | 284 | 301 | KVHVEETCGTRGPSLRST | TVVITENCGTRGPSLRTT | 85%, | Common |
| | | | | | | 72% | flavivirus |
| NS1 | 49 | 315 | 326 | ECTMPPLSFRAK | SCTLPPLRYMGE | 83%, | Common |
| | | | | | | 50% | flavivirus |
| prM | 3 | 24 | 41 | SFPTTLGMNKCYIQIMDL | LFKTASGINMCTLIAMDL | No | ZIKV- |
| | | | | | | similarity | specific |
| E | 26 | 271 | 288 | GALEAEMDGAKGRLSSGH | GATEIQNSGGTSIFAGH | 100%, | ZIKV- |
| | | | | | | 11% | specific |
| E | 32 | 453 | 470 | FKSLFGGMSWFSQILIGT | YTALFSGVSwVMKIGIGV | 71%, | ZIKV- |
| | | | | | | 38% | specific |
| prM | 9 | 78 | 92 | YGTCHHKKGEARRSR | TSTwVTYGTCNQAG | 67%, | DENV- |
| | | | | | | 40% | specific |
| E | 17 | 131 | 149 | PENLEYRIMLSVHGSQHS | YENLKYTVIITVHTGDQH | 53%, | DENV- |
| | | | | | | 88% | specific |
| NS1 | 43 | 257 | 274 | REGYRTQMKGPWHSEELE | RPGYHTQTAGPWHLGKLE | 69%, | DENV- |
| | | | | | | 72% | specific |
| NS1 | 45 | 275 | 292 | IRFEECPGTKVHVEETCG | LDFNYCEGTTVVITENCG | 54%, | DENV- |
| | | | | | | 72% | specific |

TABLE 8
Sequences and their corresponding sequence identity numbers (SEQ ID NOs.)

| | | Corresponding | SEQ | Corresponding | SEQ |
|---|---|---|---|---|---|
| | Peptide | ZIKV | ID | DENV | ID |
| Protein | no | sequence | NO. | sequence | NO. |
| prM | 1 | RGSAYYMYLDRNDAGEAI | 1 | RDGEPRMIVGKNERGKSL | 52 |
| | 2 | DRNDAGEAISFPTTLGMN | 2 | GKNERGKSLLFKTASGIN | 53 |
| | 3 | SFPTTLGMNKCYIQIMDL | 3 | LFKTASGINMCTLIAMDL | 54 |
| | 4 | KCYIQIMDLGHMCDATMS | 4 | MCTLIAMDLGEMCDDTVT | 55 |
| | 5 | GHMCDATMSYECPMLDEG | 5 | GEMCDDTVTYKCPHITE | 56 |
| | 6 | YECPMLDEGVEPDDVDCW | 6 | YKCPHITEVEPEDIDCW | 57 |
| | 7 | LDEGVEPDDVDCWCNTT | 7 | HITEVEPEDIDCWCNLT | 58 |
| | 8 | CNTTSTWVVYGTCHHKKG | 8 | CNLTSTWVTYGTCNQAG | 59 |
| | 9 | YGTCHHKKGEARRSR | 9 | TSTWVTYGTCNQAG | 60 |
| | 10 | HSTRKLQTRSQTWLESREY | 10 | VGMGLDTRTQTWMSAEGAW | 61 |
| E | 11 | DKPTVDIELVTTTVSNMA | 11 | NKPTLDIELQKTEATQLA | 62 |
| | 12 | YEASISDMASDS | 12 | IEGKITNITTDS | 63 |
| | 13 | RCPTQGEAYLDKQSDTQY | 13 | RCPTQGEAVLPEEQDQNY | 64 |
| | 14 | VCKRTLVDRGWGNGCGLF | 14 | VCKHTYVDRGWGNGCGLF | 65 |
| | 15 | LVTCAKFACSKKMTGKSI | 15 | LVTCAKFQCLEPIEGKVV | 66 |
| | 16 | KKMTGKSIQPENLEYRIM | 16 | EPIEGKVVQYENLKYTVI | 67 |
| | 17 | PENLEYRIMLSVHGSQHS | 17 | YENLKYTVIITVHTGDQH | 68 |
| | 18 | SGMIVNDTGHETDENRAK | 18 | GDQHQVGNETQGVTAEIT | 69 |
| | 19 | GHETDENRAKVEITPNSP | 19 | GNETQGVTAEITPQASTT | 70 |
| | 20 | KVEITPNSPRAEATLGGF | 20 | TAEITPQASTTEAILPEY | 71 |
| | 21 | EPRTGLDFSDLYY | 21 | SPRTGLDFNEMIL | 72 |
| | 22 | SDLYYLTMNNKHWLVHKE | 22 | NEMILLTMKNKAWMVHRQ | 73 |
| | 23 | WFHDIPLPWHAGADTGTP | 23 | WFFDLPLPWTSGATTETP | 74 |
| | 24 | HWNNKEALVEF | 24 | TWNRKELLVTF | 75 |
| | 25 | EFKDAHAKRQTVVVLGSQ | 25 | TFKNAHAKKQEVVVLGSQ | 76 |
| | 26 | GALEAEMDGAKGRLSSGH | 26 | GATEIQNSGGTSIFAGH | 77 |
| | 27 | SLCTAAFTFTKIPA | 27 | AMCTNTFVLKKEVS | 78 |
| | 28 | TVTVEVQYAGTDGPCKVP | 28 | TILIKVEYKGEDAPCKIP | 79 |
| | 29 | AQMAVDMQTLTPV | 29 | FSTEDGQGKAHN | 80 |
| | 30 | ANPVITESTENSKMMLEL | 30 | ANPVVTKKEEPVNIEA | 81 |
| | 31 | RSGSTIGKAFEATVRGAK | 31 | KKGSSIGKMFEATARGAR | 82 |
| | 32 | FKSLFGGMSWFSQILIGT | 32 | YTALFSGVSWVMKIGIGV | 83 |
| NS1 | 33 | DVGCSVDFSKKETRCGTG | 33 | DMGCVINWKGKELKCGSG | 84 |
| | 34 | VFVYNDVEAWRDRYKYHP | 34 | IFVTNEVHTVVTEQYKFQA | 85 |
| | 35 | CGISSVSRMENIMWRSVE | 35 | CGIRSTTRMENLLWKQIA | 86 |
| | 36 | SVEGELNAILEENGVQ | 36 | QIANELNYILWENNIK | 87 |
| | 37 | GSVKNPMWRGPQRLPVPVNELP | 37 | GDIIGVLEQGKRTLTPQPMELK | 88 |
| | 38 | GKSYFVRAAKTNNSFVVD | 38 | GKAKIVTAETQNSSFIID | 89 |
| | 39 | GDTLKECPLKHRAWNSFL | 39 | GPNTPECPSASRAWNVWE | 90 |
| | 40 | VEDHGFGVFHTSVWLKVREDYS | 40 | VEDYGFGVFTTNIWLKLREVYT | 91 |
| | 41 | SDLIIPKSLAGPLSHHNT | 41 | SDMIIPKSLAGPISQHNH | 92 |
| | 42 | AGPLSHHNTREGYRTQMK | 42 | AGPISQHNHRPGYHTQTA | 93 |
| | 43 | REGYRTQMKGPWHSEELE | 43 | RPGYHTQTAGPWHLGKLE | 94 |
| | 44 | SEELEIRFEECP | 44 | LGKLELDFNYCE | 95 |
| | 45 | IRFEECPGTKVHVEETCG | 45 | LDFNYCEGTTVVITENCG | 96 |
| | 46 | KVHVEETCGTRGPSLRST | 46 | TVVITENCGTRGPSLRTT | 97 |
| | 47 | TRGPSLRSTTASGRVIEE | 47 | TRGPSLRTTTVSGKLIHE | 98 |
| | 48 | TASGRVIEEWCCRECTMP | 48 | TVSGKLIHEWCCRSCTLP | 99 |
| | 49 | ECTMPPLSFRAK | 49 | SCTLPPLRYMGE | 100 |
| | 50 | PLSFRAKDGCVVYGMEIRP | 50 | PLRYMGEDGCVVYGMEIRP | 101 |
| | 51 | RKEPESNLVRSMVTAG | 51 | ISEKEENMVKSLVSAG | 102 |

### APPLICATIONS

In the present disclosure, antibody and neutralising responses by ZIKV patients from Singapore were characterised longitudinally. Common and differential linear B-cell epitopes recognised by antibodies from Singapore ZIKV and DENV patients were then identified. Importantly, the potential value of these identified epitopes in a diagnostic setting was further assessed using sera from patients from Thailand previously diagnosed with DENV, bacterial, and including those of unknown infections.

This present disclosure furthers the development of a serology-driven differential flavivirus diagnosis, particularly between ZIKV and DENV, allowing for accurate diagnosis that will improve patient management. The application can also be further expanded to study sero-prevalence and vaccine strategies.

Embodiments of the method advantageously provide a proper serology diagnostic tool that is able to accurately identify a flavivirus infection such as ZIKV and/or DENV, or differentiate between the two flavivirus infections such as between ZIKV and DENV.

1. A method of identifying a flavivirus infection selected from Zika virus ZIKV), dengue virus (DENV) and combination thereof in a subject, the method comprising:
determining whether a sample of the subject reacts with a peptide P that is capable of giving a relative binding capacity BCᵣₑₗₐₜᵢᵥₑ≤0.05 or ≥0.1, wherein
$${BC}_{relative} = \frac{{R}_{P 1 z} - {R}_{P 2 z}}{{R}_{P 2 z}} - \frac{{R}_{P 1 d} - {R}_{P 2 d}}{{R}_{P 2 d}}$$
(i) wherein where the peptide P comprises a ZIKV-derived peptide,
R_{P1z}=response of a ZIKV-induced antigen binding protein with peptide P,
R_{P2z}=response of the ZIKV-induced antigen binding protein with a corresponding peptide C comprising a DENV-derived peptide having a sequence homologous to the peptide P,
R_{P1d}=response of a DENV-induced antigen binding protein with the peptide P,
R_{P2d}=response of the DENV-induced antigen binding protein with the corresponding peptide C comprising a DENV-derived peptide having a sequence homologous to the peptide P,
$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein ,$$
or
(ii) wherein where the peptide P comprises a DENV-derived peptide,
R_{P1z}=response of a ZIKV-induced antigen binding protein with a corresponding peptide C comprising a ZIKV-derived peptide having a sequence homologous to the peptide P,
R_{P2z}=response of the ZIKV-induced antigen binding protein with the peptide P,
R_{P1d}=response of a DENV-induced antigen binding protein with the corresponding peptide C comprising a ZIKV-derived peptide having a sequence homologous to the peptide P,
R_{P2d}=response of the DENV-induced antigen binding protein with the peptide P,
$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein .$$

2. The method of claim 1, wherein the peptide P comprises an epitope on a prM protein, an E glycoprotein or a NS1 protein of ZIKV or DENV, optionally wherein the epitope is located in a solvent-exposed region of the prM protein, the E glycoprotein or the NS1 protein.

3. (canceled)

4. The method of claim 1, wherein the peptide P is from 5 to 25 amino acids long.

5. The method of claim 1, wherein the peptide P shares no more than 50% sequence similarity with the corresponding peptide C.

6. The method of claim 1, wherein the peptide P comprises
one or more ZIKV-derived peptide selected from the group consisting of:
SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);
a peptide sharing at least 75% sequence identity thereto; or
a peptide differing by one, two, three or four amino acids thereto; or
portions thereof; and/or
one or more DENV-derived peptide selected from the group consisting of:
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);
or
a peptide sharing at least 75% sequence identity thereto; or
a peptide differing by one, two, three or four amino acids thereto; or
portions thereof.

7. The method of claim 1, wherein the method comprises determining whether the sample reacts with one or more peptide selected from the group consisting of:
SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
a peptide sharing at least 75% sequence identity thereto; or
a peptide differing by one, two, three or four amino acids thereto; or
portions thereof; and/or
wherein where the sample reacts with the one or more peptide or the portions thereof, the subject is indicated for flavivirus infection.

8. The method of claim 1, wherein the method is a method of identifying a flavivirus infection in an acute phase, and the method comprises determining whether the sample reacts with one or more peptide selected from the group consisting of:
SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
wherein where the sample reacts with the one or more peptide or the portions thereof, the subject is indicated for flavivirus infection in an acute phase.

9. The method of claim 1, wherein the method comprises determining whether the sample reacts with one or more peptide selected from the group consisting of:
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
a peptide sharing at least 75% sequence identity thereto; or
a peptide differing by one, two, three or four amino acids thereto; or
portions thereof;
wherein where the sample reacts with the one or more peptide or the portions thereof, the subject is indicated for ZIKV infection.

10. The method of claim 1, wherein the method is a method of identifying a ZIKV infection in an acute phase, and the method comprises determining whether the sample reacts with:
SEQ ID NO: 32
(FKSLFGGMSWFSQILIGT);
a peptide sharing at least 75% sequence identity thereto; or
a peptide differing by one, two, three or four amino acids thereto; or
portions thereof;
wherein where the sample reacts with the peptide or the portions thereof, the subject is indicated for ZIKV infection in an acute phase.

11. The method of claim 1, wherein the method comprises determining whether the sample reacts with one or more peptide selected from the group consisting of:
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);
and/or
a peptide sharing at least 75% sequence identity thereto; or
a peptide differing by one, two, three or four amino acids thereto; or
portions thereof;
wherein where the sample reacts with the one or more peptide or the portions thereof, the subject is indicated for DENV infection.

12. The method of claim 1, wherein determining whether the sample reacts with the peptide P comprises performing an immunoassay to assess whether antigen-binding proteins that are capable of binding to the peptide are present in the sample.

13. The method of claim 1, the method further comprising administering to the subject a ZIKV and/or DENV treatment regimen if the subject is indicated for ZIKV and/or DENV infection.

14. A kit for identifying a flavivirus infection selected from Zika virus, dengue virus and combination thereof in a subject, the kit comprising a peptide P that is capable of giving a relative binding capacity BCᵣₑₗₐₜᵢᵥₑ≤0.05 or ≥0.1, wherein
$${BC}_{relative} = \frac{{R}_{P 1 z} - {R}_{P 2 z}}{{R}_{P 2 z}} - \frac{{R}_{P 1 d} - {R}_{P 2 d}}{{R}_{P 2 d}}$$
wherein where the peptide P comprises a ZIKV-derived peptide,
R_{P1z}=response of a ZIKV-induced antigen binding protein with peptide P,
R_{P2z}=response of the ZIKV-induced antigen binding protein with a corresponding peptide C comprising a DENV-derived peptide having a sequence homologous to the peptide P,
R_{P1d}=response of a DENV-induced antigen binding protein with the peptide P,
R_{P2d}=response of the DENV-induced antigen binding protein with the corresponding peptide C comprising a DENV-derived peptide having a sequence homologous to the peptide P,
$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein ,$$
wherein where the peptide P comprises a DENV-derived peptide,
R_{P1z}=response of a ZIKV-induced antigen binding protein with a corresponding peptide C comprising a ZIKV-derived peptide having a sequence homologous to the peptide P,
R_{P2z}=response of the ZIKV-induced antigen binding protein with the peptide P,
R_{P1d}=response of a DENV-induced antigen binding protein with the corresponding peptide C comprising a ZIKV-derived peptide having a sequence homologous to the peptide P,
R_{P2d}=response of the DENV-induced antigen binding protein with the peptide P,
$$\frac{{R}_{P 1 z} - {R}_{P 2 z}}{{R}_{P 2 z}} = binding capacity of the \text{ZIKV-induced} antigen binding protein , \\ \frac{{R}_{P 1 d} - {R}_{P 2 d}}{{R}_{P 2 d}} = binding capacity of the \text{DENV-induced} antigen binding protein .$$

15. The kit of claim 14, wherein the peptide P comprises
one or more ZIKV-derived peptide selected from the group consisting of:
SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);
a peptide sharing at least 75% sequence identity thereto; or
a peptide differing by one, two, three or four amino acids thereto; or
portions thereof; and/or
one or more DENV-derived peptide selected from the group consisting of:
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);
or
a peptide sharing at least 75% sequence identity thereto; or
a peptide differing by one, two, three or four amino acids thereto; or
portions thereof.

16. The kit of claim 14 further comprising one or more of the following:
a plate coated with a capture agent for anti-ZIKV and/or anti-DENV, and
a detection agent for detecting the presence of captured anti-ZIKV and/or anti-DENV,
wherein the capture agent and the detection agent comprise a ZIKV and/or DENV antigen and/or an anti-immunoglobulin.

17. The method of claim 1, wherein the subject comprises an Asian subject.

18. A composition comprising an isolated peptide or an immune system stimulating composition comprising an isolated peptide, wherein said isolated peptide is selected from the group consisting of:
SEQ ID NO: 7
(LDEGVEPDDVDCWCNTT);
SEQ ID NO: 36
(SVEGELNAILEENGVQ);
SEQ ID NO: 38
(GKSYFVRAAKTNNSFVVD);
SEQ ID NO: 39
(GDTLKECPLKHRAWNSFL);
SEQ ID NO: 46
(KVHVEETCGTRGPSLRST);
SEQ ID NO: 49
(ECTMPPLSFRAK);
SEQ ID NO: 3
(SFPTTLGMNKCYIQIMDL);
SEQ ID NO: 26
(GALEAEMDGAKGRLSSGH);
SEQ ID NO: 32
(FKSLFGGMSWFSQILIGT);
SEQ ID NO: 9
(YGTCHHKKGEARRSR);
SEQ ID NO: 17
(PENLEYRIMLSVHGSQHS);
SEQ ID NO: 43
(REGYRTQMKGPWHSEELE);
SEQ ID NO: 45
(IRFEECPGTKVHVEETCG);
SEQ ID NO: 58
(HITEVEPEDIDCWCNLT);
SEQ ID NO: 87
(QIANELNYILWENNIK);
SEQ ID NO: 89
(GKAKIVTAETQNSSFIID);
SEQ ID NO: 90
(GPNTPECPSASRAWNVWE);
SEQ ID NO: 97
(TVVITENCGTRGPSLRTT);
SEQ ID NO: 100
(SCTLPPLRYMGE);
SEQ ID NO: 54
(LFKTASGINMCTLIAMDL);
SEQ ID NO: 77
(GATEIQNSGGTSIFAGH);
SEQ ID NO: 83
(YTALFSGVSWVMKIGIGV);
SEQ ID NO: 60
(TSTWVTYGTCNQAG);
SEQ ID NO: 68
(YENLKYTVIITVHTGDQH);
SEQ ID NO: 94
(RPGYHTQTAGPWHLGKLE);
SEQ ID NO: 96
(LDFNYCEGTTVVITENCG);
or
a peptide sharing at least 75% sequence identity thereto; or
a peptide differing by one, two, three or four amino acids thereto; or
portions thereof.

19. (canceled)

20. The method of claim 1, wherein the method further distinguishes ZIKV infection from DENV infection in a subject, the method comprising:
determining whether a sample of the subject reacts with a peptide of SEQ ID NO: 32 (FKSLFGGMSWFSQILIGT); a peptide sharing at least 75% sequence identity thereto; or a peptide differing by one, two, three or four amino acids thereto; or portions thereof, wherein where the sample reacts with the peptide or the portions thereof, the subject is indicated for ZIKV infection; and/or
determining whether a sample of the subject reacts with a peptide of SEQ ID NO: 9 (YGTCHHKKGEARRSR); a peptide sharing at least 75% sequence identity thereto; or a peptide differing by one, two, three or four amino acids thereto; or portions thereof, wherein where the sample reacts with the peptide or the portions thereof, the subject is indicated for DENV infection.

21. The kit of claim 14, wherein the subject comprises an Asian subject.


SEQUENCE LISTING (102 sequences)

SEQ ID NO: 1 - protein, 18 aa, Flavivirus
RGSAYYMYLDRNDAGEAI

SEQ ID NO: 2 - protein, 18 aa, Flavivirus
DRNDAGEAISFPTTLGMN

SEQ ID NO: 3 - protein, 18 aa, Flavivirus
SFPTTLGMNKCYIQIMDL

SEQ ID NO: 4 - protein, 18 aa, Flavivirus
KCYIQIMDLGHMCDATMS

SEQ ID NO: 5 - protein, 18 aa, Flavivirus
GHMCDATMSYECPMLDEG

SEQ ID NO: 6 - protein, 18 aa, Flavivirus
YECPMLDEGVEPDDVDCW

SEQ ID NO: 7 - protein, 17 aa, Flavivirus
LDEGVEPDDVDCWCNTT

SEQ ID NO: 8 - protein, 18 aa, Flavivirus
CNTTSTWVVYGTCHHKKG

SEQ ID NO: 9 - protein, 15 aa, Flavivirus
YGTCHHKKGEARRSR

SEQ ID NO: 10 - protein, 19 aa, Flavivirus
HSTRKLQTRSQTWLESREY

SEQ ID NO: 11 - protein, 18 aa, Flavivirus
DKPTVDIELVTTTVSNMA

SEQ ID NO: 12 - protein, 12 aa, Flavivirus
YEASISDMASDS

SEQ ID NO: 13 - protein, 18 aa, Flavivirus
RCPTQGEAYLDKQSDTQY

SEQ ID NO: 14 - protein, 18 aa, Flavivirus
VCKRTLVDRGWGNGCGLF

SEQ ID NO: 15 - protein, 18 aa, Flavivirus
LVTCAKFACSKKMTGKSI

SEQ ID NO: 16 - protein, 18 aa, Flavivirus
KKMTGKSIQPENLEYRIM

SEQ ID NO: 17 - protein, 18 aa, Flavivirus
PENLEYRIMLSVHGSQHS

SEQ ID NO: 18 - protein, 18 aa, Flavivirus
SGMIVNDTGHETDENRAK

SEQ ID NO: 19 - protein, 18 aa, Flavivirus
GHETDENRAKVEITPNSP

SEQ ID NO: 20 - protein, 18 aa, Flavivirus
KVEITPNSPRAEATLGGF

SEQ ID NO: 21 - protein, 13 aa, Flavivirus
EPRTGLDFSDLYY

SEQ ID NO: 22 - protein, 18 aa, Flavivirus
SDLYYLTMNNKHWLVHKE

SEQ ID NO: 23 - protein, 18 aa, Flavivirus
WFHDIPLPWHAGADTGTP

SEQ ID NO: 24 - protein, 11 aa, Flavivirus
HWNNKEALVEF

SEQ ID NO: 25 - protein, 18 aa, Flavivirus
EFKDAHAKRQTVVVLGSQ

SEQ ID NO: 26 - protein, 18 aa, Flavivirus
GALEAEMDGAKGRLSSGH

SEQ ID NO: 27 - protein, 14 aa, Flavivirus
SLCTAAFTFTKIPA

SEQ ID NO: 28 - protein, 18 aa, Flavivirus
TVTVEVQYAGTDGPCKVP

SEQ ID NO: 29 - protein, 13 aa, Flavivirus
AQMAVDMQTLTPV

SEQ ID NO: 30 - protein, 18 aa, Flavivirus
ANPVITESTENSKMMLEL

SEQ ID NO: 31 - protein, 18 aa, Flavivirus
RSGSTIGKAFEATVRGAK

SEQ ID NO: 32 - protein, 18 aa, Flavivirus
FKSLFGGMSWFSQILIGT

SEQ ID NO: 33 - protein, 18 aa, Flavivirus
DVGCSVDFSKKETRCGTG

SEQ ID NO: 34 - protein, 18 aa, Flavivirus
VFVYNDVEAWRDRYKYHP

SEQ ID NO: 35 - protein, 18 aa, Flavivirus
CGISSVSRMENIMWRSVE

SEQ ID NO: 36 - protein, 16 aa, Flavivirus
SVEGELNAILEENGVQ

SEQ ID NO: 37 - protein, 22 aa, Flavivirus
GSVKNPMWRGPQRLPVPVNELP

SEQ ID NO: 38 - protein, 18 aa, Flavivirus
GKSYFVRAAKTNNSFVVD

SEQ ID NO: 39 - protein, 18 aa, Flavivirus
GDTLKECPLKHRAWNSFL

SEQ ID NO: 40 - protein, 22 aa, Flavivirus
VEDHGFGVFHTSVWLKVREDYS

SEQ ID NO: 41 - protein, 18 aa, Flavivirus
SDLIIPKSLAGPLSHHNT

SEQ ID NO: 42 - protein, 18 aa, Flavivirus
AGPLSHHNTREGYRTQMK

SEQ ID NO: 43 - protein, 18 aa, Flavivirus
REGYRTQMKGPWHSEELE

SEQ ID NO: 44 - protein, 12 aa, Flavivirus
SEELEIRFEECP

SEQ ID NO: 45 - protein, 18 aa, Flavivirus
IRFEECPGTKVHVEETCG

SEQ ID NO: 46 - protein, 18 aa, Flavivirus
KVHVEETCGTRGPSLRST

SEQ ID NO: 47 - protein, 18 aa, Flavivirus
TRGPSLRSTTASGRVIEE

SEQ ID NO: 48 - protein, 18 aa, Flavivirus
TASGRVIEEWCCRECTMP

SEQ ID NO: 49 - protein, 12 aa, Flavivirus
ECTMPPLSFRAK

SEQ ID NO: 50 - protein, 18 aa, Flavivirus
PLSFRAKDGCWYGMEIRP

SEQ ID NO: 51 - protein, 16 aa, Flavivirus
RKEPESNLVRSMVTAG

SEQ ID NO: 52 - protein, 18 aa, Flavivirus
RDGEPRMIVGKNERGKSL

SEQ ID NO: 53 - protein, 18 aa, Flavivirus
GKNERGKSLLFKTASGIN

SEQ ID NO: 54 - protein, 18 aa, Flavivirus
LFKTASGINMCTLIAMDL

SEQ ID NO: 55 - protein, 18 aa, Flavivirus
MCTLIAMDLGEMCDDTVT

SEQ ID NO: 56 - protein, 17 aa, Flavivirus
GEMCDDTVTYKCPHITE

SEQ ID NO: 57 - protein, 17 aa, Flavivirus
YKCPHITEVEPEDIDCW

SEQ ID NO: 58 - protein, 17 aa, Flavivirus
HITEVEPEDIDCWCNLT

SEQ ID NO: 59 - protein, 17 aa, Flavivirus
CNLTSTWVTYGTCNQAG

SEQ ID NO: 60 - protein, 14 aa, Flavivirus
TSTWVTYGTCNQAG

SEQ ID NO: 61 - protein, 19 aa, Flavivirus
VGMGLDTRTQTWMSAEGAW

SEQ ID NO: 62 - protein, 18 aa, Flavivirus
NKPTLDIELQKTEATQLA

SEQ ID NO: 63 - protein, 12 aa, Flavivirus
IEGKITNITTDS

SEQ ID NO: 64 - protein, 18 aa, Flavivirus
RCPTQGEAVLPEEQDQNY

SEQ ID NO: 65 - protein, 18 aa, Flavivirus
VCKHTYVDRGWGNGCGLF

SEQ ID NO: 66 - protein, 18 aa, Flavivirus
LVTCAKFQCLEPIEGKVV

SEQ ID NO: 67 - protein, 18 aa, Flavivirus
EPIEGKVVQYENLKYTVI

SEQ ID NO: 68 - protein, 18 aa, Flavivirus
YENLKYTVIITVHTGDQH

SEQ ID NO: 69 - protein, 18 aa, Flavivirus
GDQHQVGNETQGVTAEIT

SEQ ID NO: 70 - protein, 18 aa, Flavivirus
GNETQGVTAEITPQASTT

SEQ ID NO: 71 - protein, 18 aa, Flavivirus
TAEITPQASTTEAILPEY

SEQ ID NO: 72 - protein, 13 aa, Flavivirus
SPRTGLDFNEMIL

SEQ ID NO: 73 - protein, 18 aa, Flavivirus
NEMILLTMKNKAWMVHRQ

SEQ ID NO: 74 - protein, 18 aa, Flavivirus
WFFDLPLPWTSGATTETP

SEQ ID NO: 75 - protein, 11 aa, Flavivirus
TWNRKELLVTF

SEQ ID NO: 76 - protein, 18 aa, Flavivirus
TFKNAHAKKQEVVVLGSQ

SEQ ID NO: 77 - protein, 17 aa, Flavivirus
GATEIQNSGGTSIFAGH

SEQ ID NO: 78 - protein, 14 aa, Flavivirus
AMCTNTFVLKKEVS

SEQ ID NO: 79 - protein, 18 aa, Flavivirus
TILIKVEYKGEDAPCKIP

SEQ ID NO: 80 - protein, 12 aa, Flavivirus
FSTEDGQGKAHN

SEQ ID NO: 81 - protein, 16 aa, Flavivirus
ANPVVTKKEEPVNIEA

SEQ ID NO: 82 - protein, 18 aa, Flavivirus
KKGSSIGKMFEATARGAR

SEQ ID NO: 83 - protein, 18 aa, Flavivirus
YTALFSGVSWVMKIGIGV

SEQ ID NO: 84 - protein, 18 aa, Flavivirus
DMGCVINWKGKELKCGSG

SEQ ID NO: 85 - protein, 18 aa, Flavivirus
IFVTNEVHTWTEQYKFQA

SEQ ID NO: 86 - protein, 18 aa, Flavivirus
CGIRSTTRMENLLWKQIA

SEQ ID NO: 87 - protein, 16 aa, Flavivirus
QIANELNYILWENNIK

SEQ ID NO: 88 - protein, 22 aa, Flavivirus
GDIIGVLEQGKRTLTPQPMELK

SEQ ID NO: 89 - protein, 18 aa, Flavivirus
GKAKIVTAETQNSSFIID

SEQ ID NO: 90 - protein, 18 aa, Flavivirus
GPNTPECPSASRAWNVWE

SEQ ID NO: 91 - protein, 22 aa, Flavivirus
VEDYGFGVFTTNIWLKLREVYT

SEQ ID NO: 92 - protein, 18 aa, Flavivirus
SDMIIPKSLAGPISQHNH

SEQ ID NO: 93 - protein, 18 aa, Flavivirus
AGPISQHNHRPGYHTQTA

SEQ ID NO: 94 - protein, 18 aa, Flavivirus
RPGYHTQTAGPWHLGKLE

SEQ ID NO: 95 - protein, 12 aa, Flavivirus
LGKLELDFNYCE

SEQ ID NO: 96 - protein, 18 aa, Flavivirus
LDFNYCEGTTVVITENCG

SEQ ID NO: 97 - protein, 18 aa, Flavivirus
TVVITENCGTRGPSLRTT

SEQ ID NO: 98 - protein, 18 aa, Flavivirus
TRGPSLRTTTVSGKLIHE

SEQ ID NO: 99 - protein, 18 aa, Flavivirus
TVSGKLIHEWCCRSCTLP

SEQ ID NO: 100 - protein, 12 aa, Flavivirus
SCTLPPLRYMGE

SEQ ID NO: 101 - protein, 18 aa, Flavivirus
PLRYMGEDGCWYGMEIRP

SEQ ID NO: 102 - protein, 16 aa, Flavivirus
ISEKEENMVKSLVSAG